(19) United States
(12) Patent Application Publication
(10) Pub. No.: US 2024/0115699 A1
(43) Pub. Date: Apr. 11, 2024

(54) USE OF CANCER CELL EXPRESSION OF CADHERIN 12 AND CADHERIN 18 TO TREAT MUSCLE INVASIVE AND METASTATIC BLADDER CANCERS

(71) Applicant: Cedars-Sinai Medical Center, Los Angeles, CA (US)

(72) Inventors: Dan Theodorescu, Los Angeles, CA (US); Simon Knott, Topanga, CA (US); Kenneth Gouin, Los Angeles, CA (US); Nathan Ing, Los Angeles, CA (US); Charles Rosser, Stevenson Ranch, CA (US)

(73) Assignee: Cedars-Sinai Medical Center, Los Angeles, CA (US)

(21) Appl. No.: 18/289,534

(22) PCT Filed: Jun. 6, 2022
(86) PCT No.: PCT/US2022/032382

§ 371 Date: Nov. 3, 2023

Related U.S. Application Data
(60) Provisional application No. 63/197,129, filed on Jun. 4, 2021.

(51) Int. Cl. A61K 39/395; A61K 31/337; A61K 31/475; A61K 31/519; A61K 31/704; A61K 31/7068; A61K 33/243; A61P 35/00; C12Q 1/6874; C12Q 1/6886; G01N 33/574

(52) U.S. Cl. CPC A61K 39/3955; A61K 31/337; A61K 31/475; A61K 31/519; A61K 31/704; A61K 31/7068; A61K 33/243; A61P 35/00; C12Q 1/6874; C12Q 1/6886; G01N 33/57492; C12Q 2600/106; C12Q 2600/112; C12Q 2600/158

(57) ABSTRACT

We combined single nuclei RNA sequencing with spatial transcriptomics and single-cell resolution spatial proteomic analysis of human bladder cancer to identify an epithelial subpopulation with therapeutic response prediction ability. These cells express Cadherin 12 (CDH12, N-Cadherin 2), catenins, and other epithelial markers. CDH12-enriched tumors define patients with poor outcome following surgery with or without neoadjuvant chemotherapy (NAC), whereas CDH12-enriched tumors have a superior response to immune checkpoint therapy (ICT). Patient stratification by tumor CDH12 enrichment offered better prediction outcome than established bladder cancer subtypes. The CDH12 population resembles an undifferentiated state with chemoresistance. CDH12-enriched cells express PD-L1 and PD-L2 and co-localize with exhausted T-cells, possibly mediated through CD49a (ITGA1), likely explaining ICT efficacy in these tumors. This invention identifies a cancer cell population with a diametric response to major bladder cancer therapeutics, and provides a framework for designing biomarker-guided clinical trials.

### CROSS-REFERENCE TO RELATED APPLICATIONS

This application includes a claim of priority under 35 U.S.C. § 119(e) to U.S. provisional patent application No. 63/197,129, filed Jun. 4, 2021, the entirety of which is hereby incorporated by reference.

### STATEMENT REGARDING FEDERALLY SPONSORED RESEARCH OR DEVELOPMENT

This invention was made with government support under Grant No. CA143971 awarded by the National Institutes of Health. The Government has certain rights in the invention.

### FIELD OF INVENTION

This invention relates to therapeutics and prognostic markers in oncology, and especially in relation to cadherin expression in bladder tumor patients.

### BACKGROUND

Molecular subtyping of muscle-invasive bladder cancer (MIBC) has revolutionized the current conceptual thinking of MIBC pathogenesis. However, even the most recent consensus molecular classification systems do not provide compelling evidence for its use in clinical decision-making and is specifically lacking in predictions for therapeutic response. Emerging studies using single-cell RNA-sequencing to analyze MIBC have provided an initial understanding of intra-tumoral heterogeneity. However, these studies have focused on the tumor microenvironment, have been limited by relatively small cohort sizes, and have yet to provide a clearer path toward therapeutic decision-making.

Therefore, it is an objective of the present invention to provide comprehensive profiling at the single-cell level of MIBC epithelial and nonepithelial cells, which can help deconvolute molecular subtypes into their constituent parts.

It is another objective of the present invention to provide treatment methods, as well as prognostic and predictive tools, towards bladder cancer.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide methods of detections of one or more gene expression patterns in tumor cells, which can be used to identify or associate with respective phenotypes of the tumor cells, and/or to classify the tumor cells/cancer sample, and/or further provide prognosis or treatment selection for a subject. For example, a cadherin 12 (CDH12)-high phenotype of tumor cells or a cancer sample can be detected or characterized by an increased/higher expression in one or more or all genes in Gene Set 1; a CDH12-low phenotype of tumor cells or a cancer sample can be detected or characterized by an increased expression in one or more or all genes in Gene Set 2; a keratin 6A (KRT6A)-high phenotype of tumor cells or a cancer sample can be detected or characterized by an increased expression in one or more or all genes in Gene Set 3; a cell-cycle-related (cycling)-high phenotype can be detected or characterized by an increased expression in one or more or all genes in Gene Set 4; a uroplakins (UPK)-high phenotype can be detected or characterized by an increased expression in one or more or all genes in Gene Set 5; and a keratin 13-and-keratin 17 (KRT)-high phenotype can be detected or characterized by an increased expression in one or more or all genes in Gene Set 6 In further implementations, a detection includes detecting two or more phenotypes in tumor cells, thereby obtaining a ratio (relative occurrence/percentage) of one phenotype compared to another, or a presence of one phenotype and absence of one or more other phenotypes.

Additional embodiments provide methods of detections of one or more gene mutations (as an example of gene expression patterns) in tumor cells, which can be used to identify or associate with a CDH12-high phenotype or a CDH12-low phenotype, and/or to classify the tumor cells/cancer sample, and/or further provide prognosis or treatment selection for a subject. For example, a CDH12-high phenotype of tumor cells or a cancer sample can be detected or characterized by the presence of a gene mutation in at least one, at least two, at least three, at least four, at least five, at least six, or all seven of EIF4G3, ALAS1, NINL, NSD1, DFNA5, PABPC3, and TXNDC11. As another example, a CDH12-low phenotype of tumor cells or a cancer sample can be detected or characterized by the presence of a gene mutation in at least one, at least three, at least five, at least ten, or all 12 of ERBB2, FGFR3, PAPPA2, ASAP1, OCA2, NDC80, AP3D1, BAP1, KIFAP3, NOC3L, PAX7, and TNRC18.

Furthermore, methods are provided of detections in tumor cells of one or more gene expression patterns that are phenotypically most similar to the gene expression pattern in one undifferentiated/differentiated state of a normal cell, which can be used to classify the tumor cells/cancer sample, and/or further provide prognosis or treatment selection for a subject. For example, a gene expression pattern of latent time 0 detected in tumor cells can be detected or characterized by an increased/higher expression in one or more or all genes in Gene Set 7; a gene expression pattern of latent time 1 detected in tumor cells can be detected or characterized by an increased/higher expression in one or more or all genes in Gene Set 8; a gene expression pattern of latent time 2 detected in tumor cells can be detected or characterized by an increased/higher expression in one or more or all genes in Gene Set 9; a gene expression pattern of latent time 3 detected in tumor cells can be detected or characterized by an increased/higher expression in one or more or all genes in Gene Set 10; a gene expression pattern of latent time 4 detected in tumor cells can be detected or characterized by an increased/higher expression in one or more or all genes in Gene Set 11.

In various implementations, the increased/higher expression is relative to a reference, wherein the reference is the expression in one or more other phenotypes or expression patterns for each gene. In other embodiment, the increased/higher expression is relative to a reference, wherein the reference is the expression in all tumor cells (all phenotypes or expression patterns combined). In other embodiment, the increased/higher expression is relative to a reference, wherein the reference is the expression in tumor cells obtained from another subject.

Methods of providing prognosis, and/or treatment are further provided.

For example, detecting in tumor cells or a cancer sample obtained from a subject a CDH12-high phenotype, a majority (greater occurrence/percentage) of CDH12-high relative to other phenotypes, and/or a gene expression pattern of latent time 0 or latent time 1 indicates that the tumor cells or the subject is sensitive to an immunotherapy, e.g., an immune checkpoint inhibitor. Therefore, in some embodiments, a subject undergoing an immunotherapy is provided with a good survival prognosis and/or a good responsiveness prognosis if the subject is detected with a CDH12-high phenotype, a majority (greater occurrence/percentage) of CDH12-high relative to other phenotypes, and/or a gene expression pattern of latent time 0 or latent time 1. In some embodiments, a subject is selected to receive at least an immunotherapy, rather than a chemotherapy in the absence of an immunotherapy, if the subject is detected with a CDH12-high phenotype, a majority (greater occurrence/percentage) of CDH12-high relative to other phenotypes, and/or a gene expression pattern of latent time 0 or latent time 1.

As another example, detecting in tumor cells or a cancer sample obtained from a subject a CDH12-low phenotype, an absence or relative smaller occurrence/percentage CDH12-high relative to other phenotypes, and/or a gene expression pattern of latent time 4 or latent time 3 indicates that the tumor cells or the subject is sensitive to a chemotherapy (e.g., a neoadjuvant chemotherapy and/or an adjuvant chemotherapy) such as a platinum-based chemotherapy. Therefore, in some embodiments, a subject undergoing or having undergone a chemotherapy is provided with a good survival prognosis and/or a good responsiveness prognosis if the subject is detected with a CDH12-low phenotype, an absence or relative smaller occurrence/percentage CDH12-high relative to other phenotypes, and/or a gene expression pattern of latent time 4 or latent time 3. In some embodiments, a subject is selected to receive at least a chemotherapy if the subject is detected with a CDH12-low phenotype, an absence or relative smaller occurrence/percentage CDH12-high relative to other phenotypes, and/or a gene expression pattern of latent time 4 or latent time 3.

In various embodiments, the methods disclosed herein can be used for cancers such as bladder cancer, muscle invasive bladder cancer (MIBC), urothelial carcinoma, and others. In some embodiments, the cancer is a bladder cancer. In some embodiments, the cancer is a MIBC. In some embodiments, the cancer is a urothelial carcinoma.

Gene expression pattern may be performed by mRNA sequencing, preferably single-nuclei RNA sequence for determination/detection of expression levels, and/or by DNA sequencing for determination/detection of mutation.

Additional embodiments provide methods to use a combination of one or more Gene Sets provided herein as characteristics of each phenotype or expression pattern, as a starting point, to further detect differential gene expression patterns in one or more tumor samples obtained from patients before or after a specific therapy, optionally using one or more machines learning techniques, so as to identify a even more refined signature gene sets with differential expression pattern (upregulated or down-regulated) that is associated with the tumor samples and/or with the specific therapy.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive. This patent or application file contains at least one drawing executed in color. Copies of this patent or patent application publication with color drawing(s) will be provided by the Office upon request and payment of the necessary fee.

FIGS. 1A-1I depict discovery of a CDH12+ tumor cell population by single-nucleus sequencing. 1A, Workflow for single nucleus sequencing; MIBC—muscle invasive bladder cancer. 1B, Uniform manifold approximation and projection (UMAP) of all nuclei (71,832) in MIBC dataset colored by unsupervised clustering. 1C, Average gene expression per patient of marker genes for each cell type in FIG. 1B. 1D, UMAP of all epithelial nuclei (52,983) in MIBC dataset colored by epithelial population. 1E, Gene signature scores for published MIBC subtype gene sets. 1F, Uroepithelial differentiation-related marker gene expression in each epithelial population, where the dot size indicates the percent of cells within the subtype with non-zero expression of the respective gene. 1G, Gene-gene correlations partitioned into co-expression modules annotated for epithelial population enrichment. Gene ontology (GO) annotations are included with g:SCS multiple testing corrected p-values for hypergeometric testing. 1H, Activity scores for SCENIC regulons in each epithelial population. 1H, Gene signature scores for stem-cell and neuroendocrine differentiation gene sets.

FIGS. 2A-2F depict CDH12+ tumor population resembles characteristics of early undifferentiated urothelial cells and correlates with poor clinical outcome. 2A, UMAP of 12,819 uroepithelial nuclei obtained from histologically normal bladder and colored by unsupervised clustering. 2B, Uroepithelial differentiation-related marker gene expression. 2C, RNA velocity latent time trajectory in healthy bladder epithelial nuclei from a representative patient. 2D, RNA velocity-based latent time of the nuclei shown in FIG. 2C. 2E, Epithelial population density (top) and heatmap of uroepithelial marker gene expression (bottom) in nuclei from FIG. 2D ordered by increasing latent time. 2F, Epithelial population distribution across latent time for all normal samples combined (top row) or MIBC samples based on normal nearest neighbor analysis (middle row). Normal samples were combined by collating the latent times from velocity analyses performed on each of the 4 samples independently. Disease-specific survival of high-grade MIBC in TCGA stratified by gene signature scores derived from MIBC nuclei in the latent time intervals demarcated by the dashed lines (bottom row, log-rank test between top and bottom quartiles N=259).

FIGS. 3A-3E depict that high CDH12 scores predict chemoresistance and fibroblast activation. 3A, Average snSeq-derived signature scores in molecular subtypes of TCGA MIBC cases (N=259). Signatures highlighted in orange are shown in FIG. 3B. 3B, Disease specific survival of high-grade MIBC in TCGA stratified by snSeq population signatures (log-rank test between top and bottom quartiles, N=259). 3C, Tracking of 7 snSeq population signature scores in matched pre-chemo (left edge) and post-chemo samples (right edge) stratified by their pre-chemo CDH12 signature score (dark line indicates median of all samples shown as light lines, blue lines—low pre-chemo CDH12 score, red lines—high pre-chemo CDH12 score) (dashed line indicates p<0.001 for post-versus pre-chemo scores, Wilcoxon paired rank-sum test). 3D, GO term enrichment (hypergeometric overlap test) for genes up-regulated post-chemo in tumors with low or high CDH12 score in the pre-chemo setting. 3E, snSeq-derived receptor-ligand interactions significantly enriched between the CDH12 population and each fibroblast population.

FIGS. 4A-4G depict that post-chemo CDH12 score predicts favorable response to immune checkpoint therapy. 4A, PDL1 and PDL2 in matched pre-chemo and post-chemo samples (*—Wilcoxon paired two-sided rank-sum test p<0.05; n=65 for low CDH12, n=49 for high CDH12). Boxplots are drawn as the inter-quartile range (IQR) with a line indicating the median, and outliers defined as points that fall outside of the range demarcated by 1.5*IQR. 4B, PDL1 and PDL2 expression in snSeq tumor epithelial cells. 4C, Overall survival in IMvigor 210 Cohort 2 bladder tumors sequenced pre-chemo (top, N=100) or post-chemo (bottom, N=53) stratified by snSeq-derived population signature scores, or gene expression value (log-rank test, p=0 indicates p<0.001; * indicates gene expression). 4D, RECIST v1.1 response in bladder tumors profiled post-chemo stratified by CDH12 score quartile; progressive disease (PD), stable disease (SD), partial response (PR), complete response (CR) (*—Fisher exact test for PD vs PR/CR in quartile 1 vs quartile 4, N=51). 4E, Association of snSeq-derived signature scores, or consensus MIBC subtypes, with RECIST v1.1 response in the IMvigor 210 Cohort 2 cases shown in FIG. 4D (Fisher exact test, N=51). 4F, snSeq-derived receptor-ligand interactions significantly enriched between CDH12 population and each T-cell population. 4G, snSeq-derived receptor-ligand interaction potential of co-inhibitory signaling from epithelial populations to the CD8T population.

FIGS. 5A-5H depict that CDH12 tumor cells preferentially colocalize with T-cells expressing CD49a, PD-1, and LAG3. 5A, Schematic for topological analysis on the Visium spot hexagonal grid where the average expression of a gene is shown in a reference spot (gray) along with the average expression of the same gene in the spots located 1 spot away from the reference (red) or 2 spots away from the reference (orange) (top). Average expression of T-cell exhaustion and other immune markers surrounding spots enriched for each of 3 different Visium-derived epithelial signatures (bottom). * indicates p<0.05 using a Fisher exact test for testing the association of expression of a given gene with enrichment of a given epithelial score. 5B. Schematic of a MIBC tissue microarray (TMA) for multiplexed immunohistochemistry via CO-Detection by indEXing (CODEX). The CODEX panel consisted of 35 markers targeting epithelial, immune, and stromal cell types identified via snSeq analysis. 5C. Median spatial distance per TMA spot of KRT13⁺ (yellow) or CDH12⁺ (blue) epithelial cells to the nearest B-cell, CD4⁺ T-cell, CD8⁺ T-cell, macrophage, or fibroblast. *—Mann-Whitney, two-sided, p<0.05. n=36, 63, 34, 63, 18, 40, 40, 66, 41, 68 for each box from left to right. 5D. Voronoi diagrams of cellular neighborhoods (CN; top) and cell types (bottom). CN's were identified by k-means clustering the distribution of cell types neighboring each cell. Spots were chosen based on the number of cells belonging to each of the 5 epithelial cell enriched CN's. 5E. Cellular diversity measured by the Shannon entropy of the cell types composing each of 5 epithelial enriched CN's. *—Mann-Whitney, two-sided, p<0.05. n=42, 23, 63, 68, 67 for each box from left to right. 5F. Marker intensity enrichment on CD8⁺ T-cells residing within each CN, compared against CD8⁺ T-cells residing in any other CN. Only Wilcoxon (two-sided) p<0.05 are shown. 5G. Sample images from n=1 representative sample depicting a CD49a⁺ CD8⁺ T-cell (top), and PD-1⁺ CD8⁺ T-cell (bottom) in the immediate vicinity of CDH12⁺ epithelial cells in-situ. Scale bar—11 μm. 5H. Marker intensity enrichment on CDH12⁺ epithelial cells within each CDH12 enriched CN compared with CDH12⁻ epithelial cells within CN13 (left) or CDH12⁺ cells residing in any other CN (right). Only Wilcoxon (two-sided) p<0.05 are shown. Boxplots are drawn as the inter-quartile range (IQR) with a line indicating the median, and outliers defined as points that fall outside of the range demarcated by 1.5*IQR.

FIGS. 6A and 6B depict gene signatures derived from single-nuclei sequencing and spatial transcriptomics outperforms bulk-RNA sequencing-based consensus classifiers in predicting response to immune checkpoint therapy. 6A, Association of snSeq/visium-derived signature scores, or consensus MIBC subtypes, with RECIST v1.1 response in IMvigor 210 Cohort 2 (N=298, Fisher exact test). 6B, Flow chart for incorporating a CDH12 score into clinical decision making for treatment-naïve and chemoresistant tumors.

FIGS. 7A-7J depict a single nucleus sequencing of the MIBC tumor microenvironment. 7A, QC metrics for MIBC snSeq dataset where the blue horizontal lines represent the top and bottom 5th percentiles for the number of unique genes and total UMI or the 10% threshold for the UMI percent mitochondrial-coding genes. 7B, Scrublet scores for each of the histologically-normal bladder samples. 7C, snSeq population proportions in 25 muscle invasive bladder tumors, and the overall combined population proportions. 7D, Percent of patients analyzed that are represented in each of the unsupervised clusters using the single cell Variational Inference (scVI) model method. 7E, Average gene expression per patient of marker genes for each epithelial population in FIG. 1D. 7F, Epithelial population distribution for each patient analyzed. 7G, UMAP of fibroblasts (2,075 nuclei) from MIBC tumors colored by unsupervised clustering. 7H, Average gene expression per patient of marker genes for each fibroblast population in FIG. 7G. 7I, UMAP of immune cells (6,121 nuclei) from MIBC tumors colored by unsupervised clustering. 7I, Average gene expression per patient of marker genes for each immune population in FIG. 7I. Gene expression values shown as log(CP10k+1), heatmaps show average gene expression per cluster and z-scored within each patient.

FIG. 8 depicts immunohistochemistry validation of KRT13 and KRT17 expression in 4 tumors from MIBC cohort. Scale bars are 400 μm, 870 μm, and 10 μm in the left, middle and right columns, respectively.

FIG. 9 depicts immunohistochemistry validation of CDH12 and CDH18 expression in 4 tumors from MIBC cohort. Scale bars in the left column are shown with their respective lengths and scale bars in the right column are 10 μm.

FIGS. 10A-10E depict single nucleus sequencing of healthy bladders. 10A, Gene signature scores of co-expression modules identified in FIG. 1G separated by epithelial population. 10B, Epithelial populations (left, same as FIG. 1D) and ALDH1A1 expression in the MIBC epithelial nuclei (right). 10C, Normal bladder epithelial populations (left, same as FIG. 2A) and umbrella (middle) and basal (right) cell gene signature scores in 12,819 epithelial nuclei from histologically-normal bladders. 10D, Expression of genes commonly overexpressed in bladder cancers in MIBC versus normal bladder CDH12 populations. 10E, Density plots of the healthy bladder epithelial populations ordered by latent time in each of the 4 histologically-normal bladder tissues that were profiled.

FIGS. 11A-11C depict snSeq-derived gene signatures in NAC-treated tumors. 11A, snSeq-derived population signatures in pre-NAC samples per Genomic Subtyping Classifier subtype. (n=81 for luminal, n=59 for basal, n=45 for claudin-low, and n=38 for luminal-infiltrated (lumen-inf.) (* indicates two-sided Mann-Whitney p<=0.05. Boxplots are drawn as the inter-quartile range (IQR) with a line indicating the median, and outliers defined as points that fall outside of the range demarcated by 1.5*IQR.) 11B, Pathological downstaging of NAC-treated MIBC stratified by pre-NAC CDH12 score quartiles (log-rank test upper versus lower quartiles). 11C, Overall survival in NAC-treated MIBC stratified by snSeq-derived population signatures (log-rank test upper versus lower quartiles). Response was defined as pathologic downstaging (<pT2N0).

FIGS. 12A-12D depict survival prediction in IMvigor 210 by snSeq-derived gene signatures. 12A, Diagram showing cohort selection for IMvigor 210 analyses. The sample numbers indicate number of samples fitting those criteria for which sequencing data is available The top diagram shows the selection for the survival analyses and response predictions for all figures except FIG. 6A. The bottom diagram shows the selection for the response predictions in FIG. 6A. 12B, Overall survival in IMvigor 210 Cohort 2 bladder tumors sequenced pre-chemo (top, N=100) or post-chemo (bottom, N=53) stratified by snSeq-derived population signature scores (log-rank test between top and bottom quartiles; p=0 indicates p<0.001). 12C, QC metrics for Visium dataset where the blue horizontal lines show the cutoffs used for filtering spots. 12D, Visium-derived signature scores in snSeq UMAPs (top) and in-situ on MIBC visium samples (bottom). Stacked bar plots to the left of each visium sample show the corresponding snSeq population composition.

FIGS. 13A-13C depict CODEX cell type classification and niche identification. 13A, Example images showing nuclei (DAPI) with nuclear and membrane borders overlaid. Scale bar is 25 μm. 13B, CODEX marker intensity enrichment per cell subtype. Dot hue reflects the log 10 fold change, and the size of the dot indicates the Wilcoxon (two-sided) test p-value. 13C, CODEX marker intensity gating strategy used to gather training samples for cell subtyping. Cells were partitioned in a hierarchical fashion using combinations of cell lineage markers. When multiple markers are indicated on the same axis, these values were summed together for each cell. Plots outlined in a solid border were used for primary cell typing, and those outlined in a dashed border refer to intensity gates applied to primarily classified cells.

FIG. 14 depicts CODEX samples annotated by cell type. Every CODEX sample analyzed where each dot represents a cell centroid and is colored by the cell type.

FIGS. 15A-15C depict CODEX CDH12 and KRT13 staining and derivation of cellular niches (CN). 15A, Example images showing CDH12 and KRT13 staining on epithelial cells. Scale bar is 25 μm. 15B, Average area under the receiver operating characteristic curve (AUC) derived from logistic regression models fit on cellular neighbor profiles (percentage of each broad cell type immediately surrounding each cell) clustered into k clusters. The value of k was varied from 5 to 50 in increments of 5. A high average AUC indicates high predictability of each niche from the others. The vertical dotted line at k=20 indicates the number of cellular niches (CN) chosen for further analysis. 15C, Enrichment of subtypes assigned to each CN compared to any other CN. Dot hue and size reflect Fisher's exact test odds ratio and p-value, respectively.

FIG. 16 depicts CODEX samples annotated by cellular niche (CN). Every CODEX sample analyzed where each dot represents a cell centroid and is colored by the CN to which the cell belongs.

FIG. 17 depicts the mutation frequency (%) of each gene in the C3/CDH12-high epithelial population and in the CD/CDH12-low epithelial population. An algorithm calculates the C3 signature enrichment score on the TCGA MIBC samples, using the top 200 most upregulated genes in C3 versus other bladder tumor epithelial cells and the single sample Gene Set Enrichment Analysis tool. Samples in the top (C3 High) and bottom (C3 Low) quartile based on C3 scores are then compared for enrichments in gene level mutations using a chi-squared test (odds >1 and p-val <0.05). For example in this chart, ERBB2 is much more frequently mutated in the C3 Low epithelial population (about 16%) than in the C3 High epithelial population (about 4%); therefore, a new tumor or its epithelial cells (which may account for about 90% or more of the number of cells in the tumor) having a high amount of ERBB2 mutation, relative to a control, may indicate that this new tumor (or its epithelial cells) is a CDH12-low (or C3 Low) population. As another example, EIF4G3 is much more frequently mutated in C3 High epithelial population (about 9%) than in the C3 Low epithelial population (about 1%); therefore, a new tumor or its epithelial cells (which may account for about 90% or more of the number of cells in the tumor) having a high amount of EIF4G3 mutation, relative to a control, may indicate that this new tumor (or its epithelial cells) is a CDH12-high (or C3 High) population. These genes shown in FIG. 17 can then be used to develop a predictive model for progression. For example, a new tumor may be indicated to be “C3 High” (that is, CDH12-high) if it has one or more C3-high related mutations (e.g., 1, 2, 3, 4, 5, 6, or 7 C3-high related mutations) and zero “C3-low” related mutations. The predictive or prognostic features of a CDH12-high population and those of a CDH12-low population are exemplified in the Example section.

### BRIEF DESCRIPTION OF THE GENE SETS

Gene Set 1 depicts a list of 765 genes (by signature scores in a descending order, approximating log FC in a descending order) with largest positive values of log(expression Fold Change)>1.2 and FDR<0.1 in CDH12-expressing cancer epithelial cells, representing approximately most upregulated genes in the CDH12-expressing subtype, compared to all other subtypes combined. Accordingly, a CDH12-high phenotype can be identified with a gene expression pattern comprising an increased/higher expression in all or at least one or more of the 765 genes in Gene Set 1, relative to the expression in all other subtypes of cancer epithelial cells.

Gene Set 2 depicts a list of 124 genes with largest negative values of log FC, i.e., log FC<−0.8, (in a descending order of |log FC|), in CDH12-expressing cancer epithelial cells, representing most down-regulated genes in the CDH12-expressing subtype compared to all other subtypes combined. Accordingly, a CDH12-low phenotype can be identified with a gene expression pattern comprising an increased/higher expression in all or at least one or more of the 124 genes in Gene Set 2, relative to the expression in all other subtypes of cancer epithelial cells.

Gene Set 3 depicts a list of 46 genes (by signature scores in a descending order, approximating log FC in a descending order) with largest positive values of log FC>1.2 and FDR<0.1 in KRT6A-expressing cancer epithelial cells, representing approximately most upregulated genes in the KRT6A-expressing subtype, compared to all other subtypes combined. Accordingly, a KRT6A-high phenotype can be identified with a gene expression pattern comprising an increased/higher expression in all or at least one or more of the 46 genes in Gene Set 3, relative to the expression in all other subtypes of cancer epithelial cells.

Gene Set 4 depicts a list of 298 genes (by signature scores in a descending order, approximating log FC in a descending order) with largest positive values of log FC>1.2 and FDR>0.1 in cancer epithelial cells expressing cell-cycle-related genes (“cycling” subtype), representing approximately most upregulated genes in the cycling subtype, compared to all other subtypes combined. Accordingly, a cycling-high phenotype can be identified with a gene expression pattern comprising an increased/higher expression in all or at least one or more of the 298 genes in Gene Set 4, relative to the expression in all other subtypes of cancer epithelial cells.

Gene Set 5 depicts a list of 187 genes (by signature scores in a descending order, approximating log FC in a descending order) with largest positive values of log FC>1.2 and FDR<0.1 in UPK-expressing cancer epithelial cells, representing approximately most upregulated genes in the UPK subtype, compared to all other subtypes combined. Accordingly, a UPK-high phenotype can be identified with a gene expression pattern comprising an increased/higher expression in all or at least one or more of the 187 genes in Gene Set 5, relative to the expression in all other subtypes of cancer epithelial cells.

Gene Set 6 depicts a list of 419 genes (by signature scores in a descending order, approximating log FC in a descending order) with largest positive values of log FC>1.2 and FDR<0.1 in KRT13⁺/KRT17⁺ cancer epithelial cells (KRT-subtype), representing approximately most upregulated genes in the KRT subtype, compared to all other subtypes combined. Accordingly, a UPK-high phenotype can be identified with a gene expression pattern comprising an increased/higher expression in all or at least one or more of the 419 genes in Gene Set 6, relative to the expression in all other subtypes of cancer epithelial cells.

Gene Set 7 depicts a list of 178 genes (by signature scores in a descending order, approximating log FC in a descending order) with largest positive values of log FC>1.25 in previously untreated high-grade urothelial MIBC tumor samples in TCGA, exhibiting the gene expression pattern of latent “time 0” (most stem-like, i.e., uroepithelial undifferentiated phenotype) based on phenotypically most similar normal cells, representing approximately most upregulated genes in cancer cells with an expression pattern of latent time 0, compared to other cancer cells of other latent times. Accordingly, a latent-time-0 expression pattern can be identified with a gene expression pattern comprising an increased/higher expression in all or at least one or more of the 178 genes in Gene Set 7, relative to the expression in cancer cells of other latent times.

Gene Set 8 depicts a list of 47 genes (by signature scores in a descending order, approximating log FC in a descending order) with largest positive values of log FC>0.75 in previously untreated high-grade urothelial MIBC tumor samples in TCGA, exhibiting the gene expression pattern of latent “time 1” based on phenotypically most similar normal cells, (more differentiated than latent time 0 but less differentiated than latent time 2), representing approximately most upregulated genes in cancer cells with an expression pattern of latent time 1, compared to other cancer cells of other latent times. Accordingly, a latent-time-1 expression pattern can be identified with a gene expression pattern comprising an increased/higher expression in all or at least one or more of the 47 genes in Gene Set 8, relative to the expression in cancer cells of other latent times.

Gene Set 9 depicts a listing of 160 genes (by signature scores in a descending order, approximating log FC in a descending order) with largest positive values of log FC>1.65 in previously untreated high-grade urothelial MIBC tumor samples in TCGA, exhibiting the gene expression pattern of latent “time 2” based on phenotypically most similar normal cells, (more differentiated than latent time 1 but less differentiated than latent time 3), representing approximately most upregulated genes in cancer cells with an expression pattern of latent time 2, compared to other cancer cells of other latent times. Accordingly, a latent-time-2 expression pattern can be identified with a gene expression pattern comprising an increased/higher expression in all or at least one or more of the 160 genes in Gene Set 9, relative to the expression in cancer cells of other latent times.

Gene Set 10 depicts a list of 160 genes (by signature scores in a descending order, approximating log FC in a descending order) with largest positive values of log FC>1.35 in previously untreated high-grade urothelial MIBC tumor samples in TCGA, exhibiting the gene expression pattern of latent “time 3” based on phenotypically most similar normal cells, (more differentiated than latent time 2 but less differentiated than latent time 4), representing approximately the most upregulated genes in cancer cells with an expression pattern of latent time 3, compared to other cancer cells of other latent times. Accordingly, a latent-time-3 expression pattern can be identified with a gene expression pattern comprising an increased/higher expression in all or at least one or more of the 160 genes in Gene Set 10, relative to the expression in cancer cells of other latent times.

Gene Set 11 depicts a list of 190 genes (by signature scores in a descending order, approximating log FC in a descending order) with expression log FC>1.55 in previously untreated high-grade urothelial MIBC tumor samples in TCGA, exhibiting the gene expression pattern of latent “time 4” based on phenotypically most similar normal cells, (most uroepithelial differentiated, i.e., more differentiated than latent 3), representing approximately the most upregulated genes in cancer cells with an expression pattern of latent time 4, compared to other cancer cells of other latent times. Accordingly, a latent-time-4 expression pattern can be identified with a gene expression pattern comprising an increased/higher expression in all or at least one or more of the 190 genes in Gene Set 11, relative to the expression in cancer cells of other latent times.

Gene Set 12 depicts a list of signature genes used for ssGSEA analysis of bulk RNA-seq data for latent time 0.

Gene Set 13 depicts a list of signature genes used for ssGSEA analysis of bulk RNA-seq data for latent time 1.

Gene Set 14 depicts a list of signature genes used for ssGSEA analysis of bulk RNA-seq data for latent time 2.

Gene Set 15 depicts a list of signature genes used for ssGSEA analysis of bulk RNA-seq data for latent time 3.

Gene Set 16 depicts a list of signature genes used for ssGSEA analysis of bulk RNA-seq data for latent time 4.

Gene Set 17 depicts a list of signature genes used for ssGSEA analysis of bulk RNA-seq data for CDH12-expressing epithelial cells.

Gene Set 18 depicts a list of signature genes used for ssGSEA analysis of bulk RNA-seq data for KRT6A-expressing epithelial cells.

Gene Set 19 depicts a list of signature genes used for ssGSEA analysis of bulk RNA-seq data for UPK-expressing epithelial cells.

Gene Set 20 depicts a list of signature genes used for ssGSEA analysis of bulk RNA-seq data for KRT13-expressing epithelial cells.

Gene Set 21 depicts a list of signature genes used for ssGSEA analysis of bulk RNA-seq data for epithelial cells expressing cell cycle-related genes.

Gene Set 22 depicts a list of signature genes used for ssGSEA analysis of bulk RNA-seq data for antigen-presenting macrophages.

Gene Set 23 depicts a list of signature genes used for ssGSEA analysis of bulk RNA-seq data for activated B cells.

Gene Set 24 depicts a list of signature genes used for ssGSEA analysis of bulk RNA-seq data for dendritic cells.

Gene Set 25 depicts a list of signature genes used for ssGSEA analysis of bulk RNA-seq data for inflammatory macrophages.

Gene Set 26 depicts a list of signature genes used for ssGSEA analysis of bulk RNA-seq data for late activation CD8+ T cells.

Gene Set 27 depicts a list of signature genes used for ssGSEA analysis of bulk RNA-seq data for naïve T cells.

Gene Set 28 depicts a list of signature genes used for ssGSEA analysis of bulk RNA-seq data for plasma cells.

Gene Set 29 depicts a list of signature genes used for ssGSEA analysis of bulk RNA-seq data for Treg.

Gene Set 30 depicts a list of signature genes used for ssGSEA analysis of bulk RNA-seq data for smooth muscle α actin (ACTA2)-expressing fibroblasts.

Gene Set 31 depicts a list of signature genes used for ssGSEA analysis of bulk RNA-seq data for endothelial cells.

Gene Set 32 depicts a list of signature genes used for ssGSEA analysis of bulk RNA-seq data for fibroblast activation protein (FAP)-positive fibroblasts.

Gene Set 33 depicts a list of signature genes used for ssGSEA analysis of bulk RNA-seq data for PDGFRβ-expressing fibroblast.

Gene Set 34 depicts a list of signature genes used for ssGSEA analysis of bulk RNA-seq data for podoplanin (PDPN)-expressing fibroblast.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Sambrook and Russel, Molecular Cloning. A Laboratory Manual 4^{th} ed., Cold Spring Harbor Laboratory Press (Cold Spring Harbor, NY 2012) provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The bladder is a hollow organ in the pelvis with flexible, muscular walls, where the body stores urine before it leaves the body. The bladder wall has many layers, made up of different types of cells. The inside lining of the bladder is urothelium or transitional epithelium. Urine is carried from the kidneys to the bladder through tubes called ureters. When muscles in your bladder contract, they push urine out through a tube called the urethra.

A person with bladder cancer will have one or more tumors in his/her bladder. Muscle invasive bladder cancer (MIBC) is a cancer that spreads into the detrusor muscle of the bladder. The detrusor muscle is the thick muscle deep in the bladder wall. Transitional cell carcinoma (sometimes also called urothelial carcinoma) is cancer that forms in the cells of the urothelium, where most bladder cancers start. Symptoms of bladder cancer include hematuria (blood in the urine; often without pain), frequent an urgent need to pass urine, pain when passing urine, pain in the lower abdomen, and back pain.

The stage of bladder cancer can be identified from biopsies that are often done with transurethral resection of bladder tumor (TURBT), a procedure for tumor typing, staging and grading. The stages of bladder cancer are generally: i) Ta: tumor on the bladder lining that does not enter the muscle, ii) Tis: carcinoma in situ, looking like a reddish, velvety patch on the bladder lining, iii) T1: tumor goes through the bladder lining but does not reach the muscle layer, iv) T2: tumor grows into the muscle layer of the bladder, v) T3: tumor goes past the muscle layer into tissues around the bladder, and vi) T4: tumor has spread to nearby structures such as lymph nodes and the prostate in men or the vagina in females.

The term “expression levels” refers to a quantity reflected in or derivable from the gene or protein expression data, whether the data is directed to gene transcript accumulation or protein accumulation or protein synthesis rates, etc. In some embodiments, the term “expression level” refers to the amount of gene transcript accumulation; and in some embodiments, the term “expression level” refers to the amount of protein accumulation; and in other embodiments, the term “expression level” refers to the amount of either gene transcript accumulation or protein transcript accumulation.

In some embodiments, the cancer in the methods disclosed herein comprises bladder cancer, or urothelial cancer. In some embodiments, the bladder cancer is T4 stage. In some embodiments, the bladder cancer is T3 stage. In some embodiments, the bladder cancer is T2 stage. In some embodiments, the bladder cancer is T1 stage. In other embodiments, the cancer can be cervical carcinoma, colon cancer, rectal cancer, chordoma, lung cancer (e.g., non-small cell lung cancer), head and neck cancer, glioma, gliosarcoma, anaplastic astrocytoma, medulloblastoma, small cell lung carcinoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesothelioma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcoma, chondrosarcoma, osteogenic sarcoma, and leukemia. In some embodiments the cancer may be bladder cancer, lung cancer or head and neck cancer.

In some embodiments, the subject or patient is a human. In other embodiments, the subject or patient is a mammalian.

In this study, we perform the first comprehensive profiling of high-grade urothelial MIBCs using single-nucleus RNA-sequencing (snSeq) on 25 treatment-naïve patients, with surgery (TURBT/cystectomy) as their only treatment. We demonstrate the presence of a previously uncharacterized epithelial cell phenotype marked by high expression of Cadherin 12 (CDH12, N-Cadherin 2), catenins and other epithelial markers. We further show that this phenotype is present in multiple established molecular subtypes, demonstrating intra-subtype heterogeneity. We also find that CDH12-enriched tumors define patients with poor outcome following surgery with or without neoadjuvant chemotherapy, but superior outcome in the context of immune checkpoint therapy (ICT). Finally, using in-situ profiling we demonstrate that CDH12-enriched epithelial cells reside in distinct cellular niches that are enriched for exhausted CD8 T-cells, thus elucidating a possible mechanistic explanation for their ability to predict response to ICT. In various aspects, “CDH12-enriched” tumors, or referred to as “CDH12-high” tumors, have a plurality of biomarkers upregulated compared to “CDH12-poor” tumors, or alternatively referred to as “CDH-low” tumors, or compared to respective expression level in a control for each biomarker. In further aspects, one or more genes are more frequently mutated in CDH12-enriched or CDH12-high tumors, compared to in CDH12-poor or CDH12-low tumors, or compared to respective mutation rate (or percentage) in control for each of these genes. Alternatively, or in combination, one or more other genes are more frequently mutated in CDH12-poor or CDH12-low tumors.

### Tumor Cell Phenotypes/Subgroups

a. Phenotype Based on Gene Expression or Mutation Pattern in Tumor Cells

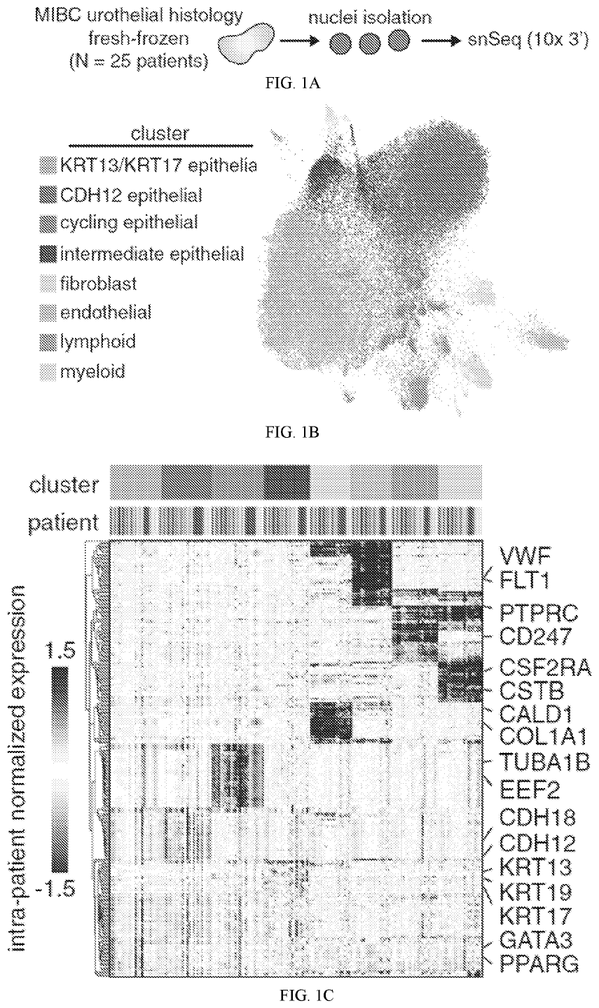

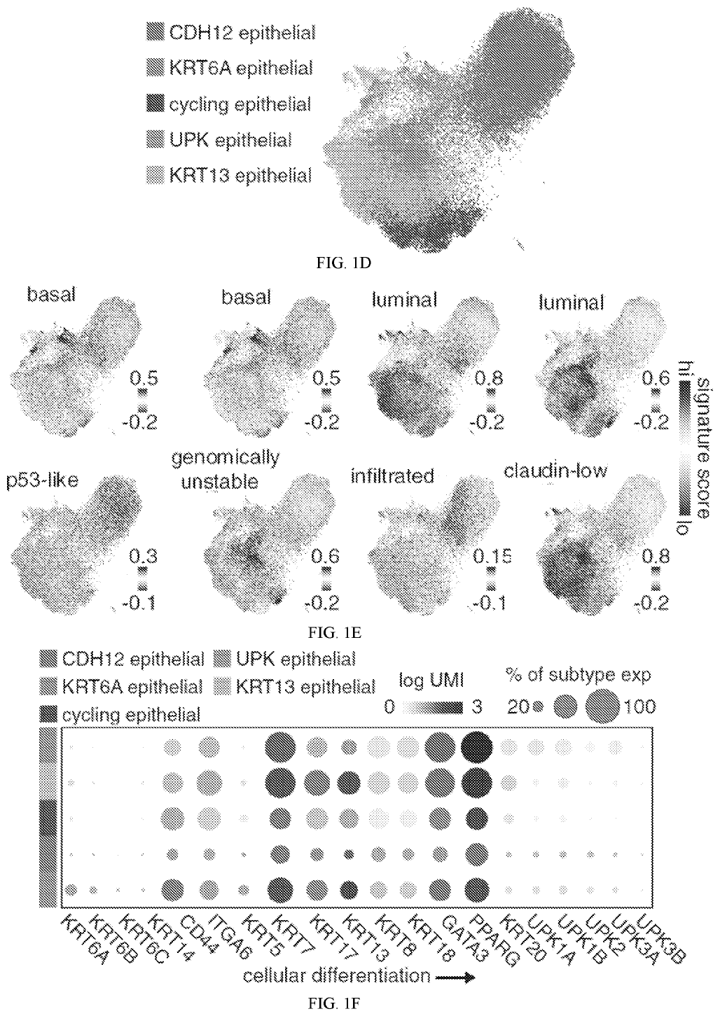

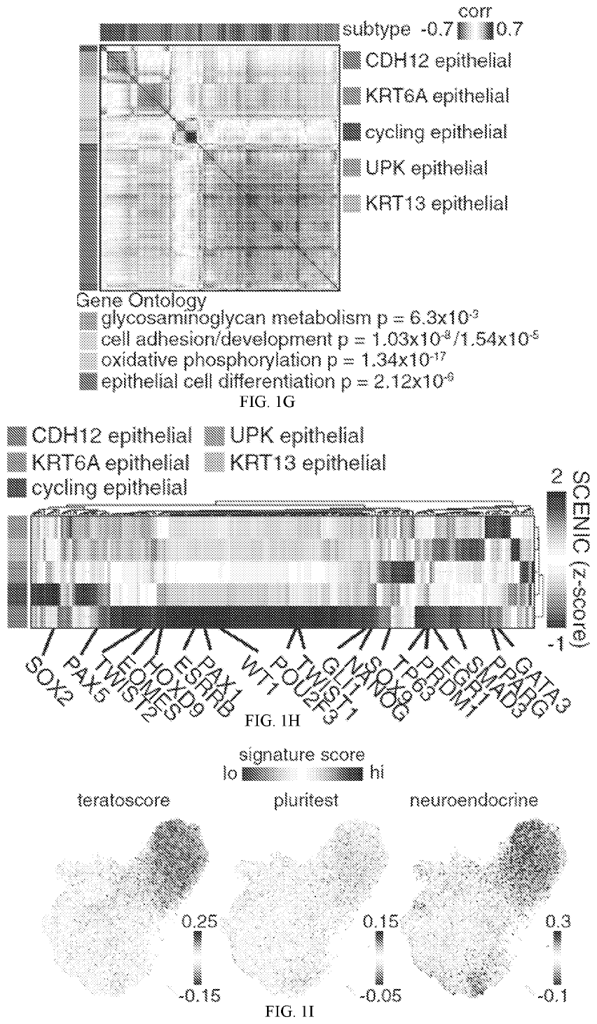

A tumor cell population has intratumoral heterogeneity. Various embodiments of the invention center around the different phenotypes (or clusters, subpopulations, or subtypes) exhibited in a population of tumor cells, wherein each phenotype is typically characterized by a distinct set of differentially expressed genes, or by a distinct set of differentially mutated genes, compared to other phenotypes within the tumor. For example, a bladder tumor may have a wide cellular composition, comprising epithelial cells, immune cells (such as lymphoids and myeloids), fibroblasts, and endothelial cells; and its epithelial cell subpopulation are discovered by the inventors to be composed of several epithelial cell clusters—one cluster with differential expression of CDH12, one cluster with differential expression of KRTi3 and KRT17, one cluster with differential expression of uroplakins (UPK), one cluster with differential expression of KRT6A, and one cluster with differential expression of cell-cycle-related genes. Each epithelial cluster can therefore be considered as a different phenotype, each having a distinct gene expression pattern characterized by the differentially expressed gene, identified above, along with other differentially expressed genes characteristic of the phenotype. See for example FIG. 1B. In instances where one cell type (e.g., epithelial cells) makes up for a majority (e.g., at least or about 90%, 80%, 75%, or 70%) of the tumor, the phenotypes of this one cell type may also represent the majority phenotypes of the tumor, and so we may refer to the tumor/cancer as having the different phenotypes.

In various embodiments, an N-cadherin 12 (CDH12) phenotype refers to a cell or cell population (or a subpopulation/subgroup of cells, relative to a bigger population/group with intra-group heterogeity) which expresses CDH12 and has a gene expression pattern wherein one or more genes in the list provided in Gene Set 1 are differentially expressed relative to a reference level for each gene. Specifically, the genes in the list of Gene Set 1 are “differentially expressed” with a log fold change (log(FC)) of at least 1.2; that is, their expression levels in the CDH12 phenotype are higher than respective expression levels in a reference. Therefore, in some embodiments, the CDH12 phenotype is also referred to as a “CDH12-high” phenotype for when the differentially expressed genes in at least Gene Set 1 are upregulated. A CDH12-high phenotype has a gene expression pattern wherein the one or more genes in Gene Set 1 are upregulated, i.e., having an increased/higher gene expression, relative to a reference level for respective gene.

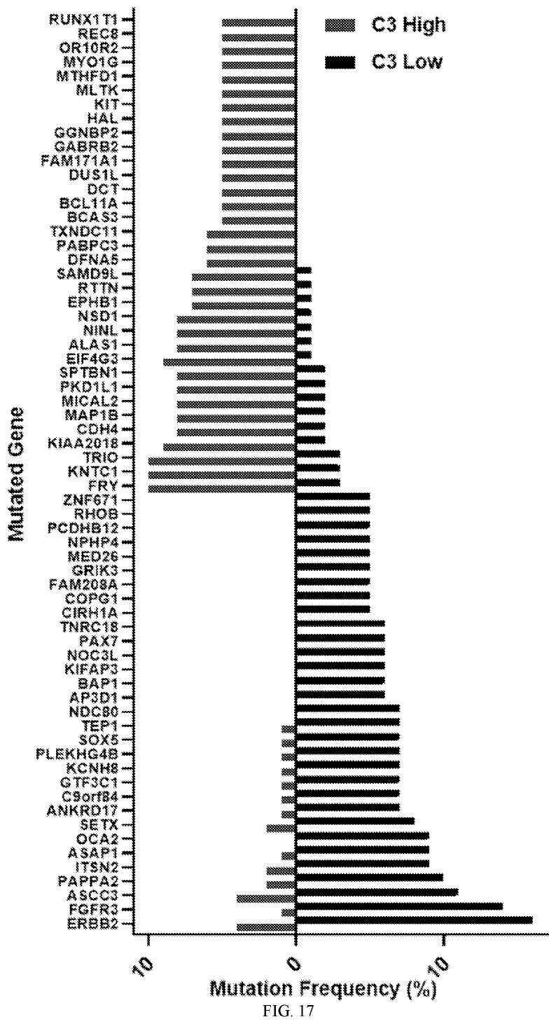

Gene Set 1 (as well as Gene Sets 2-11 for other phenotypes) names differentially expressed genes in a descending order by a score (e.g., the “C3” score in FIG. 17 and Example 1), which takes into account both the log(FC) and the false discovery rate (FDR).

In some embodiments, a CDH12-high phenotype has a gene expression pattern comprising an increased gene expression in all the genes in Gene Set 1.

In some embodiments, a CDH12-high phenotype has a gene expression pattern comprising an increased gene expression in at least 10 genes, preferably at least the first 10 genes, in Gene Set 1. In some embodiments, a CDH12-high phenotype has a gene expression pattern comprising an increased gene expression in at least 20 genes, preferably at least the first 20 genes, in Gene Set 1. In some embodiments, a CDH12-high phenotype has a gene expression pattern comprising an increased gene expression in at least 30 genes, preferably at least the first 30 genes, in Gene Set 1. In some embodiments, a CDH12-high phenotype has a gene expression pattern comprising an increased gene expression in at least 40 genes, preferably at least the first 40 genes, in Gene Set 1. In some embodiments, a CDH12-high phenotype has a gene expression pattern comprising an increased gene expression in at least 50 genes, preferably at least the first 50 genes, in Gene Set 1.

In some embodiments, a CDH12-high phenotype has a gene expression pattern comprising an increased gene expression in 1-10, 11-20, 21-30, 31-40, 41-50, 51-60, 61-70, 71-80, 81-90, 91-100, 101-150, 151-200, 201-300, 301-400, 401-500, 501-600, 601-700, or 701-765 genes, preferably the first 1-10, first 11-20, first 21-30, first 31-40, first 41-50, first 51-60, first 61-70, first 71-80, first 81-90, first 91-100, first 101-150, first 151-200, first 201-300, first 301-400, first 401-500, first 501-600, first 601-700, or first 701-765 genes, in Gene Set 1. In some embodiment, a CDH12-high phenotype has a gene expression pattern comprising an increased gene expression in one, two, three, or more (e.g., at least 10, 20, 30, 40, 50, 100, 200, 300, 500, 1000, 2000, or 3000), or all of the genes in the list titled “List of CDH12 subgroup of Epithelial Differential Gene Expression in Descending Order by Scores (log FC>0, FDR<0.05)” in the priority provisional application U.S. 63/197,129, which is incorporated by reference.

In contrast, a CDH12-low phenotype is, in various embodiments, one where the otherwise down-regulated genes in a CDH12 phenotype relative to other phenotypes (e.g., log FC<0) are actually upregulated compared to the other phenotypes. Therefore, a CDH12-low phenotype may have a gene expression pattern comprising an increased gene expression in one or more genes in Gene Set 2 relative to a reference. Gene Set 2 lists genes with the largest negative log FC values in the CDH12-high phenotype (in a descending order of |log FC|), therefore an increased/higher expression of one or more or all 124 genes in Gene Set 2 relative to other phenotypes (or a reference level) represents a gene expression pattern of the CDH12-low phenotype.

In some embodiments, a CDH12-low phenotype has a gene expression pattern comprising an increased gene expression in at least 10 genes, preferably at least the first 10 genes, in Gene Set 2. In some embodiments, a CDH12-low phenotype has a gene expression pattern comprising an increased gene expression in at least 20 genes, preferably at least the first 20 genes, in Gene Set 2. In some embodiments, a CDH12-low phenotype has a gene expression pattern comprising an increased gene expression in at least 30 genes, preferably at least the first 30 genes, in Gene Set 2. In some embodiments, a CDH12-low phenotype has a gene expression pattern comprising an increased gene expression in at least 40 genes, preferably at least the first 40 genes, in Gene Set 2. In some embodiments, a CDH12-low phenotype has a gene expression pattern comprising an increased gene expression in at least 50 genes, preferably at least the first 50 genes, in Gene Set 2.

In some embodiments, a CDH12-low phenotype has a gene expression pattern comprising an increased gene expression in 1-10, 11-20, 21-30, 31-40, 41-50, 51-60, 61-70, 71-80, 81-90, 91-100, 101-110, 111-120, or 120-124 genes, preferably the first 1-10, first 11-20, first 21-30, first 31-40, first 41-50, first 51-60, first 61-70, first 71-80, first 81-90, first 91-100, first 101-110, first 111-120, or first 121-124 genes, in the list provided in Gene Set 2.

In other embodiments, a CDH12-low phenotype has a gene expression pattern wherein the otherwise up-regulated genes in a CDH12 phenotype relative to other phenotypes (e.g., log FC>0) are actually downregulated compared to the other phenotypes. Therefore, a CDH12-low phenotype may have a gene expression pattern comprising a decreased/lower gene expression in one or more genes in Gene Set 1 relative to a reference. Gene Set 1 lists genes with the largest positive log FC values in the CDH12-high phenotype (in an approximately descending order of log FC>0), therefore a decreased/lower expression of one or more or all 765 genes in Gene Set 1 relative to other phenotypes (or to a reference level) represents a gene expression pattern of the CDH12-low phenotype. In additional embodiments, a CDH12-low phenotype has a gene expression pattern comprising a higher/increased gene expression in one or more genes in Gene Set 2 and a lower/decreased gene expression in one or more genes in Gene Set 1, relative to a reference.

Further embodiments provide using a gene mutation pattern as the expression pattern characteristics of a CDH12-high or a CDH12-low phenotype.

For example, a CDH12-high phenotype may have a gene expression pattern (or gene mutation pattern) wherein one or more genes are more frequently mutated than the mutation frequency in a reference sample or reference level (e.g., 0). For example, a CDH12-high phenotype has a gene mutation pattern comprising an increased gene mutation in at least 5, at least 10, at least 15, at least 20, at least 25, at least 30, all 34, or at least one of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to that in another phenotype (e.g., a CDH12-low phenotype). In some embodiments, the one or more genes more frequently mutated in a CDH12-high phenotype, relative to that in another phenotype, have odds >1 and p-value <0.05. In some embodiments, a CDH12-high phenotype has a gene mutation pattern comprising an increased gene mutation in at least 5 of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to that in another phenotype. In some embodiments, a CDH12-high phenotype has a gene mutation pattern comprising an increased gene mutation in at least 10 of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to that in another phenotype. In some embodiments, a CDH12-high phenotype has a gene mutation pattern comprising an increased gene mutation in at least 15 of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to that in another phenotype. In some embodiments, a CDH12-high phenotype has a gene mutation pattern comprising an increased gene mutation in at least 20 of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to that in another phenotype. In some embodiments, a CDH12-high phenotype has a gene mutation pattern comprising an increased gene mutation in at least 30 of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to that in another phenotype.

Preferably, a CDH12-high phenotype has a gene mutation pattern wherein EIF4G3, ALAS1, NINL, NSD1, DFNA5, PABPC3, and TXNDC11 are mutated; whereas these genes are not mutated in a CDH12-low phenotype. Therefore, the presence of mutation in one or more, or all, of EIF4G3, ALAS1, NINL, NSD1, DFNA5, PABPC3, and TXNDC11 are indicative of a CDH12-high phenotype in tumor cells (e.g., tumor CDH12-expression epithelial cells). In some embodiments, a CDH12-high phenotype has a gene expression pattern comprising a gene mutation in any one, two, three, four, five, six, or all seven of EIF4G3, ALAS1, NINL, NSD1, DFNA5, PABPC3, and TXNDC11. In some embodiments, detecting a CDH12-high phenotype detects a gene mutation in at least two of EIF4G3, ALAS1, NINL, NSD1, DFNA5, PABPC3, and TXNDC11. In some embodiments, detecting a CDH12-high phenotype detects a gene expression pattern comprising a gene mutation in at least three of EIF4G3, ALAS1, NINL, NSD1, DFNA5, PABPC3, and TXNDC11. In some embodiments, detecting a CDH12-high phenotype detects a gene expression pattern comprising a gene mutation in at least four of EIF4G3, ALAS1, NINL, NSD1, DFNA5, PABPC3, and TXNDC11. In some embodiments, detecting a CDH12-high phenotype detects a gene expression pattern comprising a gene mutation in at least five of EIF4G3, ALAS1, NINL, NSD1, DFNA5, PABPC3, and TXNDC11. In some embodiments, detecting a CDH12-high phenotype detects a gene expression pattern comprising a gene mutation in at least six of EIF4G3, ALAS1, NINL, NSD1, DFNA5, PABPC3, and TXNDC11. In some embodiments, detecting a CDH12-high phenotype detects a gene expression pattern comprising a gene mutation in all of EIF4G3, ALAS1, NINL, NSD1, DFNA5, PABPC3, and TXNDC11.

Other embodiments provide a CDH12-low phenotype has a gene expression pattern (or gene mutation pattern) wherein one or more genes are more frequently mutated than the mutation frequency in a reference sample or reference level. For example, a CDH12-low phenotype may have a gene expression pattern comprising an increased gene mutation in at least 5, at least 10, at least 15, at least 20, at least 25, at least 30, all 34, or at least one of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671, relative to that in another phenotype (e.g., a CDH12-high phenotype). In some embodiments, the one or more genes more frequently mutated in a CDH12-low phenotype have odds <1 and p-value <0.05. In some embodiments, a CDH12-low phenotype may have a gene expression pattern comprising an increased gene mutation in at least five of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671. In some embodiments, a CDH12-low phenotype may have a gene expression pattern comprising an increased gene mutation in at least ten of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671. In some embodiments, a CDH12-low phenotype may have a gene expression pattern comprising an increased gene mutation in at least 20 of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TN7RC18, C1RH1A, COPG1, FAM208A, GRIK3, MED26, NPHP4, PCDHB12, RHOB, and ZNF671. In some embodiments, a CDH12-low phenotype may have a gene expression pattern comprising an increased gene mutation in at least 30 of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671. In some embodiments, a CDH12-low phenotype may have a gene expression pattern comprising an increased gene mutation in all of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TN7RC18, C1RH1A, COPG1, FAM208A, GRIK3, MED26, NPHP4, PCDHB12, RHOB, and ZNF671.

Preferably, a CDH12-low phenotype has a gene mutation pattern wherein ERBB2, FGFR3, PAPPA2, ASAP1, OCA2, NDC80, AP3D, BAP1, KIFAP3, NOC3L, PAX7, and TN7RC18 are mutated; whereas these genes are not mutated in a CDH12-high phenotype. Therefore, the presence of mutation in one or more, or all, of ERBB2, FGFR3, PAPPA2, ASAP, OCA2, NDC80, AP3D1, BAP1, KIFAP3, NOC3L, PAX7, and TNRC18 are indicative of a CDH12-low phenotype in tumor cells (e.g., tumor epithelial cells). In some embodiments, a CDH12-low phenotype has a gene expression pattern comprising a gene mutation in any one, two, three, four, five, six, seven, eight, nine, ten, 11, or all 12 of ERBB2, FGFR3, PAPPA2, ASAP, OCA2, NDC80, AP3D1, BAP1, KIFAP3, NOC3L, PAX7, and TNRC18. In some embodiments, detecting a CDH12-low phenotype detects a gene mutation in at least two of ERBB2, FGFR3, PAPPA2, ASAP1, OCA2, NDC80, AP3D1, BAP1, KIFAP3, NOC3L, PAX7, and TNRC18. In some embodiments, detecting a CDH12-low phenotype detects a gene mutation in at least three of ERBB2, FGFR3, PAPPA2, ASAP1, OCA2, NDC80, AP3D1, BAP1, KIFAP3, NOC3L, PAX7, and TNRC18. In some embodiments, detecting a CDH12-low phenotype detects a gene mutation in at least four of ERBB2, FGFR3, PAPPA2, ASAP, OCA2, NDC80, AP3D1, BAP1, KIFAP3, NOC3L, PAX7, and TNRC18. In some embodiments, detecting a CDH12-low phenotype detects a gene mutation in at least five of ERBB2, FGFR3, PAPPA2, ASAP, OCA2, NDC80, AP3D, BAP1, KIFAP3, NOC3L, PAX7, and TNRC18. In some embodiments, detecting a CDH12-low phenotype detects a gene mutation in at least six of ERBB2, FGFR3, PAPPA2, ASAP1, OCA2, NDC80, AP3D1, BAP1, KIFAP3, NOC3L, PAX7, and TNRC18. In some embodiments, detecting a CDH12-low phenotype detects a gene mutation in at least seven of ERBB2, FGFR3, PAPPA2, ASAP1, OCA2, NDC80, AP3D1, BAP1, KIFAP3, NOC3L, PAX7, and TNRC18. In some embodiments, detecting a CDH12-low phenotype detects a gene mutation in at least eight of ERBB2, FGFR3, PAPPA2, ASAP1, OCA2, NDC80, AP3D1, BAP1, KIFAP3, NOC3L, PAX7, and TNRC18. In some embodiments, detecting a CDH12-low phenotype detects a gene mutation in at least nine of ERBB2, FGFR3, PAPPA2, ASAP, OCA2, NDC80, AP3D1, BAP1, KIFAP3, NOC3L, PAX7, and TNRC18. In some embodiments, detecting a CDH12-low phenotype detects a gene mutation in at least ten of ERBB2, FGFR3, PAPPA2, ASAP1, OCA2, NDC80, AP3D, BAP1, KIFAP3, NOC3L, PAX7, and TNRC18. In some embodiments, detecting a CDH12-low phenotype detects a gene mutation in all of ERBB2, FGFR3, PAPPA2, ASAP1, OCA2, NDC80, AP3D1, BAP1, KIFAP3, NOC3L, PAX7, and TNRC18.

Various embodiments provide that a keratin 6A (KRT6A) phenotype refers to a cell or cell population (or a subpopulation/subgroup of cells, relative to a bigger population/group with intra-group heterogeity) which expresses KRT6A and has a gene expression pattern wherein one or more genes in Gene Set 3 are differentially expressed relative to a reference level for each gene. Specifically, the genes in Gene Set 3 are “differentially expressed” with a log fold change (log(FC)) of at least 1.2; that is, their expression levels in the KRT6A phenotype are higher than respective expression levels in a reference; and so a KRT6A phenotype is also referred to as a “KRT6A-high” phenotype for when the differentially expressed genes are having an increased expression pattern. A KRT6A-high phenotype has a gene expression pattern wherein the one or more genes in the list provided in Gene Set 3 are upregulated, i.e., having an increased/higher gene expression, relative to a reference level for respective gene.

In some embodiments, a KRT6A phenotype, or KRT6A-high phenotype, has a gene expression pattern comprising an increased gene expression in all the genes in Gene Set 3. In some embodiments, a KRT6A phenotype, or KRT6A-high phenotype, has a gene expression pattern comprising an increased gene expression in at least 10 genes, preferably at least the first 10 genes, in Gene Set 3. In some embodiments, a KRT6A phenotype, or KRT6A-high phenotype, has a gene expression pattern comprising an increased gene expression in at least 20 genes, preferably at least the first 20 genes, in Gene Set 3. In some embodiments, a KRT6A phenotype, or KRT6A-high phenotype, has a gene expression pattern comprising an increased gene expression in at least 30 genes, preferably at least the first 30 genes, in Gene Set 3. In some embodiments, a KRT6A phenotype, or KRT6A-high phenotype, has a gene expression pattern comprising an increased gene expression in 1-10, 11-20, 21-30, 31-40, or 41-46 genes, preferably at least the first 1-10, first 11-20, first 21-30, first 31-40, or first 41-46 genes, in Gene Set 3. In further embodiments, a KRT6 phenotype, or KRT6A-high phenotype, has a gene expression pattern comprising an increased gene expression in one, two, three, or more (e.g., at least 10, 20, 30, 40, 50, 100, 200, 300, 400, or 500, preferably the first named ones), or all of the genes in the list titled “List of KRT6A subgroup of Epithelial Differential Gene Expression in Descending Order by Scores (log FC>0, FDR<0.05)” in the priority provisional application U.S. 63/197,129.

Various embodiments provide that a cell-cycle-related (cycling) phenotype refers to a cell or cell population (or a subpopulation/subgroup of cells, relative to a bigger population/group with intra-group heterogeity) which expresses markers such as KI67, SET and MYND domain containing 3 (SMYD3), centrosomal protein 192 (CEP192), AT-rich interaction domain 1B (ARID1B), Forkhead Box P1 (FOXP1), vascular endothelial growth factor A (VEGFA), and peroxisome proliferator-activated receptor gamma (PPARG), and which has a gene expression pattern wherein one or more genes in the list provided in Gene Set 4 are differentially expressed relative to a reference level for each gene. Specifically, the genes in Gene Set 4 are “differentially expressed” with a log fold change (log(FC)) of at least 1.2; that is, their expression levels in the cycling phenotype are higher than respective expression levels in a reference. The cycling phenotype is also referred to as a “cycling-high” phenotype for when the differentially expressed genes are having an increased expression pattern. So, a cycling-high phenotype has a gene expression pattern wherein the one or more genes in Gene Set 4 are upregulated, i.e., having an increased/higher gene expression, relative to a reference level for respective gene.

In some embodiments, a cycling phenotype, or cycling-high phenotype, has a gene expression pattern comprising an increased gene expression in all the genes in Gene Set 4. In some embodiments, a cycling phenotype, or cycling-high phenotype, has a gene expression pattern comprising an increased gene expression in at least 10 genes, preferably at least the first 10 genes, in Gene Set 4. In some embodiments, a cycling phenotype, or cycling-high phenotype, has a gene expression pattern comprising an increased gene expression in at least 20 genes, preferably at least the first 20 genes, in Gene Set 4. In some embodiments, a cycling phenotype, or cycling-high phenotype, has a gene expression pattern comprising an increased gene expression in at least 30 genes, preferably at least the first 30 genes, in Gene Set 4. In some embodiments, a cycling phenotype, or cycling-high phenotype, has a gene expression pattern comprising an increased gene expression in at least 40 genes, preferably at least the first 40 genes, in Gene Set 4. In some embodiments, a cycling phenotype, or cycling-high phenotype, has a gene expression pattern comprising an increased gene expression in at least 50 genes, preferably at least the first 50 genes, in Gene Set 4. In some embodiments, a cycling phenotype, or cycling-high phenotype, has a gene expression pattern comprising an increased gene expression in 1-10, 11-20, 21-30, 31-40, 41-50, 51-60, 61-70, 71-80, 81-90, 91-100, 101-150, 151-200, 201-250, or 251-298 genes, preferably the first 1-10, first 11-20, first 21-30, first 31-40, first 41-50, first 51-60, first 61-70, first 71-80, first 81-90, first 91-100 genes, first 101-150, first 151-200, first 201-250, or first 251-298 in Gene Set 4. In further embodiments, a cycling phenotype, or cycling-high phenotype, has a gene expression pattern comprising an increased gene expression in one, two, three, or more (e.g., at least 10, 20, 30, 40, 50, 100, 200, 300, 400, 500, or 1000 preferably the first named ones), or all of the genes in the list titled “List of cycling subgroup of Epithelial Differential Gene Expression in Descending Order by Scores (log FC>0, FDR<0.05)” in the priority provisional application U.S. 63/197,129.

Various embodiments provide that a UPK phenotype refers to a cell or cell population (or a subpopulation/subgroup of cells, relative to a bigger population/group with intra-group heterogeity) which expresses UPK and has a gene expression pattern wherein one or more genes in the list provided in Gene Set 5 are differentially expressed relative to a reference level for each gene. Specifically, the genes in Gene Set 5 are “differentially expressed” with a log fold change (log(FC)) of at least 1.2; that is, their expression levels in the UPK phenotype are higher than respective expression levels in a reference. The UPK phenotype is also referred to as a “UPK-high” phenotype for when the differentially expressed genes are having an increased expression pattern. So, a UPK-high phenotype has a gene expression pattern wherein the one or more genes in Gene Set 5 are upregulated, i.e., having an increased/higher gene expression, relative to a reference level for respective gene.

In some embodiments, a UPK phenotype, or UPK-high phenotype, has a gene expression pattern comprising an increased gene expression in all the genes in Gene Set 5. In some embodiments, a UPK phenotype, or UPK-high phenotype, has a gene expression pattern comprising an increased gene expression in at least 10 genes, preferably at least the first 10 genes, in Gene Set 5. In some embodiments, a UPK phenotype, or UPK-high phenotype, has a gene expression pattern comprising an increased gene expression in at least 20 genes, preferably at least the first 20 genes, in Gene Set 5. In some embodiments, a UPK phenotype, or UPK-high phenotype, has a gene expression pattern comprising an increased gene expression in at least 30 genes, preferably at least the first 30 genes, in Gene Set 5. In some embodiments, a UPK phenotype, or UPK-high phenotype, has a gene expression pattern comprising an increased gene expression in at least 40 genes, preferably at least the first 40 genes, in Gene Set 5. In some embodiments, a UPK phenotype, or UPK-high phenotype, has a gene expression pattern comprising an increased gene expression in at least 50 genes, preferably at least the first 50 genes, in Gene Set 5. In some embodiments, a UPK phenotype, or UPK-high phenotype, has a gene expression pattern comprising an increased gene expression in 1-10, 11-20, 21-30, 31-40, 41-50, 51-60, 61-70, 71-80, 81-90, 91-100, 101-150, or 151-187 genes, preferably the first 1-10, first 11-20, first 21-30, first 31-40, first 41-50, first 51-60, first 61-70, first 71-80, first 81-90, first 91-100, first 101-150, or first 151-187 genes, in Gene Set 5. In further embodiments, a UPK phenotype, or UPK-high phenotype, has a gene expression pattern comprising an increased gene expression in one, two, three, or more (e.g., at least 10, 20, 30, 40, 50, 100, 200, 300, 400, 500, or 1000, preferably the first named ones), or all of the genes in the list titled “List of UPK subgroup of Epithelial Differential Gene Expression in Descending Order by Scores (log FC>0, FDR<0.05)” in the priority provisional application U.S. 63/197,129.

Various embodiments provide that a KRT phenotype refers to a cell or cell population (or a subpopulation/subgroup of cells, relative to a bigger population/group with intra-group heterogeity) which expresses KRT13 and KRT17 and has a gene expression pattern wherein one or more genes in the list provided in Gene Set 6 are differentially expressed relative to a reference level for each gene. Specifically, the genes in Gene Set 6 are “differentially expressed” with a log fold change (log(FC)) of at least 1.2; that is, their expression levels in the KRT phenotype are higher than respective expression levels in a reference. The KRT phenotype is also referred to as a “KRT-high” phenotype for when the differentially expressed genes are having an increased expression pattern. So, a KRT-high phenotype has a gene expression pattern wherein the one or more genes in Gene Set 6 are upregulated, i.e., having an increased/higher gene expression, relative to a reference level for respective gene.

In some embodiments, a KRT phenotype, or KRT-high phenotype, has a gene expression pattern comprising an increased gene expression in all the genes in Gene Set 6. In some embodiments, a KRT phenotype, or KRT-high phenotype, has a gene expression pattern comprising an increased gene expression in at least 10 genes, preferably at least the first 10 genes, in Gene Set 6. In some embodiments, a KRT phenotype, or KRT-high phenotype, has a gene expression pattern comprising an increased gene expression in at least 20 genes, preferably at least the first 20 genes, in Gene Set 6. In some embodiments, a KRT phenotype, or KRT-high phenotype, has a gene expression pattern comprising an increased gene expression in at least 30 genes, preferably at least the first 30 genes, in Gene Set 6. In some embodiments, a KRT phenotype, or KRT-high phenotype, has a gene expression pattern comprising an increased gene expression in at least 40 genes, preferably at least the first 40 genes, in Gene Set 6. In some embodiments, a KRT phenotype, or KRT-high phenotype, has a gene expression pattern comprising an increased gene expression in at least 50 genes, preferably at least the first 50 genes, in Gene Set 6. In some embodiments, a KRT phenotype, or KRT-high phenotype, has a gene expression pattern comprising an increased gene expression in 1-10, 11-20, 21-30, 31-40, 41-50, 51-60, 61-70, 71-80, 81-90, 91-100, 101-150, 151-200, 201-250, 251-300, 301-350, 351-400, or 401-419 genes, preferably the first 1-10, first 11-20, first 21-30, first 31-40, first 41-50, first 51-60, first 61-70, first 71-80, first 81-90, first 91-100 genes, first 101-150, first 151-200, first 201-250, first 251-300, first 301-350, first 351-400, or first 401-419 in Gene Set 6. In further embodiments, a KRT phenotype, or KRT-high phenotype, has a gene expression pattern comprising an increased gene expression in one, two, three, or more (e.g., at least 10, 20, 30, 40, 50, 100, 200, 300, 400, 500, 1000, 2000, or 3000, preferably the first named ones), or all of the genes in the list titled “List of KRT13 subgroup of Epithelial Differential Gene Expression in Descending Order by Scores (log FC>0, FDR<0.05)” in the priority provisional application U.S. 63/197,129.

Additional embodiments provide that a tumor sample can have a CDH12-high (or C3-high) phenotype with a gene expression pattern comprising an increased gene mutation frequency in one or more genes indicated so in FIG. 17, e.g., one or more of RUNX1T1, REC8, OR10R2, MYO1G, MTHFD1, ML TK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level (e.g., those in a C3-low phenotype, or zero). In various embodiments, the gene expression pattern comprises an increased gene mutation frequency in two or more of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level. In various embodiments, the gene expression pattern comprises an increased gene mutation frequency in five or more of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level. In various embodiments, the gene expression pattern comprises an increased gene mutation frequency in ten or more of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level. In various embodiments, the gene expression pattern comprises an increased gene mutation frequency in 15 or more of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level. In various embodiments, the gene expression pattern comprises an increased gene mutation frequency in 20 or more of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level. In various embodiments, the gene expression pattern comprises an increased gene mutation frequency in 25 or more of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level. In various embodiments, the gene expression pattern comprises an increased gene mutation frequency in all of RUNX1T1, REC8, OR10R2, MYO1G, MTHFD1, MLTK, KIT, HAL, GGNBP2, GABRB2, FAM171A1, DUS1L, DCT, BCL11A, BCAS3, TXNDC11, PABPC3, DFNA5, SAMD9L, RTTN, EPHB1, NSDA, NINL, ALAS1, EIF4G3, SPTBN1, PKD1L1, MICAL2, MAP1B, CDH4, KIAA2018, TRIO, KNTC1, and FRY, relative to each gene's reference level.

In other embodiments, a tumor sample can have a CDH12-low (or C3) phenotype with a gene expression pattern comprising an increased gene mutation frequency in one or more genes indicated so in FIG. 17, e.g., one or more of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TN7RC18, C1RH1A, COPG1, FAM208A, GRIK3, MED26, NPHP4, PCDHB12, RHOB, and ZNF671, relative to each gene's reference level (e.g., those in a C3-high phenotype). In various embodiments, the gene expression pattern comprises an increased gene mutation frequency in two or more of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TN7RC18, C1RH1A, COPG1, FAM208A, GRIK3, MED26, NPHP4, PCDHB12, RHOB, and ZNF671, relative to each gene's reference level. In various embodiments, the gene expression pattern comprises an increased gene mutation frequency in five or more genes of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671, relative to each gene's reference level. In various embodiments, the gene expression pattern comprises an increased gene mutation frequency in 10 or more genes of ERBB2, FGFR3, ASCC3, PAPPA2, ITSN2, ASAP1, OCA2, SETX ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671, relative to each gene's reference level. In various embodiments, the gene expression pattern comprises an increased gene mutation frequency in 20 or more genes of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TN7RC18, C1RH1A, COPG1, FAM208A, GRIK3, MED26, NPHP4, PCDHB12, RHOB, and ZNF671, relative to each gene's reference level. In various embodiments, the gene expression pattern comprises an increased gene mutation frequency in 25 or more genes of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671, relative to each gene's reference level. In various embodiments, the gene expression pattern comprises an increased gene mutation frequency in all of ERBB2, FGFR3, ASCC3, PAPPA2, ITSN2, ASAP1, OCA2, SETX, ANKRD17, C9orf84, GTF3C1, KCNH8, PLEKHG4B, SOX5, TEP1, NDS80, AP3D1, BAP1, KIFAP3, NOC3L, PAX7, TNRC18, C1RH1A, COPG1, FAM208A, GRIK3, MED26, NPHP4, PCDHB12, RHOB, and ZNF671, relative to each gene's reference level.

b. Phenotype Based on Tumor Cells' Gene Expression Pattern that Phenotypically Mimicks A Undifferentiated/Differentiated State of Normal Cells

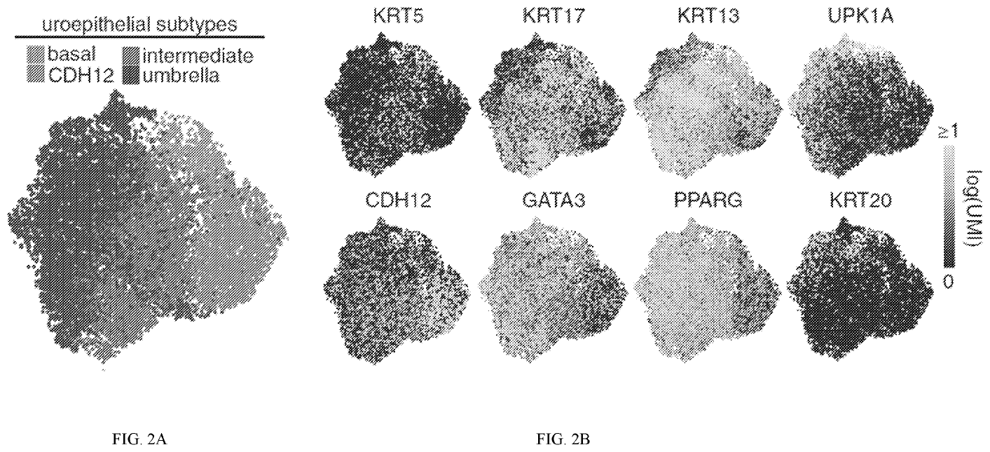

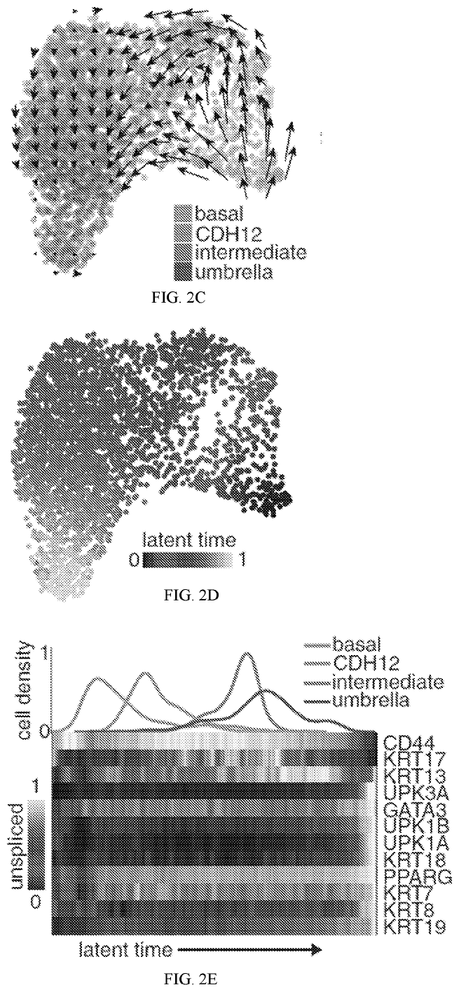

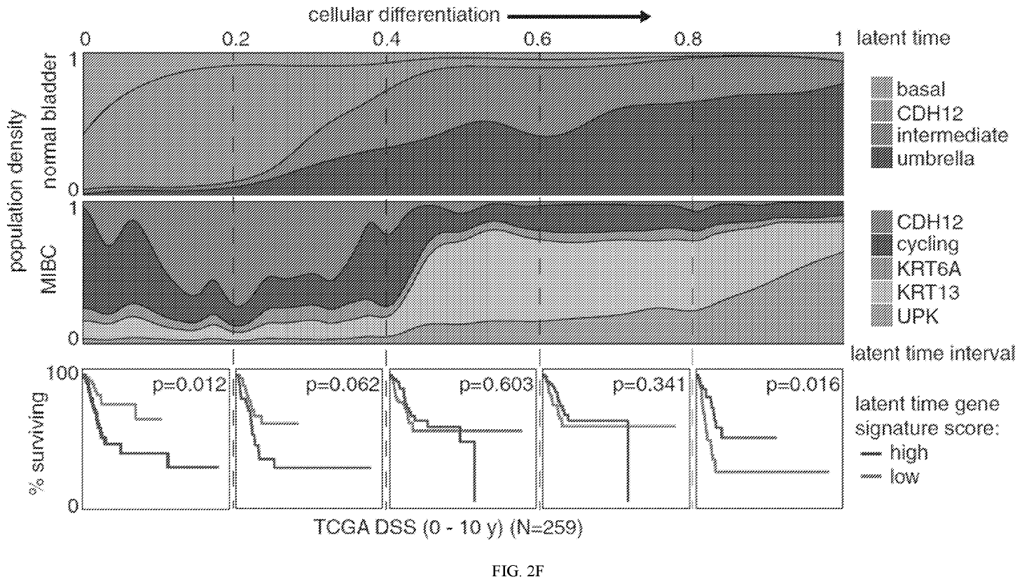

Various embodiments provide that the different phenotypes of a tumor also resemble, in terms of a gene expression pattern, the characteristics of an undifferentiated cellular state or a differentiated cellular state; and so the different phenotypes of a cancer may also be mapped to correspond with different points on a “differentiation” time scale, e.g., a progression trajectory from a most undifferentiated, least differentiated state to a most differentiated, least undifferentiated state. See for example, FIG. 1F, 2C. For example, in RNA velocity analysis, the expression ratio based on intron versus exon of a normal cell (non-cancerous cell) can infer a latent time of the normal cell (coined “normal latent time”); wherein an earlier latent time represents a more undifferentiated state, and a later latent time represents a more differentiated state. See for example FIG. 2E, 2F. A normal cell is also called the “nearest normal cell neighbor” to a tumor cell if the tumor cell's overall gene expression pattern is most similar to that normal cell (and not as similar to other normal cells on the latent time scale). The tumor cell therefore gets assigned a latent time that corresponds to the normal latent time of its “nearest normal cell neighbor.” For example, arbitrary numbers 0 and 4 may represent the most undifferentiated (least differentiated) latent time and the most differentiated (least undifferentiated) latent time, respectively, on a latent time scale. And latent time 1 is more differentiated than latent time 0 and less differentiated than latent time 2. So a series of 0, 1, 2, 3, and 4 indicates a temporal range from early to late latent time, or from a most stem-like, “undifferentiated” state to a differentiated state. As such, a tumor phenotype may also be characterized by the gene expression pattern of a latent time, and the inventors have identified a distinct set of differentially expressed genes for each latent time. This tumor phenotyping based on tumor cells' gene expression pattern of a specific latent time is an alternative characteristic to, or another characteristic combinable with, the distinct differentially expressed/mutated gene set by CDH12/KRT6A/cycline/UPK/KRT clustering described above.

A tumor cell having a gene expression pattern of “latent time 0” comprises one or more differentially expressed genes as listed in Gene Set 7 relative to a reference level for each gene. Specifically, the genes in the list of Gene Set 7 are differentially expressed with a log FC of at least 1.25; that is, their expression levels at the “latent time 0” are higher than respective expression levels in a reference. As such, a gene expression pattern of “latent time 0” has an increased/higher gene expression in one or more genes in the list provided in Gene Set 7 (e.g., relative to the expression in other latent times).

In some embodiments, a “latent time 0” gene expression pattern comprises an increased gene expression in all the genes in the list provided in Gene Set 7. In some embodiments, a “latent time 0” gene expression pattern has a gene expression pattern comprising an increased gene expression in at least 20 genes, preferably at least the first 20 genes, in the list provided in Gene Set 7. In some embodiments, a “latent time 0” gene expression pattern has a gene expression pattern comprising an increased gene expression in at least 50 genes, preferably at least the first 50 genes, in the list provided in Gene Set 7. In some embodiments, a “latent time 0” gene expression pattern has a gene expression pattern comprising an increased gene expression in at least 100 genes, preferably at least the first 100 genes, in the list provided in Gene Set 7. In some embodiments, a “latent time 0” gene expression pattern has a gene expression pattern comprising an increased gene expression in at least 150 genes, preferably at least the first 150 genes, in the list provided in Gene Set 7. In some embodiments, a “latent time 0” gene expression pattern has a gene expression pattern comprising an increased gene expression in 1-10, 11-20, 21-30, 31-40, 41-50, 51-60, 61-70, 71-80, 81-90, 91-100, 101-120, 121-140, 141-160, or 161-178 genes, preferably the first 1-10, first 11-20, first 21-30, first 31-40, first 41-50, first 51-60, first 61-70, first 71-80, first 81-90, first 91-100 genes, first 101-120, first 121-140, first 141-160, or first 161-178 genes in the list provided in Gene Set 7.

A gene expression pattern of “latent time 1” comprises one or more differentially expressed genes as listed in Gene Set 8 relative to a reference level for each gene. Specifically, the genes in the list of Gene Set 8 are differentially expressed with a log FC of at least 0.75; that is, their expression levels at the “latent time 1” compared to respective expression levels in a reference has a fold change of at least 2^{0.75}, i.e., a fold change greater than 1.68, being higher than respective expression levels in a reference. As such, a gene expression pattern of “latent time 1” has an increased/higher gene expression in one or more genes in the list provided in Gene Set 8.

In some embodiments, a “latent time 1” gene expression pattern comprises an increased gene expression in all the genes in the list provided in Gene Set 8. In some embodiments, a “latent time 1” gene expression pattern has a gene expression pattern comprising an increased gene expression in at least 10 genes, preferably at least the first 10 genes, in the list provided in Gene Set 8. In some embodiments, a “latent time 1” gene expression pattern has a gene expression pattern comprising an increased gene expression in at least 20 genes, preferably at least the first 20 genes, in the list provided in Gene Set 8. In some embodiments, a “latent time 1” gene expression pattern has a gene expression pattern comprising an increased gene expression in at least 30 genes, preferably at least the first 30 genes, in the list provided in Gene Set 8. In some embodiments, a “latent time 1” gene expression pattern has a gene expression pattern comprising an increased gene expression in at least 40 genes, preferably at least the first 40 genes, in the list provided in Gene Set 8. In some embodiments, a “latent time 1” gene expression pattern has a gene expression pattern comprising an increased gene expression in 1-10, 11-20, 21-30, 31-40, or 41-47 genes, preferably the first 1-10, first 11-20, first 21-30, first 31-40, or first 41-47 genes, in the list provided in Gene Set 8.

A gene expression pattern of “latent time 2” comprises one or more differentially expressed genes as listed in Gene Set 9 relative to a reference level for each gene. Specifically, the genes in the list of Gene Set 9 are differentially expressed with a log FC of at least 1.65; that is, their expression levels at the “latent time 2” compared to respective expression levels in a reference has a fold change of at least 2^{1.65}, i.e., a fold change greater than 3.13, being higher than respective expression levels in a reference. As such, a gene expression pattern of “latent time 2” has an increased/higher gene expression in one or more genes in the list provided in Gene Set 9.

In some embodiments, a “latent time 2” gene expression pattern comprises an increased gene expression in all the genes in the list provided in Gene Set 9. In some embodiments, a “latent time 2” gene expression pattern has a gene expression pattern comprising an increased gene expression in at least 20 genes, preferably at least the first 20 genes, in the list provided in Gene Set 9. In some embodiments, a “latent time 2” gene expression pattern has a gene expression pattern comprising an increased gene expression in at least 50 genes, preferably at least the first 50 genes, in the list provided in Gene Set 9. In some embodiments, a “latent time 2” gene expression pattern has a gene expression pattern comprising an increased gene expression in at least 100 genes, preferably at least the first 100 genes, in the list provided in Gene Set 9. In some embodiments, a “latent time 2” gene expression pattern has a gene expression pattern comprising an increased gene expression in at least 150 genes, preferably at least the first 150 genes, in the list provided in Gene Set 9. In some embodiments, a “latent time 2” gene expression pattern has a gene expression pattern comprising an increased gene expression in 1-10, 11-20, 21-30, 31-40, 41-50, 51-60, 61-70, 71-80, 81-90, 91-100, 101-120, 121-140, or 141-160 genes, preferably the first 1-10, first 11-20, first 21-30, first 31-40, first 41-50, first 51-60, first 61-70, first 71-80, first 81-90, first 91-100 genes, first 101-120, first 121-140, or first 141-160 genes in the list provided in Gene Set 9.

A gene expression pattern of “latent time 3” comprises one or more differentially expressed genes as listed in Gene Set 10 relative to a reference level for each gene. Specifically, the genes in the list of Gene Set 10 are differentially expressed with a log FC of at least 1.35; that is, their expression levels at the “latent time 3” compared to respective expression levels in a reference has a fold change of at least 2^{1.35}, i.e., a fold change greater than 2.54, being higher than respective expression levels in a reference. As such, a gene expression pattern of “latent time 3” has an increased/higher gene expression in one or more genes in the list provided in Gene Set 10.

In some embodiments, a “latent time 3” gene expression pattern comprises an increased gene expression in all the genes in the list provided in Gene Set 10. In some embodiments, a “latent time 3” gene expression pattern has a gene expression pattern comprising an increased gene expression in at least 20 genes, preferably at least the first 20 genes, in the list provided in Gene Set 10. In some embodiments, a “latent time 3” gene expression pattern has a gene expression pattern comprising an increased gene expression in at least 50 genes, preferably at least the first 50 genes, in the list provided in Gene Set 10. In some embodiments, a “latent time 3” gene expression pattern has a gene expression pattern comprising an increased gene expression in at least 100 genes, preferably at least the first 100 genes, in the list provided in Gene Set 10. In some embodiments, a “latent time 3” gene expression pattern has a gene expression pattern comprising an increased gene expression in at least 150 genes, preferably at least the first 150 genes, in the list provided in Gene Set 10. In some embodiments, a “latent time 3” gene expression pattern has a gene expression pattern comprising an increased gene expression in 1-10, 11-20, 21-30, 31-40, 41-50, 51-60, 61-70, 71-80, 81-90, 91-100, 101-120, 121-140, or 141-160 genes, preferably the first 1-10, first 11-20, first 21-30, first 31-40, first 41-50, first 51-60, first 61-70, first 71-80, first 81-90, first 91-100 genes, first 101-120, first 121-140, or first 141-160 genes in the list provided in Gene Set 10.

A gene expression pattern of “latent time 4” comprises one or more differentially expressed genes as listed in Gene Set 11 relative to a reference level for each gene. Specifically, the genes in the list of Gene Set 11 are differentially expressed with a log FC of at least 1.35; that is, their expression levels at the “latent time 3” compared to respective expression levels in a reference has a fold change of at least 2^{1.35}, i.e., a fold change greater than 2.54, being higher than respective expression levels in a reference. As such, a gene expression pattern of “latent time 4” has an increased/higher gene expression in one or more genes in the list provided in Gene Set 11.

In some embodiments, a “latent time 4” gene expression pattern comprises an increased gene expression in all the genes in the list provided in Gene Set 11. In some embodiments, a “latent time 4” gene expression pattern has a gene expression pattern comprising an increased gene expression in at least 20 genes, preferably at least the first 20 genes, in the list provided in Gene Set 11. In some embodiments, a “latent time 4” gene expression pattern has a gene expression pattern comprising an increased gene expression in at least 50 genes, preferably at least the first 50 genes, in the list provided in Gene Set 11. In some embodiments, a “latent time 4” gene expression pattern has a gene expression pattern comprising an increased gene expression in at least 100 genes, preferably at least the first 100 genes, in the list provided in Gene Set 11. In some embodiments, a “latent time 4” gene expression pattern has a gene expression pattern comprising an increased gene expression in at least 150 genes, preferably at least the first 150 genes, in the list provided in Gene Set 11. In some embodiments, a “latent time 4” gene expression pattern has a gene expression pattern comprising an increased gene expression in 1-10, 11-20, 21-30, 31-40, 41-50, 51-60, 61-70, 71-80, 81-90, 91-100, 101-120, 121-140, 141-160, or 161-190 genes, preferably the first 1-10, first 11-20, first 21-30, first 31-40, first 41-50, first 51-60, first 61-70, first 71-80, first 81-90, first 91-100 genes, first 101-120, first 121-140, first 141-160, or first 161-190 genes in the list provided in Gene Set 11.

Overall, a phenotype and/or a gene expression pattern for a cancer sample or tumor cells can be used for applications such as admiration of a therapy based on detected phenotype or gene expression pattern, prediction of responsiveness to a therapy, and providing prognosis to a patient, as detailed below.

Detection Use and Techniques

a. Detection Use of the Tumor Phenotypes

Methods are provided for detecting a phenotype of a cancer sample, comprising detecting the presence of a CDH12-high phenotype, a CDH12-low phenotype, a KRT6A-high phenotype, a cycling-high phenotype, a UPK-high phenotype, or a KRT-high phenotype, in a cancer sample from the subject. Methods are also provided for detecting a phenotype having a gene expression pattern of latent time 0, time 1, time 2, time 3, or time 4 in a cancer sample. Detecting a phenotype includes measuring a corresponding gene expression (or mutation) pattern, wherein the corresponding signature set of genes, as well as its gene expression (or mutation) pattern, is detailed above.

In some embodiments, a method for detecting a phenotype of a cancer sample comprises detecting a CDH12-expressing tumor cell in the cancer sample, and detecting a CDH12-high phenotype in the CDH12-expressing tumor cell. In further embodiments, the CDH12-expressing tumor cell is a CDH12-positive epithelial cell.

In further embodiments, a method for detecting a phenotype of a cancer sample comprises detecting a ratio of the CDH12-high phenotype to any one of the other phenotypes (KRT6A-high/cycling-high/UPK-high/KRT-high). For example, a method detects the presence of the CDH12-high phenotype and detecting an absence of one or more of the KRT6A-high phenotype, the cycling-high phenotype, the UPK-high phenotype, and the KRT-high phenotype, wherein detecting the absence of a phenotype is detecting the presence of an expression pattern other than that for the phenotype. In another instance, a method detects a higher percentage of the presence of the CDH12-high phenotype than that of the presence of each one of the KRT6A-high phenotype, the cycling-high phenotype, the UPK-high phenotype, and the KRT-high phenotype. Yet in another embodiment, a method detects an absence of CDH12-high phenotype and the presence of one or more of the KRT6A-high phenotype, the cycling-high phenotype, the UPK-high phenotype, and the KRT-high phenotype.

In some embodiments, a method for detecting a phenotype of a cancer sample comprises detecting a CDH12-expressing tumor cell in the cancer sample, and detecting a CDH12-low phenotype in the CDH12-expressing tumor cell. In further embodiments, the CDH12-expressing tumor cell is a CDH12-positive epithelial cell.

In some embodiments, a method for detecting a phenotype of a cancer sample comprises detecting a KRT6A-expressing tumor cell in the cancer sample, and detecting a KRT6A-high phenotype in the KRT6A-expressing tumor cell. In further embodiments, the KRT6A-expressing tumor cell is a KRT6A-positive epithelial cell.

In some embodiments, a method for detecting a phenotype of a cancer sample comprises detecting a tumor cell expressing cell cycle-related genes in the cancer sample, and detecting a cycling phenotype in the cell cycle-related gene-expressing tumor cell. In further embodiments, the cell cycle-related gene-expressing tumor cell is an epithelial cell positive for one or more or all of KI67, SET and MYND domain containing 3 (SMYD3), centrosomal protein 192 (CEP192), AT-rich interaction domain 1B (ARID1B), Forkhead Box P1 (FOXP1), vascular endothelial growth factor A (VEGFA), and peroxisome proliferator-activated receptor gamma (PPARG).

In some embodiments, a method for detecting a phenotype of a cancer sample comprises detecting a UPK-expressing tumor cell in the cancer sample, and detecting a UPK-high phenotype in the UPK-expressing tumor cell. In further embodiments, the UPK-expressing tumor cell is a UPK-positive epithelial cell.

In some embodiments, a method for detecting a phenotype of a cancer sample comprises detecting a KRT13-expressing and/or KRT17-expressing tumor cell in the cancer sample, and detecting a KRT-high phenotype in the KRT13 and/or KRT 17-expressing tumor cell. In further embodiments, the KRT13 and/or KRT 17-expressing tumor cell is a KRT13⁺, KRT17⁺ epithelial cell.

In some embodiments, a method for detecting a gene expression pattern in a cancer sample comprises detecting a gene expression pattern of latent time 0 in the cancer sample. In some embodiments, a method for detecting a gene expression pattern in a cancer sample comprises detecting a gene expression pattern of latent time 1 in the cancer sample. In some embodiments, a method for detecting a gene expression pattern in a cancer sample comprises detecting a gene expression pattern of latent time 2 in the cancer sample. In some embodiments, a method for detecting a gene expression pattern in a cancer sample comprises detecting a gene expression pattern of latent time 3 in the cancer sample. In some embodiments, a method for detecting a gene expression pattern in a cancer sample comprises detecting a gene expression pattern of latent time 4 in the cancer sample.

In further embodiments, a method for detecting a gene expression pattern in a cancer sample comprises detecting a ratio of tumor cells having a gene expression pattern of latent time 0 and of latent time 1 versus tumor cells having a gene expression pattern of latent time 4 and of latent time 3. In another embodiment, a method for detecting a gene expression pattern in a cancer sample comprises detecting a ratio of tumor cells having a gene expression pattern of latent time 0 versus tumor cells having a gene expression pattern of latent time 4. In yet another embodiment, a method for detecting a gene expression pattern in a cancer sample comprises detecting both an expression pattern of latent time 0 and an expression pattern of latent time 1. In another embodiment, a method for detecting a gene expression pattern in a cancer sample comprises detecting both an expression pattern of latent time 4 and an expression pattern of latent time 3.

In some embodiments, a method for detecting a gene expression pattern in a biological sample from a cancer patient comprises detecting a gene expression pattern of latent time 0, 1, 2, 3, or 4 in a normal cell in the biological sample, and detecting a CDH12-high, a CDH12-low, a KRT6A-high, a cycling-high, a UPK-high, or a KRT-high phenotype in a tumor cell in the biological sample.

Various embodiments also provide for a method of detection of a CDH12+ tumor sample from a subject with bladder cancer, wherein the CDH12+ tumor sample is also positive for, or expresses, ALDH1A1, PD-L1, PD-L2, or a combination of the three, as well as ligand for CD49a, or wherein the CDH12+ tumor sample comprises CDH12+ tumor cells and CD49a+ T-cells. In some embodiments, the CDH12+ tumor sample is also detected with a gene expression pattern of the CDH12-high phenotype, or a gene expression of the CDH12-low phenotype.

Additional embodiments provide for a method of detecting a gene expression (or mutation) pattern in a CDH12-positive tumor sample, comprising assaying a tumor sample obtained from the subject, wherein the subject desires a determination regarding survival prognosis or treatment selection (responsiveness prognosis).

In some embodiments, assaying the tumor sample detects a higher gene expression in 1-50 genes in Gene Set 2-1, or detects a higher gene mutation in two or more of EIF4G3, ALAS1, NINL, NSD1, DFNA5, PABPC3, and TXNDC11. In some embodiments, assaying the tumor sample detects a higher gene expression in 51-100 genes in Gene Set 1, or detects a higher gene mutation in three or more of EIF4G3, ALAS1, NINL, NSD1, DFNA5, PABPC3, and TXNDC11. In some embodiments, assaying the tumor sample detects a higher gene expression in 100-200 genes in Gene Set 1, or detects a higher gene mutation in four or more of EIF4G3, ALAS1, NINL, NSD1, DFNA5, PABPC3, and TXNDC11. In some embodiments, assaying the tumor sample detects a higher gene expression in 200 or more genes in Gene Set 1, or detects a higher gene mutation in five or more of EIF4G3, ALAS1, NINL, NSD1, DFNA5, PABPC3, and TXNDC11. In some embodiments, assaying the tumor sample detects a higher gene expression in 30 or more genes in Gene Set 1, or detects a higher gene mutation in six or more of EIF4G3, ALAS1, NINL, NSD1, DFNA5, PABPC3, and TXNDC11.

In some embodiemnts, assaying the tumor sample detects a higher gene mutation in ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in two or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in three or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in four or more of ERBB2, FGFR3, PAPPA2, ASAP1, OCA2, NDC80, AP3D1, BAP1, KIFAP3, NOC3L, PAX7, and TN7RC18. In some embodiemnts, assaying the tumor sample detects a higher gene mutation in five or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in six or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in seven or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in eight or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in nine or more of ERBB2, FGFR3, PAPPA2, ASAP1, OCA2, NDC80, AP3D1, BAP1, KIFAP3, NOC3L, PAX7, and TN7RC18. In some embodiemnts, assaying the tumor sample detects a higher gene mutation in ten or more of ERBB2, FGFR3, PAPPA2, ASAP1, OCA2, NDC80, AP3D1, BAP1, KIFAP3, NOC3L, PAX7, and TNRC18. In some embodiemnts, assaying the tumor sample detects a higher gene mutation in 11 or more of ERBB2, FGFR3, PAPPA2, ASAP1, OCA2, NDC80, AP3D1, BAP1, KIFAP3, NOC3L, PAX7, and TNRC18.

In further embodiments, detecting a higher gene expression in one or more genes in Gene Set 1 is associated with detecting a higher gene mutation in one or more of EIF4G3, ALAS1, NINL, NSD1, DFNA5, PABPC3, and TXNDC11. In other embodiments, detecting an increased gene expression in one or more genes in Gene Set 2 (CDH12-low phenotype) is associated with detecting a higher gene mutation in ERBB2, FGFR3, PAPPA2, ASAP1, OCA2, NDC80, AP3D1, BAP1, KIFAP3, NOC3L, PAX7, and TNRC18. Further embodiments provide that this method of detection also includes detecting expression level of one or more of ALDH1A1, PD-L1, and PD-L2 in the tumor sample, and/or detecting presence of CD49+ CD8 T-cells in the tumor sample.

Further embodiments provide detecting any one or more of the genes listed in CDH12 subgroup, KRT13 subgroup, KRT6A subgroup, UPK subgroup, and Cycling subgroup of epithelial cells in a tumor sample from a subject. Provided in each subgroup is a list in descending order by “score.” Our scoring algorithm takes into account both the magnitude of the log FC as well as the FDR significance. So the genes decrease in both fold change and statistical significance as you go down the list. The comparison that was run to generate log FC in these lists was to compare gene expression in one subgroup versus all of the other subgroups combined. So the lists represent signatures that are positively associated with the respective subgroups.

In various embodiments, the one or more detected phenotypes listed above are used for a prognosis use, a selection of therapy, or a treatment use.

In various implementations, the genes listed in CDH12 subgroup, KRT13 subgroup, KRT6A subgroup, UPK subgroup, and Cycling subgroup of epithelial cells have an expression level above a reference, so as to indicate the presence or progression of the tumor. In some embodiments, a reference is from a pool of tumor samples including all of CDH12 subgroup, KRT13 subgroup, KRT6A subgroup, UPK subgroup, and Cycling subgroup.

A tumor sample may have a cellular composition including epithelial cells, fibroblasts, immune cells, and/or endothelial cells. In various embodiments wherein epithelial cells account for at least 90%, 85%, 80%, or 75% of cells in the tumor sample, detecting a CDH12-high, a CDH12-low, a KRT6A-high, a cycling-high, a UPK-high, or a KRT-high phenotype in the tumor cell may comprise detecting the CDH12-high, the CDH12-low, the KRT6A-high, the cycling-high, the UPK-high, or the KRT-high phenotype in at least 90%, 85%, 80%, or 75% of the cells in the tumor sample, or detecting the CDH12-high, the CDH12-low, the KRT6A-high, the cycling-high, the UPK-high, or the KRT-high phenotype in keratin-expressing cells in the tumor sample.

The cancer sample or the biological sample may be obtained from a patient with a cancer such as a bladder cancer. It may also be obtained from a patient with a MIBC. In another embodiment, the cancer sample or the biological sample is obtained from a patient with a urothelial carcinoma. In some implementations, the sample is obtained before a therapy or surgery is performed to the patient. In some implementations, the sample is obtained after a therapy or surgery is performed to the patient. In further implementations, a first sample is obtained before a therapy or surgery is performed to the patient, and a second sample is obtained after a therapy or surgery is performed to the patient.

b. Detection Techniques

In various aspects, measuring a gene expression pattern includes performing sequencing of mRNA, e.g., unbiased sequencing of single-nuclei mRNA. In other aspects, measuring a gene expression pattern includes contacting one or more detection agents that specifically bind to each of a combination of the genes and/or proteins, and quantifying levels of the one or more detection agents bound to each of the combination of the genes and/or proteins relative to a reference for each gene or protein.

In additional aspects, measuring a gene mutation pattern includes sequencing each target gene, e.g., unbiased sequencing of single-nucleoid DNA, and identifying a base substitution, deletion, and/or insertion in the sequenced target gene relative to a wild type of the target gene, and optionally further comparing the percentage of target genes having at least one of the base substitution, deletion, and insertion in the tumor cells relative to a reference level. In some embodiments, the reference mutation level is zero (i.e., wild type), and so the presence of a base substitution, deletion, and/or insertion identifies an “increased” mutation. In other embodiments, the reference mutation level is a percentage of base substitution, deletion, and/or insertion identified in a population of reference cells, and so a higher percentage of the base substitution, deletion, and/or insertion detected in target population of cells identifies an “increased” mutation. Wild type genes, their nomenclature, and their sequences are available in publicly accessible database such as GENBANK®, an NIH genetic sequence database. In various implementations, a detected gene sequence other than the wild type sequence in this database is considered a mutation.

### Reference Levels

A reference level (or amount), in some instances, is an average level in a whole cancer sample or whole biological sample (the whole cancer or biological sample having intra-sample heterogeneity), when the subject/test level is with respect to a subgroup, a phenotype, or a subpopulation. In other instances, a reference level is an average level in the rest of the whole cancer or biological sample, except for the subject/test level. In further instances, a reference level is the level in a non-cancerous sample or obtained from a subject without a cancer.

### Prognostic Uses

The one or more phenotypes of in tumor cells, and/or the one or more latent times of gene expression pattern of tumor cells, can be used for providing survival prognosis and/or therapeutic responsiveness prognosis to the subject.

In some embodiments, a method for providing prognosis for a subject with a cancer comprises detecting a CDH12-high phenotype in tumor cells (or keratin-expressing cells) of the subject, and providing a poorer survival prognosis or a poorer prognosis of responsiveness to a chemotherapy (e.g., neoadjuvant chemotherapy, or platinum-based neoadjuvant chemotherapy) or to merely a chemotherapy, for a subject treated or to be treated with (merely) the chemotherapy, relative to an immune checkpoint inhibitor therapy.

In some embodiments, a method for providing prognosis for a subject with a cancer comprises detecting a greater occurrence/percentage of a CDH12-high phenotype than any one of a KRT6A-high, a cycling-high, a KRT-high, and a UPK-high phenotype in tumor cells of the subject, (or a presence of CDH12-high phenotype and an absence of KRT6A-high, a cycling-high, a KRT-high, and a UPK-high phenotype), and providing a poorer survival prognosis or a poorer prognosis of responsiveness to a chemotherapy (e.g., neoadjuvant chemotherapy, or platinum-based neoadjuvant chemotherapy) or to merely a chemotherapy, for a subject treated or to be treated with (merely) the chemotherapy, relative to an immune checkpoint inhibitor therapy.

In another embodiment, a method for providing prognosis for a subject with a cancer comprises providing a poorer survival prognosis or a poorer prognosis of responsiveness to a chemotherapy (e.g., neoadjuvant, or platinum-based neoadjuvant chemotherapy) or surgery or to a treatment consisting of just a chemotherapy (without an immune checkpoint inhibitor), relative to an immune checkpoint inhibitor therapy, for a subject detected with a CDH12-high phenotype, or detected with a greater occurrence/percentage of CDH12-high than other phenotypes, in tumor cells (or keratin-expressing cells) of the subject.

In some embodiments, a method for providing prognosis for a subject with a cancer comprises detecting a CDH12-high phenotype in tumor cells (or keratin-expressing cells) of the subject, and providing a better survival prognosis or a better prognosis of responsiveness to an immune checkpoint inhibitor, for a subject treated or to be treated with the immune checkpoint inhibitor, relative to treatment with a chemotherapy (e.g., platinum-based neoadjuvant chemotherapy), the surgery or no treatment.

In another embodiment, a method for providing prognosis for a subject with a cancer comprises providing a better survival prognosis or a better prognosis of responsiveness to an immune checkpoint inhibitor or an immunotherapy, relative to treatment with a chemotherapy (e.g., platinum-based neoadjuvant chemotherapy), the surgery or no treatment, for a subject detected with a CDH12-high phenotype in tumor cells (or keratin-expressing cells) of the subject.

In some embodiments, a method for providing prognosis for a subject with a cancer comprises providing a better survival prognosis or a better prognosis of responsiveness to an immune checkpoint inhibitor or an immunotherapy, relative to treatment with a chemotherapy (e.g., platinum-based neoadjuvant chemotherapy) or no treatment, for a subject detected in a cancer sample with a greater occurrence/percentage of a CDH12-high phenotype over another phenotype (e.g., KRT6A-high, cycling, UPK, and KRT).

In some embodiments, a method for providing prognosis for a subject with a cancer comprises detecting a phenotype having a gene expression pattern of latent time 0 or latent time 1 in normal cells within a biopsy sample from a subject with a cancer, and providing a better survival prognosis or a better prognosis of responsiveness to an immune checkpoint inhibitor, for the subject treated or to be treated with the immune checkpoint inhibitor, relative to treatment with a chemotherapy (e.g., the platinum-based neoadjuvant chemotherapy) or no treatment.

In another embodiment, a method for providing prognosis for a subject with a cancer comprises providing a better survival prognosis or a better prognosis of responsiveness to an immune checkpoint inhibitor, relative to treatment with a chemotherapy (e.g., the platinum-based neoadjuvant chemotherapy) or no treatment, for a subject detected with a phenotype having a gene expression pattern of latent time 0 or latent time 1 in normal cells within a biopsy sample from a subject with a cancer.

In some embodiments, a method for providing prognosis for a subject with a cancer comprises detecting a phenotype having a gene expression pattern of latent time 4 or latent time 3 in normal cells within a biopsy sample from a subject with a cancer, and providing a poorer survival prognosis or a poorer prognosis of responsiveness to the immune checkpoint inhibitor, for the subject treated or to be treated with the immune checkpoint inhibitor, relative to treatment with a chemotherapy (e.g, platinum-based chemotherapy) or no treatment.

In another embodiment, a method for providing prognosis for a subject with a cancer comprises providing a poorer survival prognosis or a poorer prognosis of responsiveness to the immune checkpoint inhibitor, relative to treatment with a chemotherapy (e.g., platinum-based chemotherapy) or no treatment, for a subject detected with a phenotype having a gene expression pattern of latent time 4 or latent time 3 in normal cells within a biopsy sample from a subject with a cancer.

In some embodiments, a method for providing prognosis for a subject with a cancer comprises detecting a KRT-high phenotype in tumor cells (or KRT13-expressing cells) of the subject, and providing a better survival prognosis or a better prognosis of responsiveness to a chemotherapy (e.g., neoadjuvant chemotherapy, or platinum-based neoadjuvant chemotherapy), for a subject treated or to be treated with (merely) the chemotherapy, relative to no treatment. The KRT-phenotype in a tumor sample indicates that the tumor is chemo-sensitive.

In some embodiments, a method for providing prognosis for a subject with a cancer comprises detecting a UPK-high phenotype in tumor cells (or UPK-expressing cells) of the subject, and providing a better survival prognosis or a better prognosis of responsiveness to a chemotherapy (e.g., neoadjuvant chemotherapy, or platinum-based neoadjuvant chemotherapy), for a subject treated or to be treated with (merely) the chemotherapy, relative to no treatment. The UPK-phenotype in a tumor sample indicates that the tumor is chemo-sensitive.

Further embodiments provide methods for use of the CDH12+ tumor sample to provide prognosis for a subject in need thereof. In some embodiments, a tumor sample with CDH12 expression level below a reference value has a good prognosis, e.g., above median survival/responsiveness prognosis, with a cisplatin-based neoadjuvant chemotherapy and/or surgery. In some embodiments, a tumor sample with CDH12 expression level above a reference value has a poor prognosis, e.g., below median survival/responsiveness prognosis, with a cisplatin-based neoadjuvant chemotherapy and/or surgery. In some embodiments, a tumor sample with CDH12 expression level above a reference value has a good prognosis, e.g., above median survival/responsiveness prognosis, with an immune checkpoint therapy (e.g., immune checkpoint inhibitor).

### Treatment Methods

A method of treating, reducing the severity, and/or reducing the progression of a cancer in a subject may include administering a neoadjuvant chemotherapy and/or performing surgery or radiation to the subject who has been determined to have an expression level of CDH12 from a cancerous tissue of the subject below a reference value, or administering an immune checkpoint inhibitor to the subject who has been determined to have an expression level of CDH12 from the cancerous tissue of the subject above a reference value.

In various embodiments, methods for treating, reducing the severity, and/or reducing the progression of a cancer in a subject, comprise administering a therapeutically effective amount of an immune checkpoint inhibitor, a TGFβ inhibitor, an anti-angiogenic therapy, or a combination thereof to the subject, wherein the subject has been determined to have a CDH12-high phenotype or CDH12-high gene mutation pattern in a cancer sample obtained from the subject.

Preferably, a method for treating a subject determined with a CDH12-high phenotype (including CDH12-high gene mutation pattern), a greater occurrence/percentage of CDH12-high than other phenotypes, and/or a gene expression pattern of latent time 0 or latent time 1 in the subject's cancer sample includes administering a therapeutically effective amount of an immune checkpoint inhibitor or a combination of the immune checkpoint inhibitor and a chemotherapy, rather than administering merely a chemotherapy. Alternatively, a method for treating a subject determined with a CDH12-high phenotype or CDH12-high gene mutation pattern in the subject's cancer sample includes administering a therapeutically effective amount of a TGFβ inhibitor, an anti-angiogenic therapy, or a combination thereof to the subject.

In other embodiments, methods for treating, reducing the severity, and/or reducing the progression of a cancer in a subject, comprise administering a therapeutically effective amount of a chemotherapy to the subject, wherein the subject has been determined to have a CDH12-low phenotype including CDH12-low gene mutation pattern and/or a gene expression pattern of latent time 4 or latent time 3 in the cancer sample from the subject.

In yet another embodiment, methods for treating, reducing the severity, and/or reducing the progression of a cancer in a subject, comprise administering a therapeutically effective amount of a chemotherapy to the subject, wherein the subject has been determined to have an absence of CDH12-high phenotype with the presence of one or more of the KRT6A-high phenotype, the cycling-high phenotype, the UPK-high phenotype, and the KRT-high phenotype, and/or determined to have a gene expression pattern of latent time 4 or latent time 3 in the cancer sample from the subject.

Additionally, after the administration of the neoadjuvant chemotherapy, if residue disease of the cancer (e.g., residue or relapsed cancerous tissue) is identified, a method for treating, reducing the severity, and/or reducing the progression of the cancer comprises administering to the subject a therapeutically effective amount of an anti-PDL1 or anti-PD1 therapy (e.g., monoclonal antibody), an anti-CTLA4 therapy (e.g., monoclonal antibody), or a combination thereof, wherein the subject is detected with a CDH12-high phenotype or CDH12-high gene mutation pattern in a cancer sample from the subject.

Alternatively, after the administration of the neoadjuvant chemotherapy, if residue disease of the cancer (e.g., residue or relapsed cancerous tissue) is identified, a method for treating, reducing the severity, and/or reducing the progression of the cancer comprises administering to the subject a therapeutically effective amount of an anti-TIM3 therapy (e.g., monoclonal antibody), an anti-TIGIT therapy (e.g., monoclonal antibody), or a combination thereof, wherein the subject is detected with a CDH12-low phenotype or CDH12-low gene mutation pattern in a cancer sample from the subject.

Further embodiments provide a method for treating, reducing the severity, and/or slowing the progression of a cancer in a subject comprises performing a treatment based on a good survival prognosis or responsiveness prognosis noted above.

Additional embodiments provide methods for detecting a phenotype or gene mutation expression pattern of a cancer in a subject and treating, reducing the severity of and/or slowing the progression of the cancer in the subject, which include detecting a CDH12-high phenotype of a cancer sample obtained from the subject, and administering a therapeutically effective amount of an immune checkpoint inhibitor, a combination of the immune checkpoint inhibitor and a neoadjuvant chemotherapy, a transforming growth factor beta (TGFβ) inhibitor, and/or an anti-angiogenic therapy, to the subject, thereby treating, reducing the severity of and/or slowing the progression of the cancer.

Examples of immune checkpoint inhibitors, or immune checkpoint blockade (ICB) therapeutics, include but are not limited to, an anti-PD-L1 antibody, an antibody against PD-1, an antibody against PD-L2, an antibody against CTLA-4, an antibody against KIR, an antibody against IDO1, an antibody against ID02, an antibody against TIM-3, an antibody against LAG-3, an antibody against OX40R, and an antibody against PS.

Other examples of immune checkpoint inhibitors include inhibitors of leukocyte surface antigen CD47 (antigenic surface determinant protein OA3 or integrin associated protein or protein MER6 or CD47), and such examples are magrolimab (by Forty Seven), IBI-188 (by Innovent Biologics), ALX-148 (by ALX Oncology), AO-176 (by Arch Oncology), and CC-90002 (by Bristol-Myers Squibb).

Another class of exemplary immune checkpoint inhibitors or immune checkpoint blockade therapeutics include antagonists or inhibitors of T cell immunoreceptor with Ig and ITIM domains (V set and immunoglobulin domain containing protein 9 or V set and transmembrane domain containing protein 3 or TIGIT), and such examples are tiragolumab (by Genentech), AB-154 (by Arcus Biosciences), BMS-986207 (by Bristol-Myers Squibb), vibostolimab (by Merck), and BGBA-1217 (by BeiGene).

Yet another class of exemplary immune checkpoint inhibitors or immune checkpoint blockade therapeutics include antagonists of adenosine receptor A2a (ADORA2A) or A2b (ADORA2B), and examples include AB-928 (by Arcus Biosciences), ciforadenant (by Corvus Pharmaceuticals), HTL-1071 (by AstraZeneca), PBF-509 (by Novartis), and EOS-100850 (by iTeos Therapeutics).

In one embodiment, the immune checkpoint inhibitor is humanized monoclonal anti-programmed death ligand 1 (PD-L1) antibody, atezolizumab. In another embodiment, the immune checkpoint inhibitor is an anti-PD-L1 antibody/inhibitor such as avelumab, cemiplimab, durvalumab, KN035, CK-301, AUNP12, CA-170, MPDL3280A(RG7446), MEDI4736 and BMS-936559.

In another embodiment, the immune checkpoint inhibitor is an anti-PD-1 antibody such as pembrolizumab (formerly lambrolizumab or MK-3475), nivolumab (BMS-936558), cemiplimab, spartalizumab, camrelizumab, sintilimab, tislelizumab, toripalimab, Pidilizumab (CT-011), AMP-224, or AMP-514.

Further examples of immune checkpoint inhibitor, or immune checkpoint blockade (ICB) therapeutics, include but are not limited to, B7-DC-Fc fusion proteins such as AMP-224, anti-CTLA-4 antibodies such as tremelimumab (CP-675,206) and ipilimumab (MDX-010), antibodies against the B7/CD28 receptor superfamily, anti-Indoleamine (2,3)-dioxygenase (IDO) antibodies, anti-IDO1 antibodies, anti-ID02 antibodies, tryptophan, tryptophan mimetic, 1-methyl tryptophan (1-MT)), Indoximod (D-1-methyl tryptophan (D-1-MT)), L-1-methyl tryptophan (L-1-MT), TX-2274, hydroxyamidine inhibitors such as INCB024360, anti-TIM-3 antibodies, anti-LAG-3 antibodies such as BMS-986016, recombinant soluble LAG-3Ig fusion proteins that agonize MHC class II-driven dendritic cell activation such as IMP321, anti-KIR2DL1/2/3 or anti-KIR) antibodies such lirilumab(IPH2102), urelumab (BMS-663513), anti-phosphatidylserine (anti-PS) antibodies such as Bavituximab, anti-idiotype murine monoclonal antibodies against the human monoclonal antibody for N-glycolil-GM3 ganglioside such as Racotumomab (formerly known as 1E10), anti-OX40R antibodies such as IgG CD134 mAb, anti-B7-H3 antibodies such as MGA271, and small interfering (si) RNA-based cancer vaccines designed to treat cancer by silencing immune checkpoint genes.

Neoadjuvant chemotherapy may be a type of cancer treatment where chemotherapy drugs are administered before surgical extraction of the tumor or another main treatment, usually with the goal of shrinking a tumor or stopping the spread of cancer to make surgery less invasive and more effective. Conversely, adjuvant chemotherapy is administered after surgery to kill any remaining cancer cells with the goal of reducing the chances of recurrence. Examples of neoadjuvant therapy include chemotherapy, radiation therapy, and hormone therapy. Exemplary chemotherapeutics include but art not limited alkylating agents (e.g., Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin), mitrosoureas (e.g., carmustine, lomustine, streptozocin), antimetabolites (Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridine/tipiracil combination), anti-tumor antibiotics, topoisomerase inhibitor, mitotic inhibitors, corticosteroids.

Platinum-based chemotherapeutics include cisplatin, carboplatin, oxaliplatin, nedaplatin, and lobaplatin.

Cisplatin-based neoadjuvant combination chemotherapy comprises one or more cisplatin-based chemotherapeutic agent and one or more adjuvants. Generally, neoadjuvant therapy is the administration of therapeutic agents before a main treatment; and in some cancer patients the main treatment is cystectomy, or interval debulking surgery. In some embodiments, neoadjuvant chemotherapy is chemotherapy given prior to the surgical procedure. In other embodiments, adjuvant chemotherapy is given to prevent a possible cancer recurrence. Exemplary cisplatin-based neoadjuvants (or adjuvants) include, but are not limited to, (1) methotrexate, vinblastine, doxorubicin, and cisplatin (MVAC); (2) dose-dense, or accelerated, MVAC (ddMVAC); (3) gemcitabine and cisplatin (GC); (4) paclitaxel/gemcitabine/cisplatin (PGC); (5) cisplatin/methotrexate/vinblastine (CMV); (6) a combination thereof, such as ddMVAC/GC/MVAC. Usually the cisplatin-based neoadjuvants (or adjuvants) are given for more than one cycle, e.g., for at least 3 cycles, for at least 4 cycles, for at least 5 cycles.

Exemplary TGFβ inhibitors can be an antibody, an antisense oligodeoxynucleotide, an adoptive T cell, a small molecule, include but art not limited to Fresolimumab, LY3022859, PF-03446962, SAR439459, AVID200, Bintrafusp alfa, Trabedersen, and Galunisertib.

Exemplary anti-angiogenic therapies include but are not limited to Axitinib (INLYTA®), Bevacizumab (AVASTIN®), Cabozantinib (COMETRIQ®), Everolimus (AFINITOR®), Lenalidomide (REVLIMID®), Lenvatinib mesylate (LENVIMA®), Pazopanib (VOTRIENT®), Ramucirumab (CYRAMZA®), Regorafenib (STIVARGA®), Sorafenib (NEXAVAR®), Sunitinib (SUTENT®), Thalidomide (THALOMID®), Vandetanib (CAPRELSA®), and Ziv-aflibercept (ZALTRAP®).

Exemplary anti-CTLA4 therapies include but are not limited to Ipilimumab and tremelimumab.

Exemplary anti-TIGIT therapies include but are not limited to Tiragolumab and BMS-986207.

Exemplary anti-TIM3 therapies include but are not limited to Cobolimab, LY3321367, Sym023, and BMS-986258.

In various implementations, one of more therapeutics described herein is formulated or provided in a pharmaceutical composition, comprising the therapeutics and a pharmaceutically acceptable excipient or carrier. Pharmaceutical compositions according to the invention may be formulated for delivery via any route of administration. Two or more methods of administration may be used at the same time under certain circumstances. For example, chemotherapy drugs may be administered orally (oral chemotherapy), or injected into a muscle (intramuscular injection), injected under the skin (subcutaneous injection), or into a vein (intravenous chemotherapy). In special cases, chemotherapy drugs may be injected into the fluid around the spine (intrathecal chemotherapy).

Additional implementations provide that one or more therapeutics described herein is formulated for administration at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m², or a combination thereof. In some embodiments, the one or more therapeutics is formulated for administration about 1-3 times per day, 1-7 times per week, 1-9 times per month, or 1-12 times per year. In some embodiments, the one or more therapeutics is formulated for administration for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years.

Additional embodiments provide that a subject's gene expression levels of one or more genes in the list provided in Gene Set 2 in CDH12+ tumor cells are below a reference value prior to receiving a chemotherapy (e.g., a cisplatin-based chemotherapy), which rise to above a reference value after receiving the chemotherapy, and this subject will likely respond to an immune checkpoint inhibitor (e.g., an anti-PD-L1 antibody such as atezolizumab), so the subject is selected to receive an immune checkpoint inhibitor in addition to or in place of chemotherapy. In some embodiments, the subject for a method of treating, reducing severity, or slowing progressin of a cancer is resistant to or unresponsive of chemotherapeutic agents, and the subject is detected with a CDH12-high phenotype in tumor cells of the subject.

Various embodiments of the present invention provide for a method of treating a cancer subject, comprising one or more of: administering a neoadjuvant chemotherapy before surgery or radiation, performing the surgery or the radiation, and administering an adjuvant therapy to a subject in need thereof, wherein the subject has been determined with a CDH12-low phenotype or a gene expression pattern of latent time 4 or latent time 3 in the cancer.

Various embodiments of the present invention provide for a method of treating a cancer subject, comprising: obtaining result of an analysis of expression levels in a tumor sample of a subject of one or more genes in the list provided in Gene Set 1 (e.g., in CDH12-expressing tumor cells), and administering an immune checkpoint inhibitor to the subject when the expression levels of the one or more genes are above a reference value.

Various embodiments of the present invention provide for a method of treating a cancer subject, comprising: obtaining result of an analysis of expression levels in a tumor sample of a subject of one or more genes in the list provided in Gene Set 1 (e.g., in CDH12-expressing tumor cells), and administering a neoadjuvant chemotherapy in combination with a primary treatment such as surgery or radiation to the subject when the expression levels of the one or more genes are below a reference value.

Various embodiments of the present invention provide for a method of treating a cancer subject, comprising: requesting result of an analysis of expression levels of one or more genes in the list provided in Gene Set 1 in a tumor sample (e.g., CDH12-expressing epithelial cell subpopulation of tumor sample) of a subject, and administering an immune checkpoint inhibitor to the subject when the expression levels of the one or more genes are above a reference value.

Various embodiments of the present invention provide for a method of treating a cancer subject, comprising: requesting result of an analysis of expression levels of one or more genes in the list provided in Gene Set 1 in a tumor sample (e.g., CDH12-expressing epithelial cell subpopulation tumor sample) of a subject, and administering a neoadjuvant chemotherapy in combination with a primary treatment such as surgery or radiation to the subject when the expression levels of the one or more genes are below a reference

Various embodiments provide for a method of selecting a cancer patient for administration of an immune checkpoint inhibitor, comprising detecting a CDH12-high phenotype and/or a gene expression pattern of latent time 0 or latent time 1 in a sample of tumor cells from the patient, and selecting the patient for receiving the immune checkpoint inhibitor.

Various embodiments provide for a method of selecting a cancer patient for administration of a chemotherapy, comprising detecting a CDH12-low phenotype and/or a gene expression pattern of latent time 4 or latent time 3 in a sample of tumor cells from the patient, and selecting the patient for receiving the chemotherapy.

### Kits/Systems

Kits for detecting an expression pattern in a biological sample, classifying a cancer in a subject, and/or providing prognosis for the subject, are also provided. In some embodiments, the kits include (i) one or more detection agents that specifically bind to each of a combination of genes and/or proteins encoded by those in Gene Set 1, one or more detection agents that specifically bind to each of a combination of genes and/or proteins encoded by those in Gene Set 3, one or more detection agents that specifically bind to each of a combination of genes and/or proteins encoded by those in Gene Set 4, one or more detection agents that specifically bind to each of a combination of genes and/or proteins encoded by those in Gene Set 5, and/or one or more detection agents that specifically bind to each of a combination of genes and/or proteins encoded by those in Gene Set 6; and (ii) instructions for using the one or more detection agents to detect the expression pattern in the biological sample, classify the cancer in the subject, and/or provide prognosis for the subject.

Further embodiments of the kits additionally include (iii) one or more detection agents that specifically bind to each of a combination of genes and/or proteins encoded by those in Gene Set 7, one or more detection agents that specifically bind to each of a combination of genes and/or proteins encoded by those in Gene Set 8, one or more detection agents that specifically bind to each of a combination of genes and/or proteins encoded by those in Gene Set 9, one or more detection agents that specifically bind to each of a combination of genes and/or proteins encoded by those in Gene Set 10, and/or one or more detection agents that specifically bind to each of a combination of genes and/or proteins encoded by those in Gene Set 11.

In some embodiments, the one or more detection agents are oligonucleotide probes, nucleic acids, DNAs, RNAs, peptides, proteins, antibodies, aptamers, or small molecules, or a combination thereof.

In some embodiments, the detection is performed by single-nuclei sequencing. In some embodiments the detection is performed using a microarray. The microarray can be an oligonucleotide microarray, DNA microarray, cDNA microarrays, RNA microarray, peptide microarray, protein microarray, or antibody microarray, or a combination thereof.

Systems are also provided for treating, reducing the likelihood of having, reducing the severity of, and/or slowing the progression of a cancer in a subject. In some embodiments, the systems include (i) one or more detection agents that specifically bind to each of a combination of genes and/or proteins encoded by those in Gene Set 1; and (ii) a quantity of a therapeutic; and optionally (iii) instructions for using the one or more detection agents and the therapeutic to treat, reduce the likelihood of having, reduce the severity of, and/or slow the progression of the cancer in the subject. In further embodiments, one or more therapeutics are included in the systems, such as an immune checkpoint inhibitor, a chemotherapeutic, an anti-angiogenic agent, an anti-TIGIT agent, an anti-TIM3 agent, and/or a TGFβ inhibitor.

In some embodiments, a system for treating a subject having a cancer with a CDH12-high expression pattern includes: (i) a quantity of a therapeutic comprising an immune checkpoint inhibitor, a TGFβ inhibitor, an anti-angiogenic therapy, or a combination thereof; and (ii) one or more detection agents that specifically bind to each of a combination of genes and/or proteins encoded by those in Gene Set 1; and optionally (iii) instructions for using the therapeutic and the one or more detection agents to treat the subject having the cancer with the CDH12-high expression pattern.

Use of Identified Signature Genes with Machine Learning Algorithm

Each of the gene set provided in Gene Sets 1-11 represent a signature set for the indicated phenotype. Additional embodiments provide a process including: detecting the presence or absence of a combination of signature sets (e.g., for 2, 3, 4, or more phenotypes), wherein the combination is identified through a machine learning algorithm such as a Naïve Baees Classifier, K-means Clustering, Support Vector Machine, Linear Regression, Logistic Regression, Artificial Neural Network, Decision Trees, Random Forrests, Nearest Neighbours algorithm, or any other algorithm, for combining genes from the signature sets, so as to classify patients or predict their response to a given therapy.

Some embodiments provide a gene selection method, wherein the method includes detecting expression levels for a combination of genes in each of a plurality of biological samples, wherein the combination of genes comprises those listed in two or more of Gene Sets 2-6, and wherein the plurality of biological samples are obtained from patients receiving a cancer therapy; and identifying genes from the combination based on their detected expression levels or relative expression levels via a machine learning algorithm to correlate with each patient's response to the cancer therapy, thereby selecting a set of genes associated with responsiveness to the cancer therapy.

Additional embodiments of the invention include:

01). A method for treating a subject with cancer, comprising:
administering a neoadjuvant chemotherapy before surgery or radiation, performing the surgery or the radiation, and/or administering an adjuvant therapy following the surgery or the radiation, to a subject detected with an expression level of cadherin 12 (CDH12) below a reference value in a tumor sample of the subject.
   02). A method for treating a subject with cancer, comprising:
administering an immune checkpoint inhibitor to a subject detected with an expression level of cadherin 12 (CDH12) above a reference value in a tumor sample of the subject.
   03). The method in paragraph 02, wherein the tumor sample is obtained from the subject who has received a neoadjuvant chemotherapy.
   04). The method in paragraph 03, wherein before receiving the neoadjuvant chemotherapy, the subject's expression level of CDH12 in the tumor sample is below the reference value.
   05). The method in paragraph 02, wherein the tumor sample is obtained from the subject who has not received a neoadjuvant chemotherapy.
   06). The method in any of paragraphs 02-05, further comprising one or more of administering a platinum-based neoadjuvant chemotherapy before surgery or radiation, performing the surgery or the radiation, and administering an adjuvant therapy following the surgery or the radiation, to the subject detected with the expression level of CDH12 above the reference value.
   07). The method in paragraph 02, wherein the tumor sample is further detected with expression of aldehyde dehydrogenase 1 family member A1 (ALDH1A1), programmed death-ligand 1 (PD-L1), programmed cell death ligand 2 (PD-L2), or a combination thereof, and/or detected with CD49+CD8+ T-cells in the tumor sample.
   08). A method for treating a subject with cancer, comprising:
measuring an expression level of cadherin 12 (CDH12) in a tumor sample of the subject; and
performing one or more of administering a neoadjuvant chemotherapy before surgery or radiation, performing the surgery or the radiation, and administering an adjuvant therapy following the surgery or the radiation, to the subject if the expression level of the CDH12 in the tumor sample is below a reference value, or administering an immune checkpoint inhibitor to the subject if the expression level of the CDH12 in the tumor sample is above a reference value.
   09). A method for detecting cadherin (CDH) level in a subject in need thereof, comprising: measuring expression level of CDH12 in a tumor sample of the subject, wherein the subject has cancer and the tumor sample comprises cancerous tissue or cells.
   10). The method in paragraph 09, wherein measurement comprises single nuclei RNA sequencing of the tumor sample.
   11). The method in any one of paragraphs 01-10, wherein the subject is a human and the cancer comprises bladder cancer.
   12). The method in any one of paragraphs 01-10, wherein the subject has muscle invasive bladder cancer (MIBC), and the tumor sample comprises urothelial carcinoma tissue.
   13). The method in paragraph 02, wherein the subject has undergone cystectomy, interval debulking surgery, or both.
   14). The method in any one of paragraphs 01, 08, and 09, wherein the subject has not received neoadjuvant chemotherapy.
   15). The method in any one of paragraphs 01, 08, and 09, wherein the measurement comprises measuring a first expression level of CDH12 before a neoadjuvant chemotherapy is administered to the subject, and measuring a second expression level of CDH12 after the neoadjuvant chemotherapy is administered to the subject.
   16). The method in any one of paragraphs 01-08, wherein the reference value is expression level of CDH12 from a non-cancerous tissue of a subject or from a subject free or cured of the cancer.
   17). The method in any one of paragraphs 01, 03-06, and 08, wherein the neoadjuvant chemotherapy comprises one or more of (1) methotrexate, vinblastine, doxorubicin, and cisplatin (MVAC), (2) dose-dense, or accelerated, MVAC (ddMVAC), (3) gemcitabine and cisplatin (GC), (4) paclitaxel, gemcitabine, and cisplatin (PGC), and (5) cisplatin, methotrexate, and vinblastine (CMV).
   18). The method in any one of paragraphs 02-08, wherein the immune checkpoint inhibitor comprises one or more of an anti-PD-L 1 antibody, an anti-PD-1 antibody, an anti-PD-L2 antibody, an anti-CTLA-4 antibody, an anti-IDO1 antibody, an anti-ID02 antibody, an anti-TIM-3 antibody, an anti-LAG-3 antibody, an anti-OX40R antibody, and an anti-PS antibody.
   19). A method of providing prognosis for a human subject suffering from or diagnosed with muscle invasive bladder cancer, comprising:
measuring expression level of CDH12 in a urothelial tissue sample of the subject,
wherein the subject is indicated as likely to respond to a neoadjuvant chemotherapy and/or a cystectomy when the expression level of CDH12 is below a reference value, and wherein the subject is indicated as unlikely to respond to the neoadjuvant chemotherapy or the cystectomy when the expression level of CDH12 is above a reference value, thereby providing a prognosis for the subject.
   20). A method of providing prognosis for a human subject suffering from or diagnosed with muscle invasive bladder cancer, comprising:
detecting expression level of CDH12 in a urothelial tissue sample of the subject above a reference value, wherein the subject is indicated as likely to respond to an immune checkpoint inhibitor, thereby providing a prognosis for the subject.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1). A CDH12+ Epithelial Cell Subpopulation in Bladder Tumors Responds Diametrically to Chemotherapy and Immunotherapy

Identification of Stem-Like CDH12-Expressing Epithelial Cells.

We performed the first comprehensive profiling of high-grade urothelial MIBCs using single-nucleus RNA-sequencing (snSeq) on 25 treatment-naïve patients, with surgery (TURBT/cystectomy) as their only treatment. Our study characterizes intratumoral heterogeneity and deconvolutes current molecular subtypes into their normal constituent parts. The higher resolution of snSeq provided new insights for developing more effective prognostic and predictive tools.

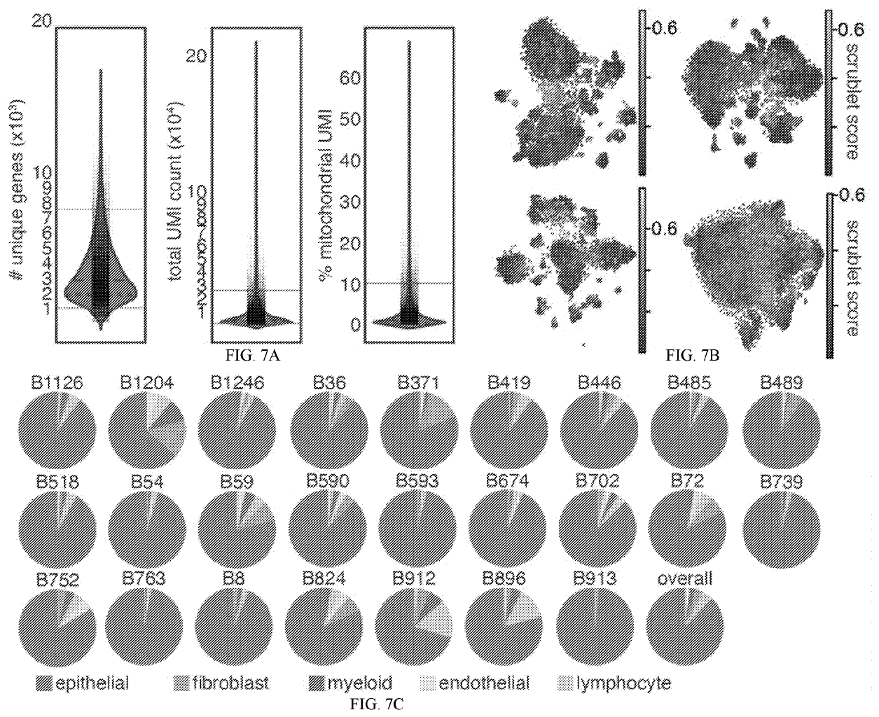

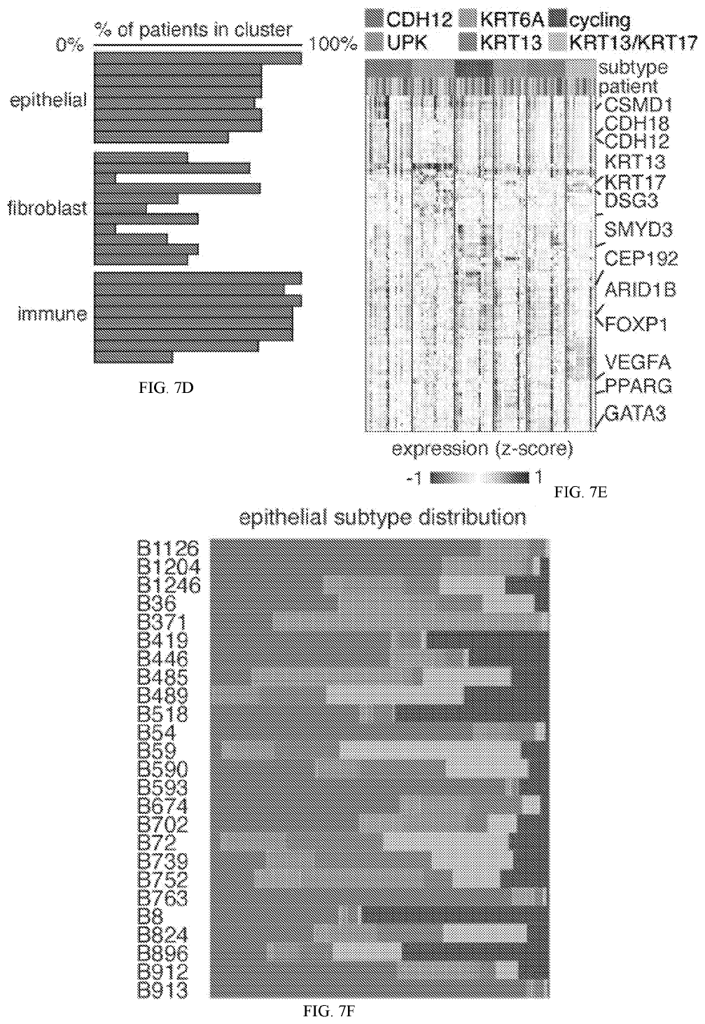

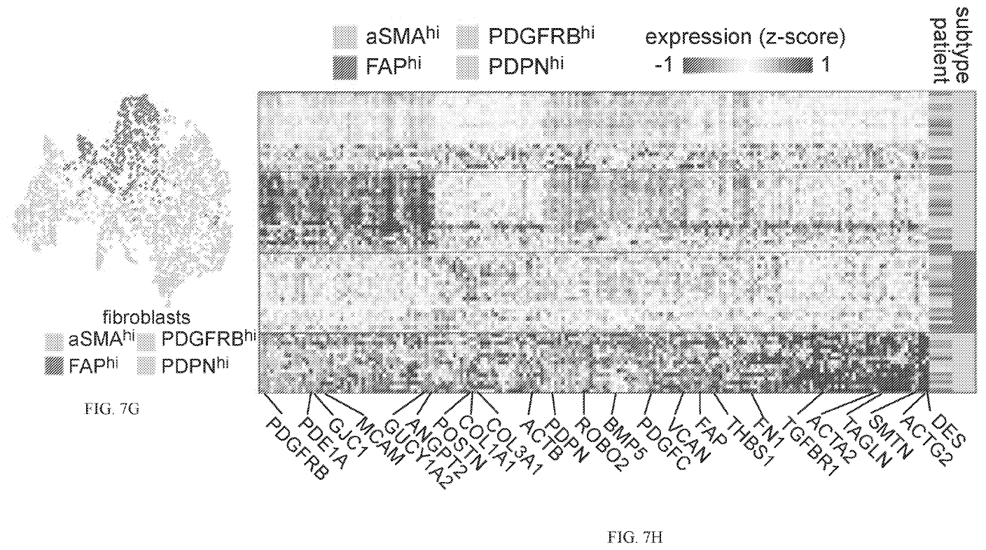

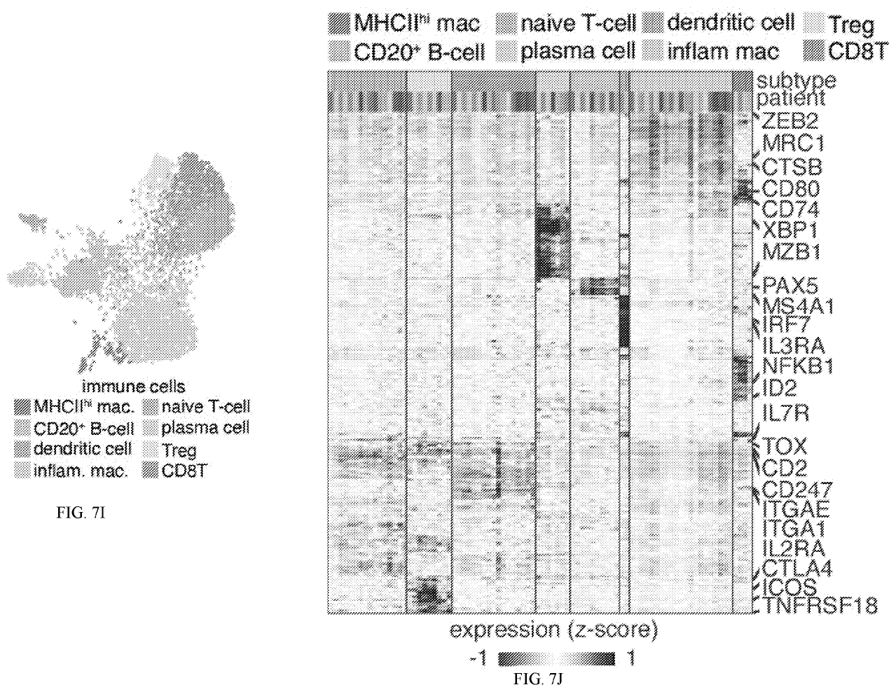

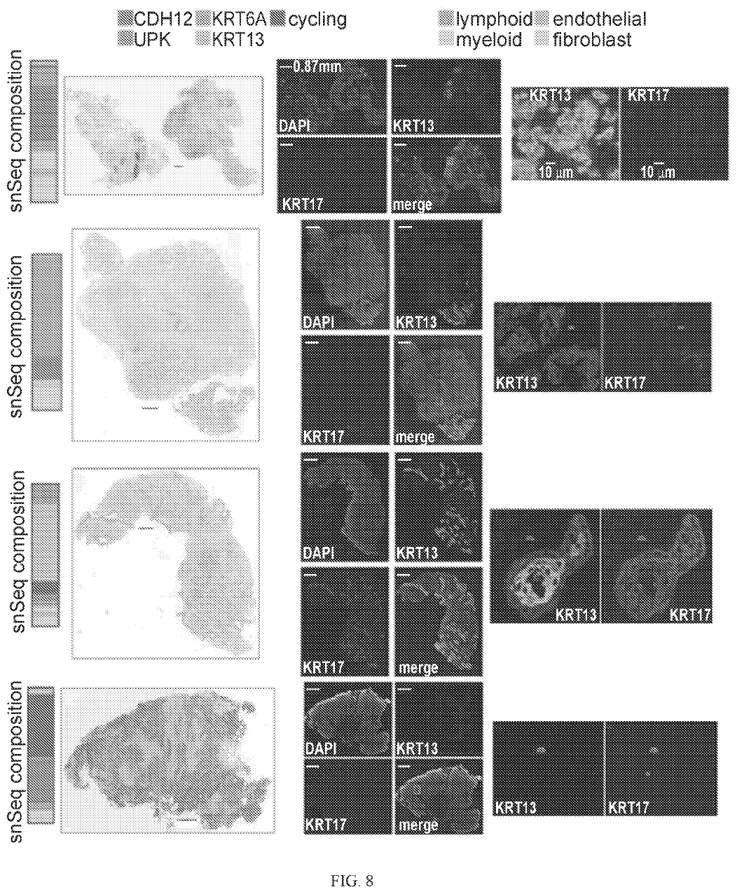

Toward the goal of characterizing intratumoral heterogeneity, we first looked at the overall cellular composition of the profiled MIBC tumors based on snSeq cell type proportions (FIG. 1A, 7A, 7B, and Table 1). The tumors were composed of about 90% epithelial cells, about 5% immune cells (including lymphocyte and myeloid), about 3% fibroblasts, and about 2% endothelial cells as annotated based on their corresponding expression of keratins (as a marker of epithelial cells), protein tyrosine phosphatase receptor type C (PTPRC; as a marker of immune cells), collagens (as marker of fibroblasts), and platelet/endothelial cell adhesion marker-1 (PECAMI) and von Willebrand factor (VWF) (both as markers of endothelial cells), respectively, among other key marker genes (FIGS. 1B, 1C and FIG. 7C). Unsupervised clustering of the epithelial compartment alone identified clusters with differential expression of KRT3 and KRT17—which were combined into one cluster (KRT13)—and uroplakins (UPK), KRT6A, cell-cycle-related genes (cycling), as well as a distinct cellular population expressing CDH12 along with other epithelial markers (FIG. 1D and FIG. 7E). We observed substantial inter-tumoral heterogeneity in epithelial compositions (FIG. 7F). The aforementioned genes were used to annotate the clusters because their high expression denoted unique clusters and the genes hold functional relevance (a listing of differentially expressed genes in descending order by scores of log FC>0 and FDR<0.05 for each cluster is included in the priority application, U.S. 63/197,129 filed Jun. 4, 2021, content of which has been incorporated by reference; and a refined listing by log FC>1.2 and FDR<0.1 and PCT 20 (in %, minimum percentage detected in respective phenotype) for each subtype of epithelial cells in the MIBC tumors is shown in Gene Sets 2-6). The fibroblasts encompassed 4 major populations defined by key cancer-associated fibroblast (CAF) markers, including fibroblast activation protein (FAP), alpha smooth muscle actin (αSMA, ACTA2), podoplanin (PDPN), and platelet-derived growth factor receptor beta (PDGFRβ) (FIG. 7G, 7H). The immune compartment contained a diverse collection of cells including T-cells, dendritic cells, macrophages, and B-cells as defined by classic immune marker genes (FIGS. 7I, 7J).

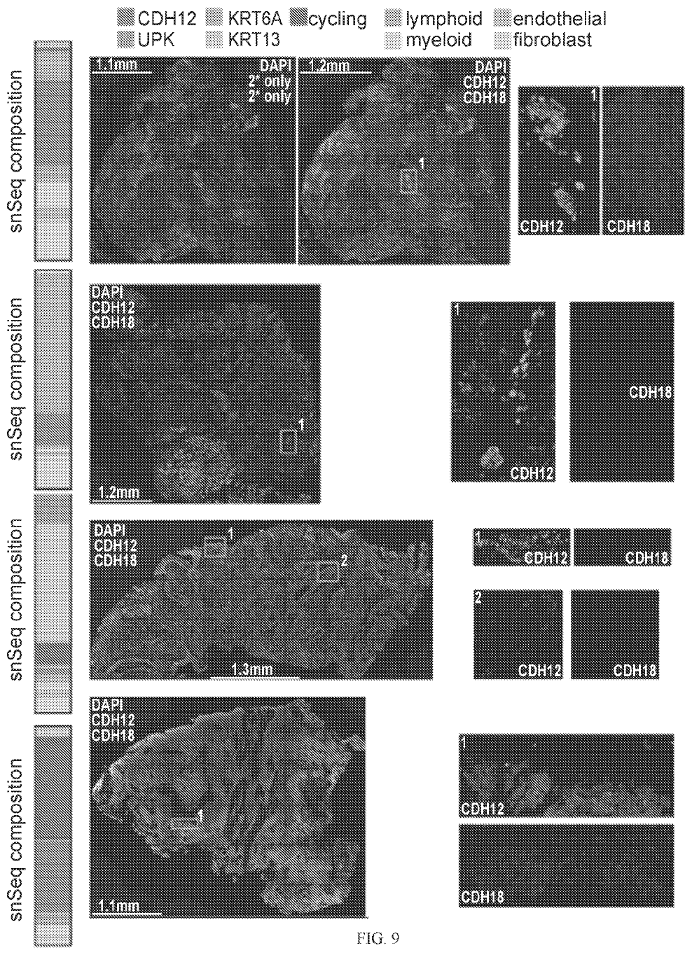

We focused on a deeper analysis of the epithelial compartment as it constituted the bulk of the tumor. Immunohistochemistry verified the expression of KRT13 and KRT17, and CDH12 and CDH18, in tumors that were predicted by snSeq to have high versus low levels of KRT13 and CDH12 epithelial populations, respectively (FIGS. 8 and 9, respectively). We then evaluated the epithelial populations in the context of previously published MIBC gene signatures to determine similarities and differences. The KRT13 and the UPK populations were most closely related to the luminal phenotype, while the KRT6A population was similar to the basal phenotype. Interestingly, the CDH12 population had elements of the p53-like and immune-infiltrated phenotypes indicating that it may be present to some degree in multiple previously established subtypes, and that prior methods (Choi, W. et al., Cancer Cell (2014) 25, 152-165; Seiler, R. et al., Eur. Urol. (2017) 72, 544-554) were unable to fully elucidate its molecular contribution to MIBC. The KRT13 and the UPK populations were the only two that lacked the gene signature derived from immune-infiltrated MIBC, indicating that tumors that are enriched for these populations represent immunologically “cold” tumors (FIG. 1E). Further cross-referencing to conventional uroepithelial differentiation-related markers indicated that the KRT13 and the UPK populations represented a more differentiated phenotype, while the CDH12, the KRT6A, and the cycling populations represented an undifferentiated or dedifferentiated phenotype (FIG. 1F).

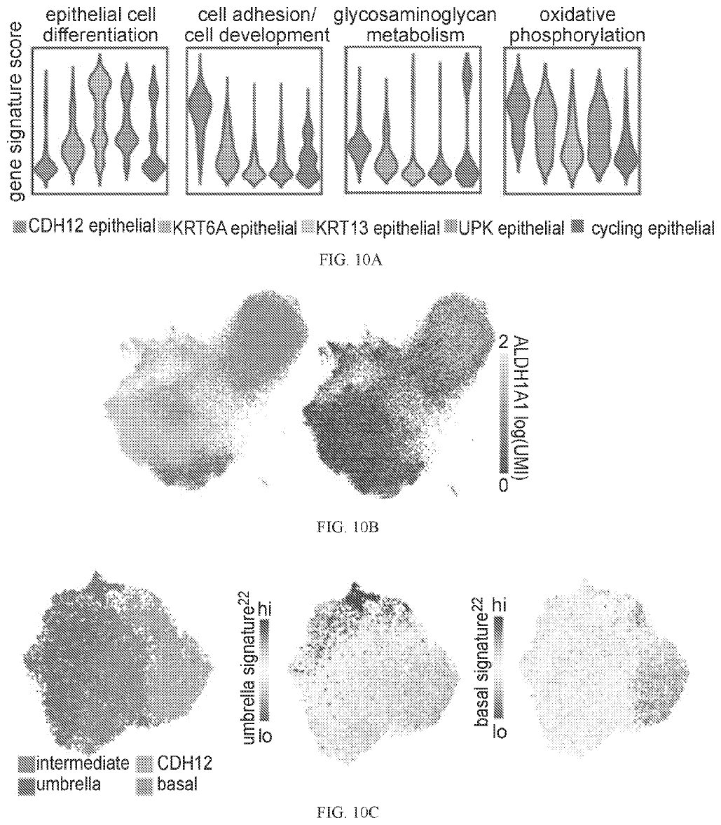

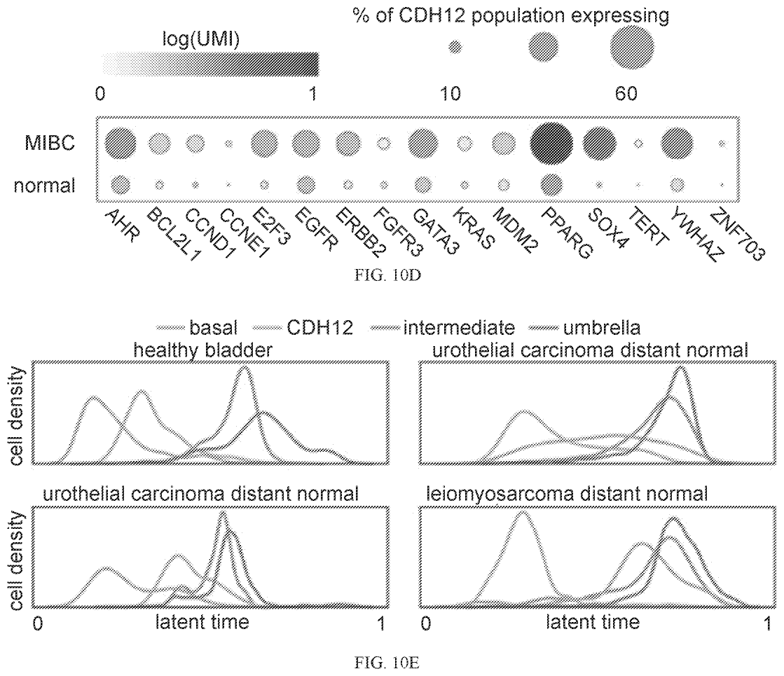

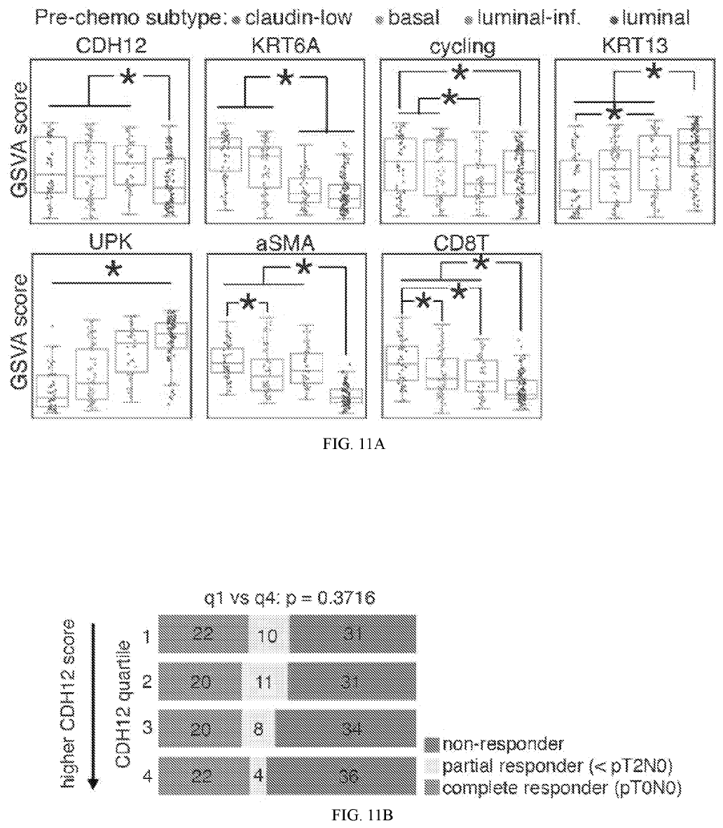

To further characterize the epithelial populations, we performed several unbiased analyses. We constructed a gene network consisting of variably expressed genes with high pair-wise correlations, and used gene ontology enrichment to understand the function of the resultant subnetworks. Consistent with the data in FIG. 1F, the KRT13 and the UPK populations expressed an epithelial cell differentiation network (FIG. 1G). Further underscoring the unique nature of the CDH12 population, we found these cells express cell adhesion and cell development pathways. Gene expression scoring for the identified subnetworks showed significant enrichment in the corresponding epithelial populations (FIG. 10A). The CDH12 population was also analyzed to exhibit high activity of several development-related transcription factors, including NANOG, eomesodermin (EOMES), paired box protein PAX1, and HOXD9, based on Single-Cell rEgulatory Network Inference and Clustering (SCENIC) analysis (FIG. 1H). In contrast, the UPK and the KRT13 populations exhibited higher activity of the differentiation regulators PPARG and GATA3 (FIG. 1H). The CDH12 and the cycling populations also scored highly for stem-like (teratoscore/pluritest) and neuroendocrine gene signatures (FIG. 1I). Consistent with a stem-like phenotype, we also found that the CDH12 population differentially expressed ALDH1A1, a key bladder stem cell marker (FIG. 10B).

CDH12-Enriched Cells are Found in Healthy, Normal Bladder Epithelium.

To gain insights into the biological origin and differentiation path of the newly identified epithelial populations, we performed snSeq profiling on 4 histologically normal bladder samples. Unsupervised clustering of the epithelial cells identified basal, intermediate, and umbrella populations (FIG. 2A and FIG. 10C), similar to previously described in Yu, Z. et al. J. Am. Soc. Nephrol. (2019) 30, 2159-2176. Interestingly, the CDH12 population was clearly distinct from these latter canonical groups, while the intermediate cells expressed the highest levels of KRT3 and KRT17 (FIG. 2B). In addition, the CDH12 population from these samples expressed lower levels of genes known to be amplified in bladder cancer compared to their MIBC counterpart, including TERT and SOX4 (FIG. 10D). We applied RNA velocity analysis to each sample individually, using information about the expression of genes at the unspliced and spliced level to predict a pseudotime trajectory. This identified a trajectory that initiated in basal cells and subsequently diverged into two differentiation paths: one traveling through the CDH12 population and one that skips the CDH12 population. Both paths ultimately converge on the intermediate population and terminate in the umbrella population (FIG. 2C, 2D). Key uroepithelial differentiation markers tracked along this path, with high expression of CD44 at initiation, followed by KRT13 and KRT17 in the middle, and UPK1A, GATA3, and PPARG at the terminus (FIG. 2E). Pseudotime trajectories of all 4 normal samples exhibited similar paths, with the CDH12 population situated near the initiation (FIG. 2F, top, and FIG. 10E). Taken together, this demonstrated that the CDH12 population was a distinct node in the path of bladder differentiation. Transformation at this juncture would lead to tumor development with an enrichment of the CDH12 population.

To determine the transcriptional similarity between the CDH12 tumor cells and their normal counterparts and infer their position along the normal epithelial differentiation trajectory, we identified the nearest normal cell neighbor of every MIBC epithelial cell, using expression similarities, and then assigned the corresponding normal latent times to the tumor cells (FIG. 2F, middle). This revealed that the CDH12, the cycling, and the KRT6A populations were most consistent with an undifferentiated or dedifferentiated phenotype, while the UPK population was most consistent with a fully differentiated phenotype (FIG. 2F, middle). We then sought to understand the predictive potential of this trajectory as previous studies identified luminal (differentiated) and basal (undifferentiated) signatures as prognostically relevant (Mo, Q. et al., J. Natl Cancer Inst. (2018) 110, 448-459; Sjodahl, G. et al., J. Pathol. (2017) 242, 113-125). We created gene signatures from intervals along our identified differentiation paths and scored 259 samples of previously untreated high-grade urothelial MIBC tumors in The Cancer Genome Atlas (TCGA) for each interval using single-sample gene set enrichment analysis (ssGSEA) (Subramanian et al., PNAS (2005) 102, 43, 15545-15550). Strikingly, the interval score corresponding to the most undifferentiated phenotype predicted poor disease-specific survival (DSS) while the interval score of the most differentiated phenotype predicted better DSS, with the interval scores in between demonstrating a transition between the opposing outcomes (FIG. 2F, bottom).

CDH12 Score Predicts Poor Prognosis in MIBC

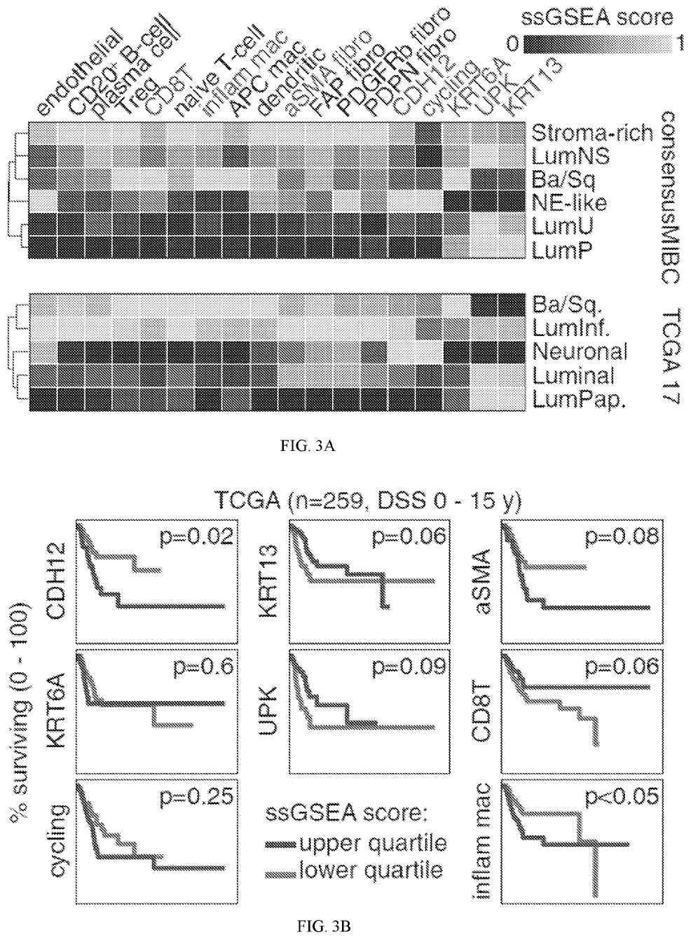

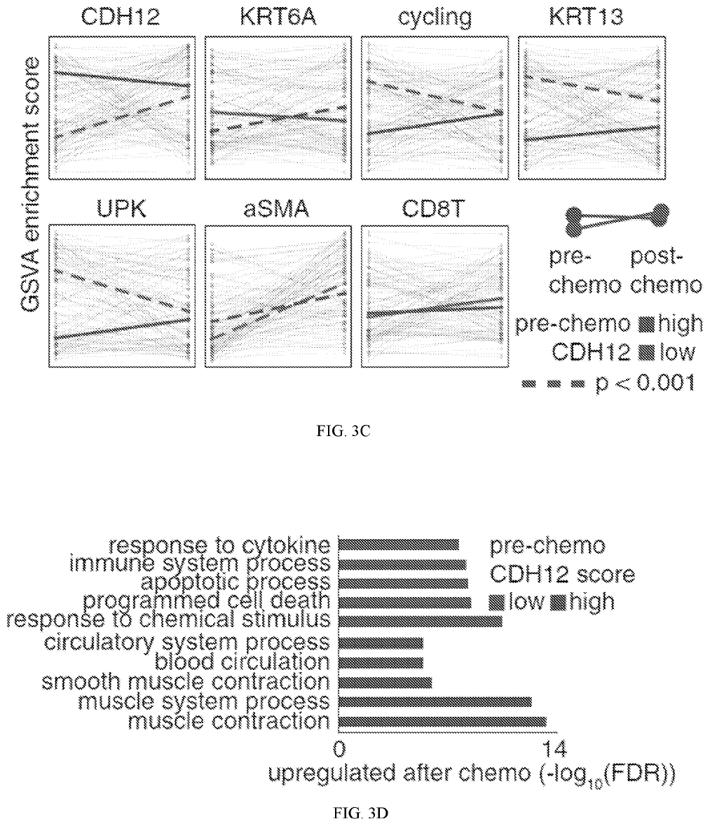

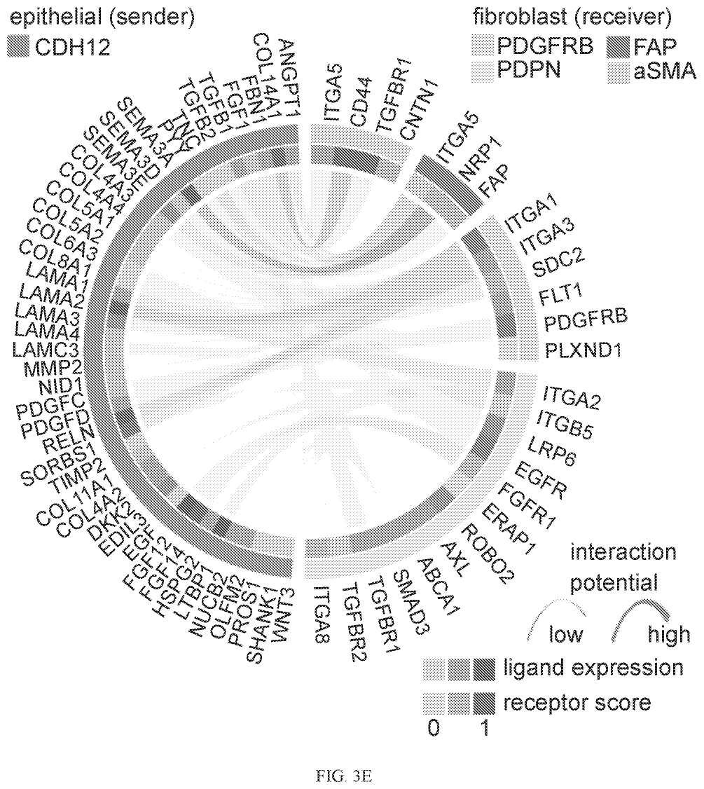

The observed prognostic value of the differentiation path gene signatures and their relationship to the CDH12 population prompted us to delve further into analyzing TCGA high-grade MIBC tumors. We created gene signatures for each of our cellular populations (Gene Sets 12-34) and scored each TCGA sample for these signatures using ssGSEA. We created cellular profiles for each of the TCGA tumors and analyzed them in the context of the consensus MIBC or TCGA 2017 classifications (FIG. 3A) (Robertson, A. G. et al., Cell (2017) 171, 540-556 e525; Kamoun, A. et al., Eur. Urol. (2020) 77, 420-433). We observed good agreement between classification systems. Our UPK signature was enriched in the luminal subtypes, while our KRT6A signature was enriched in the basal/squamous (Ba/Sq) subtypes. Interestingly, speaking to its unique nature, the CDH12 signature distributed across the Ba/Sq, luminal infiltrated, and neuroendocrine-like subtypes, while being notably absent from the luminal papillary (LumP) and luminal uncertain (LumU) subtypes (FIG. 3A). This was consistent with our observation in FIG. 1E that the CDH12 population may be present to some degree in multiple previously established subtypes. The Ba/Sq and the luminal infiltrated subtype, which harbored CDH12 enrichment, also demonstrated enrichment for CD8⁺ T-cells and fibroblasts, which was notably lacking in the LumP and LumU subtypes. The CDH12 and the macrophage signatures were the lone predictors of poor DSS (FIG. 3B). Notably, the KRT13, the UPK, and the CD8⁺ T-cell (CD8T) signatures were linked with better DSS and αSMA fibroblasts with poorer DSS, however these associations did not reach the level of statistical significance.

CDH12 Score Predicts Poor Response to Neoadjuvant Chemotherapy.

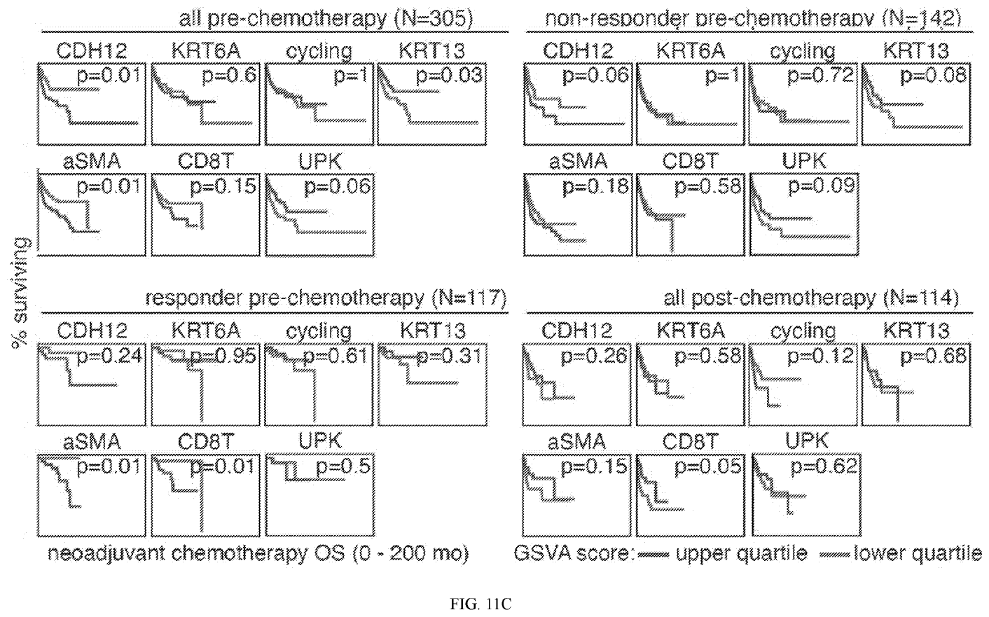

Having established the broad prognostic impact of our molecular signatures on surgically treated MIBC, we investigated their ability to predict response to platinum-based chemotherapy using data from paired pre- and post-NAC bladder cancer samples from a recent study (Seiler R., et al., European Urology, 72, 2017, 544-554, wherein MIBC (cT2-4aN0-3M0) was diagnosed by TUR prior to receiving at least three cycles of neoadjuvant cisplatin-based chemotherapy; Seiler R., et al., Clin Cancer Res, 25(16), 2019, 5082-5093, wherein each patient received at least three cycles of cisplatin-based NAC followed by radical cystectomy). Our gene signatures tracked with the single-sample classifier reported in the study in a manner consistent with the TCGA subtyping (FIG. 11A). While our gene signatures did not predict response rate based on pathological downstaging (FIG. 11B), once again the CDH12 score predicted poor overall survival (OS), while the KRT13 and the UPK (p=0.06) scores predicted better OS (FIG. 11C). To determine how the CDH12 population might associate with changes brought about by chemotherapy, we split pre-chemotherapy samples by high and low CDH12 scores and tracked changes in our gene signatures following chemotherapy. We observed low CDH12 score samples tended to become high CDH12 score samples after chemotherapy, while high CDH12 score samples tended to retain a high CDH12 score after chemotherapy (FIG. 3C). In contrast, the opposite trend was observed when performing a similar analysis using the UPK signature score, while the other epithelial populations did not exhibit any clear progression. This indicates that the CDH12 population is chemo-resistant, while the UPK population is chemo-sensitive. Interestingly, both tumor types increased in αSMA score after chemotherapy, indicating potential stromal activation. Tumors that started with low CD8T scores tended to increase their CD8T score after chemotherapy, indicating immune activation.

CDH12 Cells are Chemo-Resistant and Activate Stroma.

To further understand the changes brought on by chemotherapy in the context of CDH12, we compared gene expression profiles of matched post-chemotherapy and pre-chemotherapy tumors separated by their pre-chemotherapy CDH12 score. Interestingly, tumors that began with a low CDH12 score increased expression of genes related to apoptosis and immune activation in response to chemotherapy, while tumors that started with a high CDH12 score responded to chemotherapy through fibroblast and endothelial cell activation (FIG. 3D). This stromal activation signature prompted us to search for potential communication between the CDH12 epithelial cells and fibroblasts in our snSeq data. Using ligand-receptor interaction analysis, we looked for interactions in which the ligand was differentially expressed by the CDH12 population versus the other epithelial populations and the receiving population demonstrated differential activity of the matching receptor. We observed many significantly enriched interactions between the CDH12 population and fibroblasts, with the most notable being TGFBR1, CD44, and several integrins because of their involvement in cancer-associated fibroblast (CAF) activation (FIG. 3E). TGFβ activates CAFs in a partially CD44-dependent manner, resulting in their proliferation and promotion of the epithelial-to-mesenchymal transition and wound-healing pathways. Taken together, these observations indicate the CDH12 population may represent a chemo-resistant tumor subpopulation characterized by TGFβ-induced CAF activation, while the KRT13 and UPK populations represent chemo-sensitive subpopulations that may undergo apoptosis and induce immune activation through immunogenic cell death pathways.

CDH12 Score Predicts Immunotherapy Response Post-Chemotherapy.

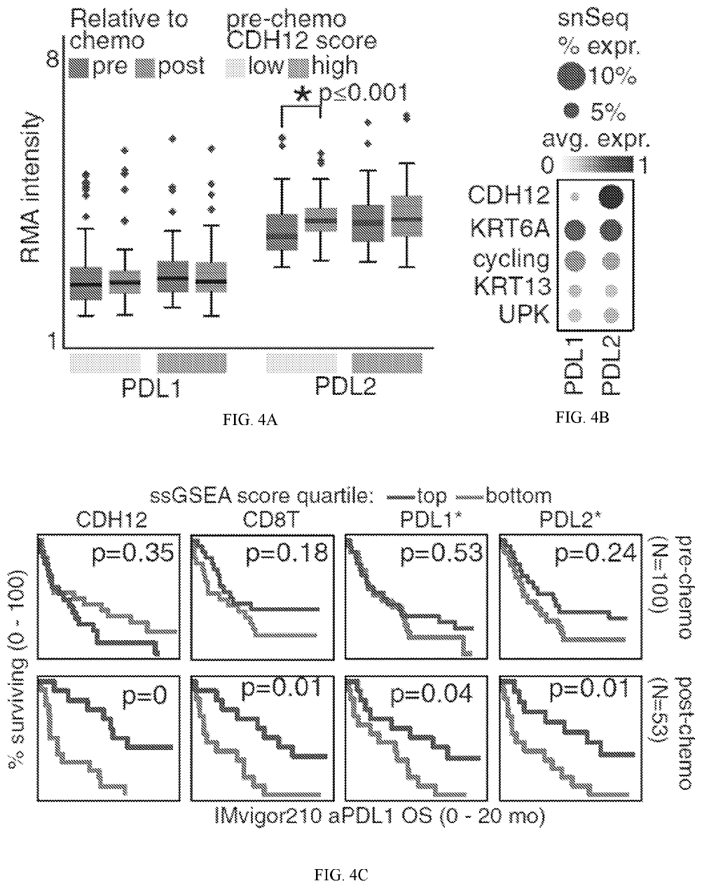

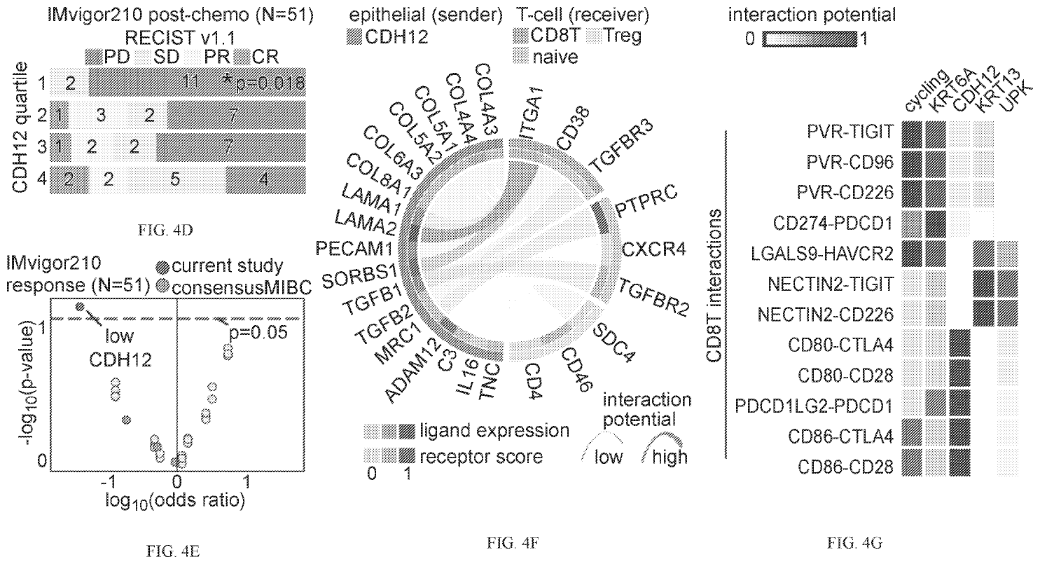

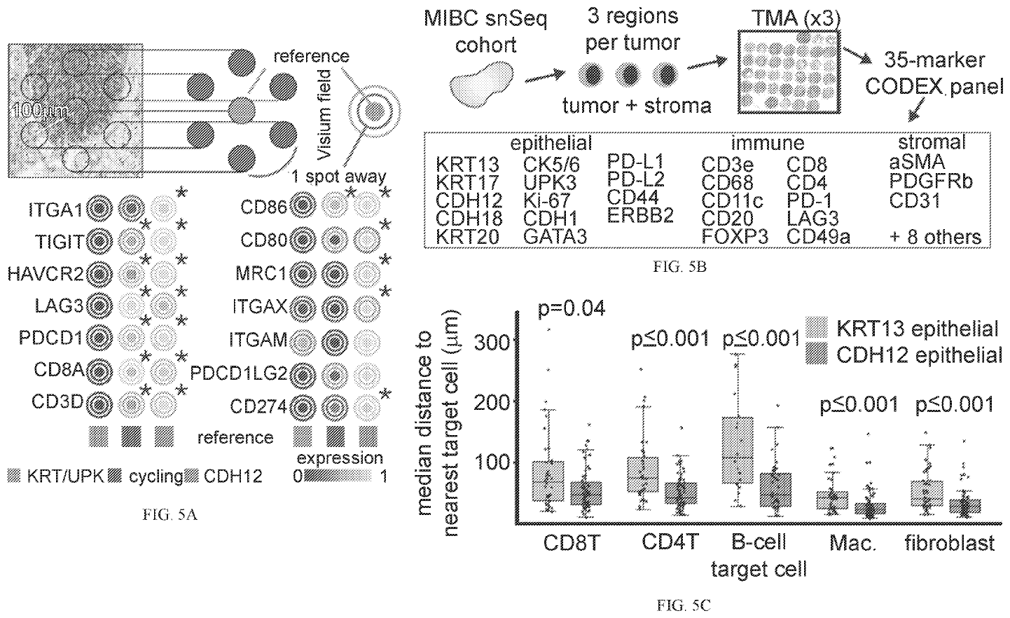

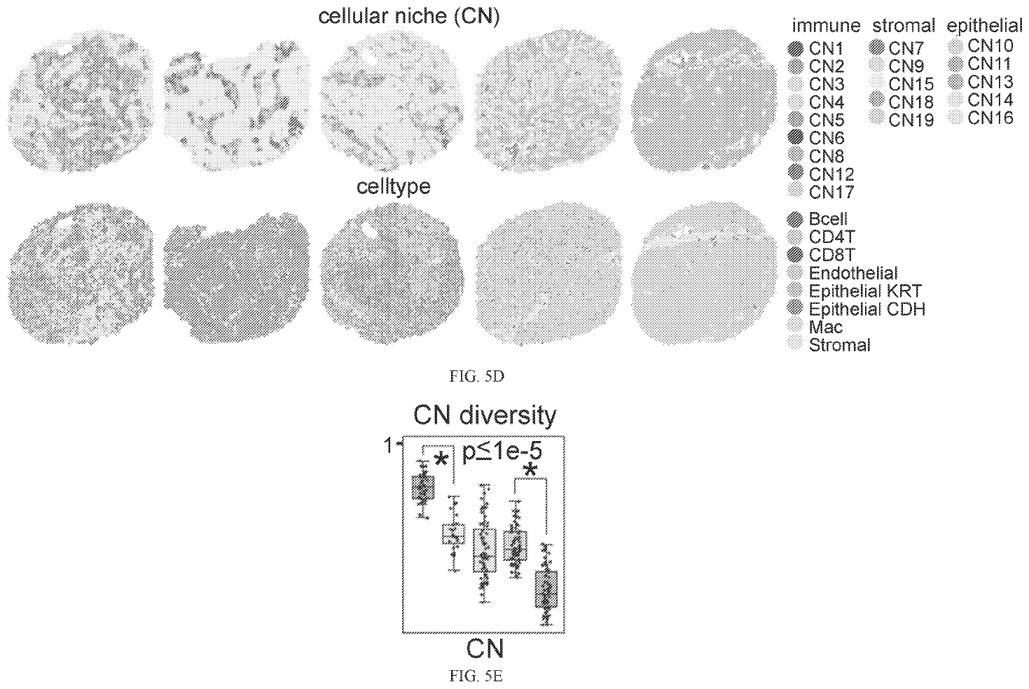

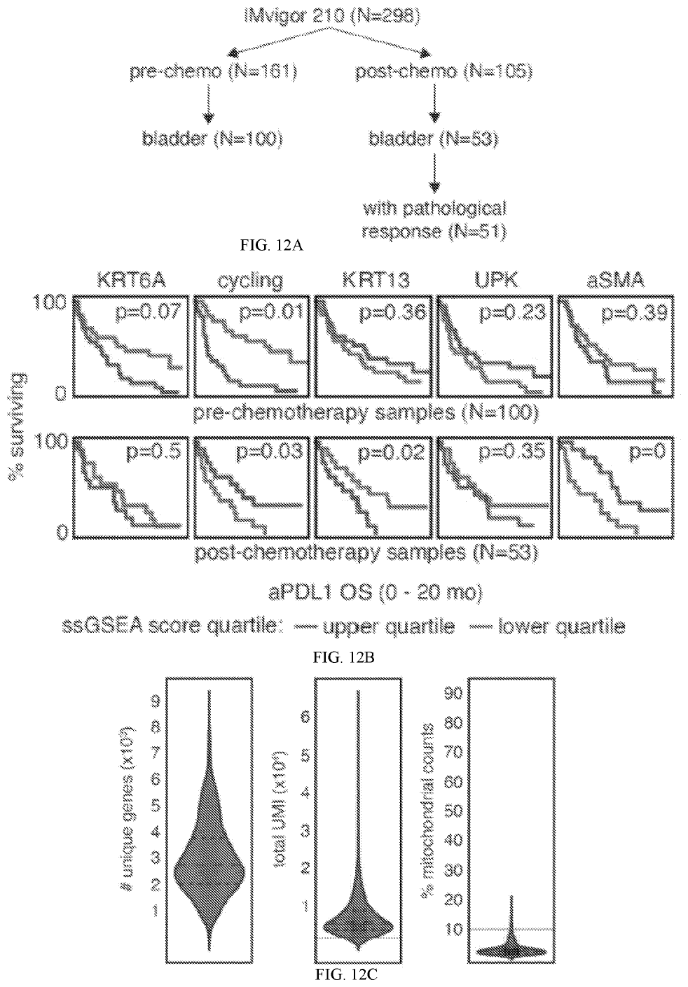

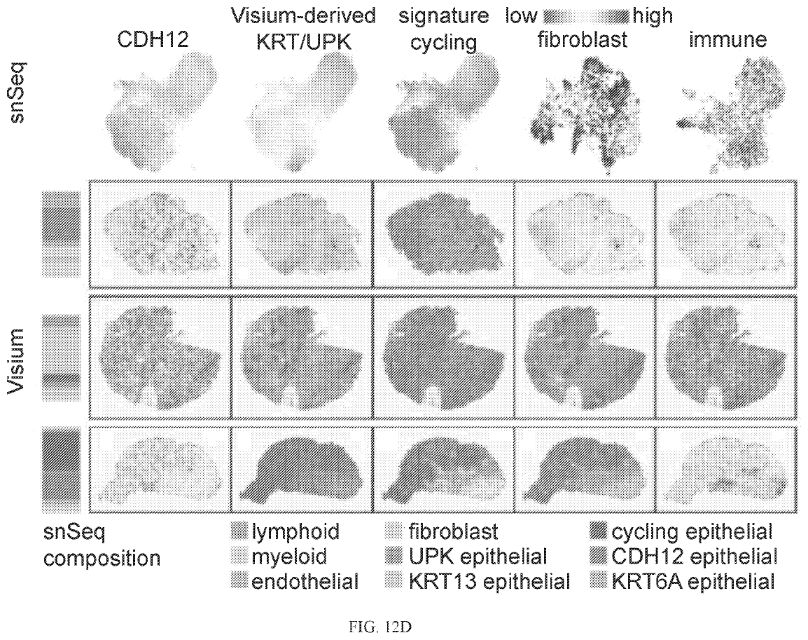

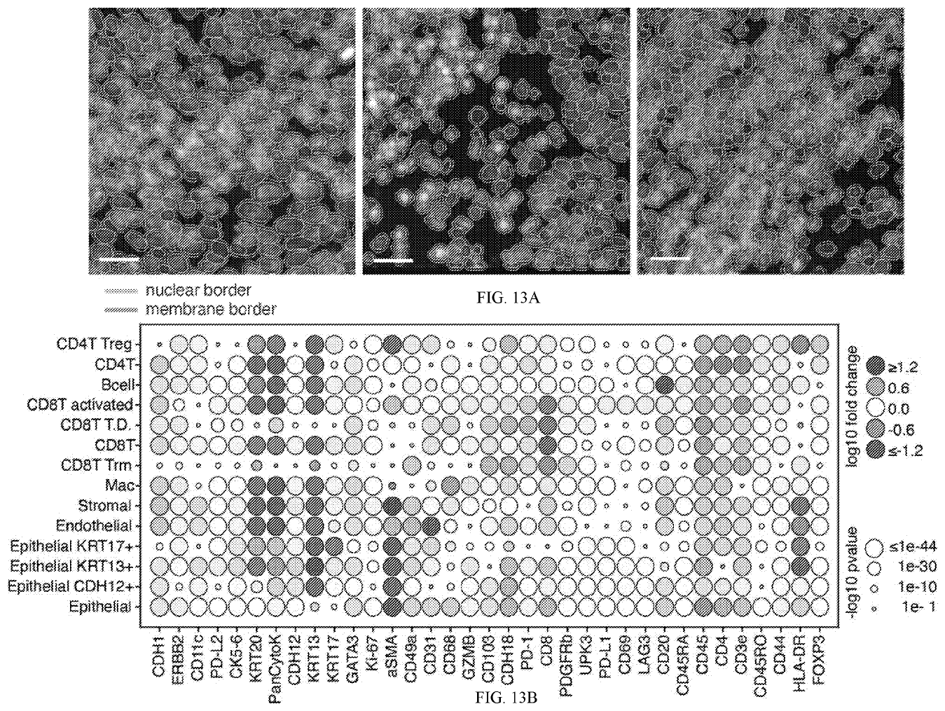

Since tumors with low baseline CDH12 scores responded to chemotherapy with a concomitant rise in their CDH12, apoptosis, and immune activation gene signatures, we also investigated the corresponding changes to immune checkpoint-related genes. With immune activation, we found tumors with low CDH12 scores increased their expression of PDCDILG2 (PDL2) after chemotherapy, while PDL2 expression was higher than PDL1 (CD274) expression in all samples (FIG. 4A). The former observation was consistent with our snSeq dataset showing CDH12 cells expressed the highest level of PDL2 among the epithelial populations (FIG. 4B). This led us to examine our gene signatures in the context of the IMvigor210 trial. This trial investigated, in what the original authors termed Cohort 2 (Rosenberg, J. E. et al., Lancet (2016) 387, 1909-1920), the efficacy of the anti-PDL1 antibody atezolizumab in patients who previously failed to respond to platinum-based chemotherapy. Given our observation that chemotherapy substantially alters tumor composition by enriching for the CDH12 population (FIG. 3C), we split the IMvigor210 cohort into samples originating from bladder which were taken pre-chemotherapy or post-chemotherapy (FIG. 12A). Consistent with the results of the NAC cohort, in the pre-chemotherapy samples CDH12 levels were associated with poor OS, albeit not significantly. Strikingly however, CDH12 levels predicted better OS in the post-chemotherapy samples (FIG. 4C). Scores pertaining to the other epithelial populations as well as the αSMA population exhibited similar differential prognostic values in the pre-versus post-chemotherapy setting, i.e. predicting poor versus better OS in the pre-chemotherapy versus post-chemotherapy settings (FIG. 12B). Furthermore, only in the post-chemotherapy setting did the CD8T score and expression of PDL1 and PDL2 demonstrate significant prognostic value (FIG. 4C). The CDH12 score was also associated with pathological response in the post-chemotherapy setting (FIG. 4D), and indeed it was the only factor with a significant association with response in the post-chemotherapy setting, even when considering the well-established consensus MIBC subtypes (FIG. 4E). Altogether, this indicates that the history of the tumor is important for therapeutic decision-making, as the tumor composition prior to chemotherapy portends the changes that will occur in response to chemotherapy, which then informs prognosis and response for subsequent targeting of the PD1/PDL1 axis.

CDH12 Cells Interact with CD8 T-Cells Through CD49a.

To further understand how the presence of CDH12 cells impacts response to PDL1 blockade, we examined our snSeq cohort for specific ligand-receptor interactions with T-cells. While we again found numerous significant interactions between CDH12 epithelial cells and T-cells, we identified the strongest interaction to be ITGA1, which codes for CD49a, on CD8T (FIG. 4F). CD49a is the alpha 1 subunit of integrin receptors and heterodimerizes with the beta 1 subunit to form a cell-surface receptor for collagen and laminin. The heterodimeric receptor is involved in cell-cell adhesion, inflammation, and fibrosis. CD49a plays a critical role in CD8T migration and surveillance of peripheral tissues. Its blockade or deletion results in impaired accumulation of CD8T in peripheral tissues, indicating that this interaction may partly explain the CD8T persistence in CDH12-high tumors. In a targeted analysis of checkpoint interactions, we identified the CDH12 population as having the strongest PDL2-PD1 (PDCD1LG2-PDCD1) and CTLA-4 interactions with CD8T, while the KRT13 and the UPK populations interacted with CD8T through TIGIT and TIM-3 (HAVCR2) (FIG. 4G).

CDH12 Cells Co-Localize with CD8 T-Cells.

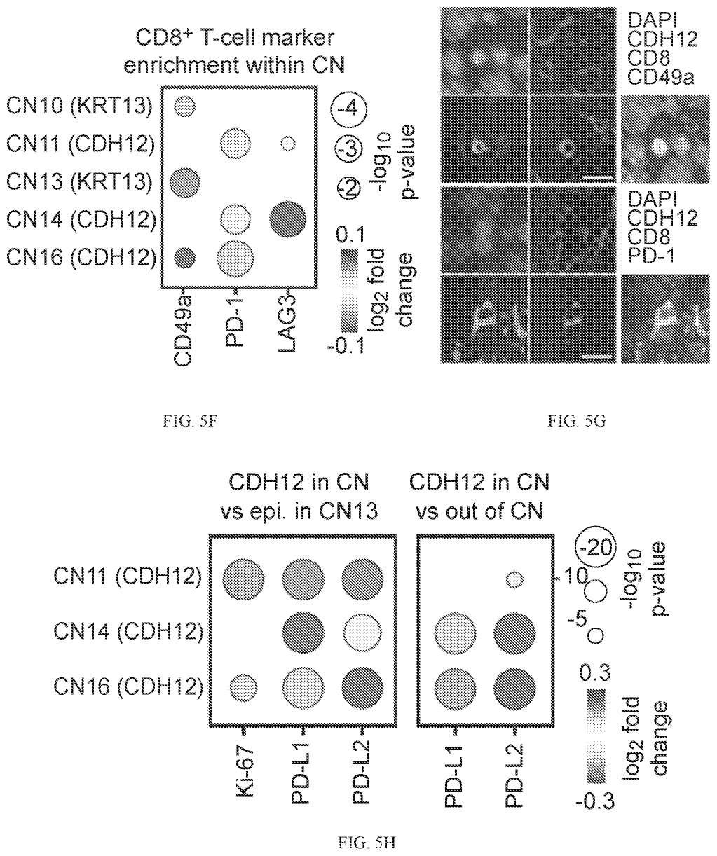

To test the hypothesis that CDH12 epithelial cells attract T-cells, we first used the Visium spatial transcriptomics technology to investigate gene expression localization in tumors from our snSeq cohort. Visium-derived gene signatures closely matched with snSeq expression profiles, and distinct stromal and immune niches were also evident (FIGS. 12C, 12D). Topographic analysis found that areas enriched for a CDH12 signature were also enriched for CD8T with key markers of exhaustion (e.g. gene encoding programmed cell death protein 1 (PDCD1), gene encoding lymphocyte Activating 3 (LAG3), gene encoding hepatitis A virus cellular receptor 2 (HAVCR2)) as well as integrin Subunit Alpha 1 (ITGA1) (FIG. 5A). In contrast, spots enriched for a KRT13/UPK signature exhibited no T-cell gene enrichment.

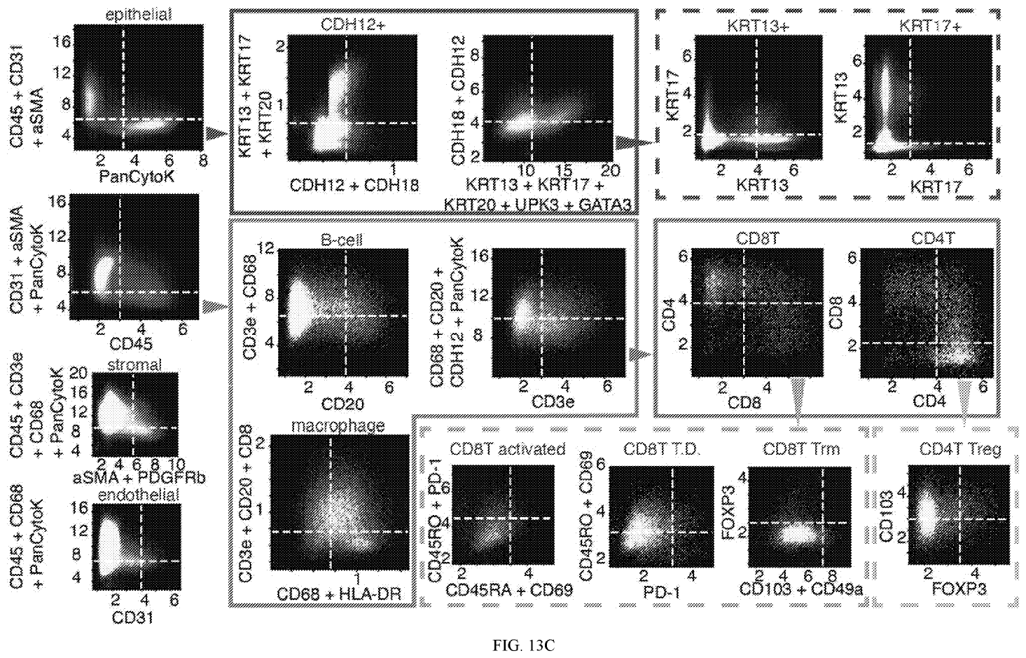

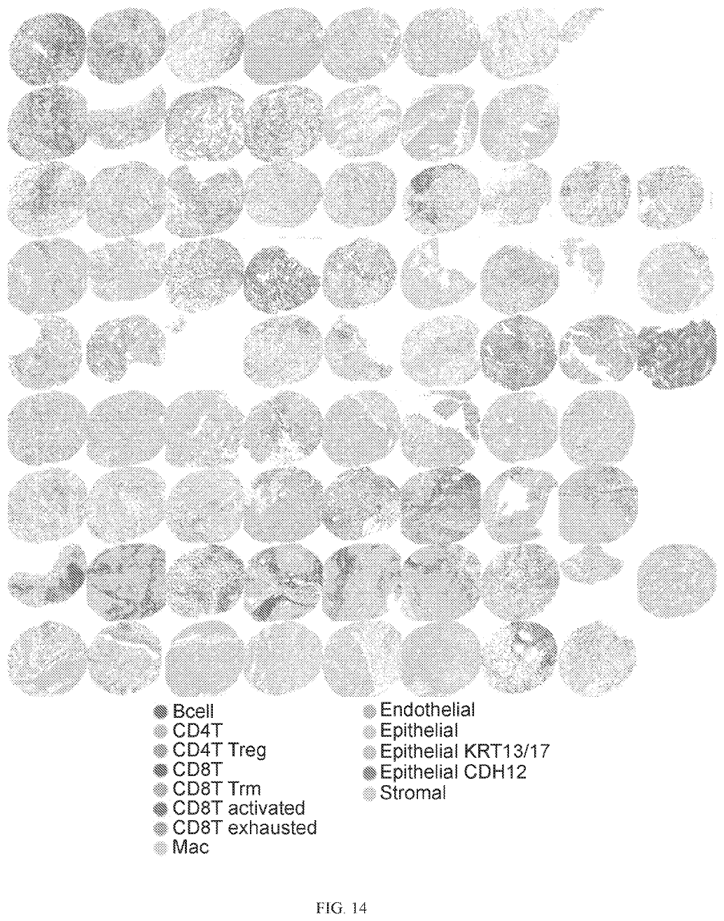

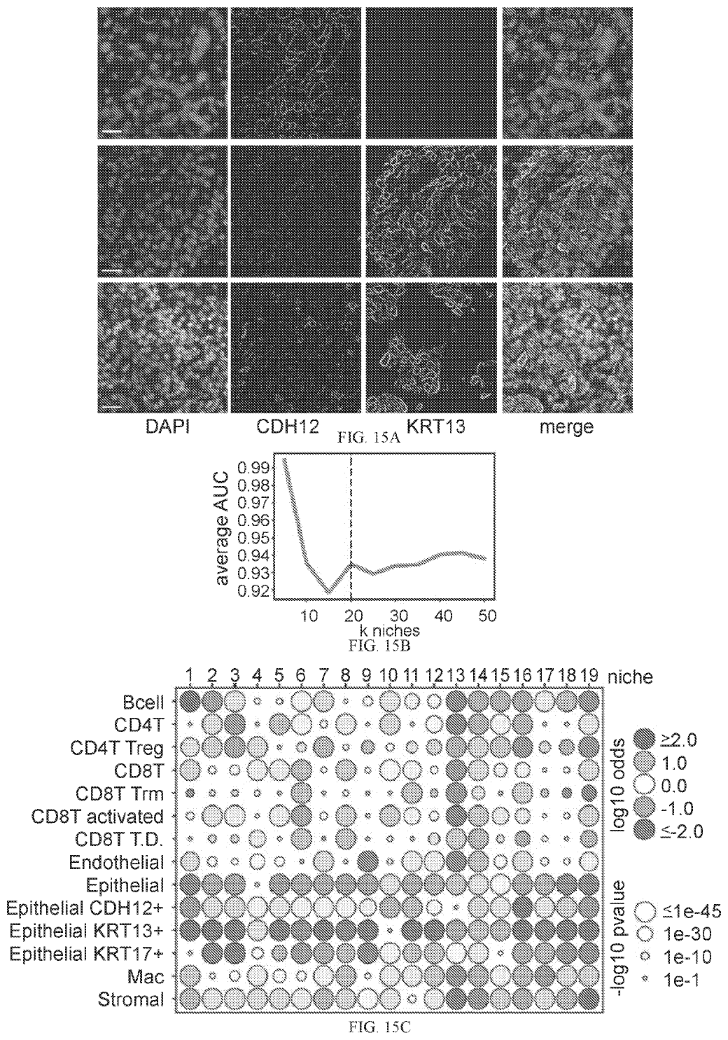

To validate that CDH12 epithelial cells co-localize with T-cells at the single-cell level, we designed and executed a 35-plex IHC panel using the Co-detection by indexing (CODEX) platform on tumor tissue microarrays of the same tumor cohort (FIG. 5B). The tissue areas used in the microarray were specifically selected to harbor both tumor and stroma to allow the study of co-localization of tumor and non-tumor cells. We profiled a total of 75 cores across our patient cohort with ˜360,000 epithelial cells, ˜140,000 immune cells, and ˜90,000 stromal cells passing quality control filtering. We successfully identified all of the major cellular populations including CDH12 epithelial and KRT13 epithelial cells based on expression of CDH12, CDH18, KRT13, and KRT17 (FIGS. 13B, 13C and FIG. 14). We observed that the CDH12 population was significantly depleted for KRT13 expression while the KRT13 population was significantly depleted for CDH18 expression, indicating KRT13 and CDH12 have different co-expression patterns at the protein level (FIGS. 13B, 15A).

CDH12 Cells Define Cellular Niches with Exhausted CD8 T-Cells.

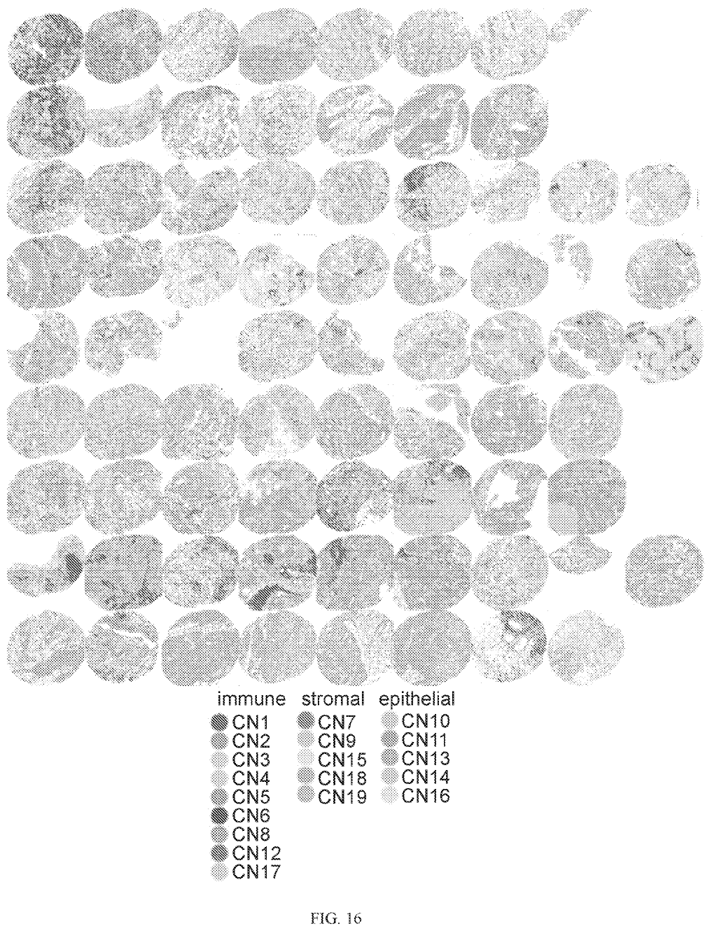

Consistent with our Visium spatial transcriptomics results, we again observed closer proximity of CD8⁺ T-cells to CDH12 epithelial cells than KRT13 epithelial cells using a k-nearest neighbor approach (FIG. 5C). More broadly, CDH12 epithelial cells resided in closer proximity to multiple immune cell types as well as fibroblasts. This indicated distinct spatial distributions for these two different populations. To formally address this, we utilized a cellular niche detection algorithm to identify Cellular Niches (CNs) in an unsupervised fashion (as described in Schurch et al., Cell (2020) 182, 1341-1359 e1319). CNs represent combinations of cell types that frequently co-localize across multiple tumors. Overall, we identified 20 total CNs comprising immune-enriched niches, some of which resembled tertiary lymphoid structures (TLS), stromal-enriched, and epithelial-enriched CNs (FIGS. 15B, 15C and FIG. 16). Within the epithelial-enriched CNs, we identified 3 CNs that were significantly enriched for CDH12 epithelial cells, 2 of which were also enriched for CD8 T-cells. In contrast, we identified 2 CNs where the KRT13 epithelial cells were enriched, and they showed no enrichment for CD8 T-cells (FIG. 5D and FIGS. 15B, 15C). Additionally, the CDH12-enriched CNs were more diverse in terms of their constituent cell types than KRT13-enriched CNs, as assessed by Shannon entropy, a metric for diversity (FIG. 5E). This supported our original observations in that the CDH12 population resided in multiple spatially distinct niches, that were immune-infiltrated whereas the KRT13 population was restricted to niches resembling an immune “desert” phenotype.

We then asked how the identified CNs predict T-cell and epithelial cell phenotypes within them. CD8 T-cells residing within CDH12-enriched CNs expressed higher levels of CD49a (coded by IGA1) (CN16), PD-1 (CN11 and CN14), and LAG3 (CN14) than CD8 T-cells residing in non-CDH12-enriched CNs (FIGS. 5F, 5G). CDH12 cells within all three associated CNs had higher PD-L1 expression compared to epithelial cells in CN13, the most KRT13-enriched CN. In contrast, they expressed lower levels of PD-L2 (FIG. 5H, left). Interestingly, the CDH12 cells also expressed lower levels of Ki-67 compared to CN13, consistent with our snSeq findings and their potentially chemo-resistant nature. Among the three associated CDH12 CNs, CN14 contained CDH12 cells with the highest PD-L1 and PD-L2 expression, and this was consistent with CD8T in this niche having the highest expression of LAG3, which promotes in a tolerogenic state in CD8T and exhaustion with PD-1 (FIG. 5F and FIG. 5H, right). Together, these data support the hypothesis that CDH12 epithelial cells reside near CD8 T-cells in part through CD49a interactions and may promote T-cell exhaustion through PD-L1 and PD-L2. This would partly explain the better response and survival for patients with high CDH12 signature scores when treated with atezolizumab.

In all, we performed the first comprehensive profiling of MIBC at the single-nucleus level, which allowed us to elucidate the constituents of current molecular subtypes and to derive more therapeutically relevant molecular signatures with higher resolution. We identified both known epithelial phenotypes as well as a new CDH12 phenotype that represents a previously undescribed poorly differentiated cellular state. This CDH12-“high” phenotype accurately predicts poor prognosis for patients treated with surgery as well as platinum-based neoadjuvant chemotherapy. It also successfully predicts better prognosis and higher response rates to PD-L1 blockade. We linked the chemoresistance of these cells to a reduced proliferative state, a highly fibrotic and vascularized tumor ecosystem, and expression of the chemoresistance gene ALDH1A1. However, these cells also express high levels of ligands for CD49a as well as PD-L1 and PD-L2, which combine to promote a microenvironment enriched for exhausted T-cells that likely become unleashed and benefit from immune checkpoint blockade. Through an extensive CODEX analysis, we confirmed the spatial proximity of CDH12 cells to CD49a-expressing, exhausted CD8 T-cells within unique cellular niches.

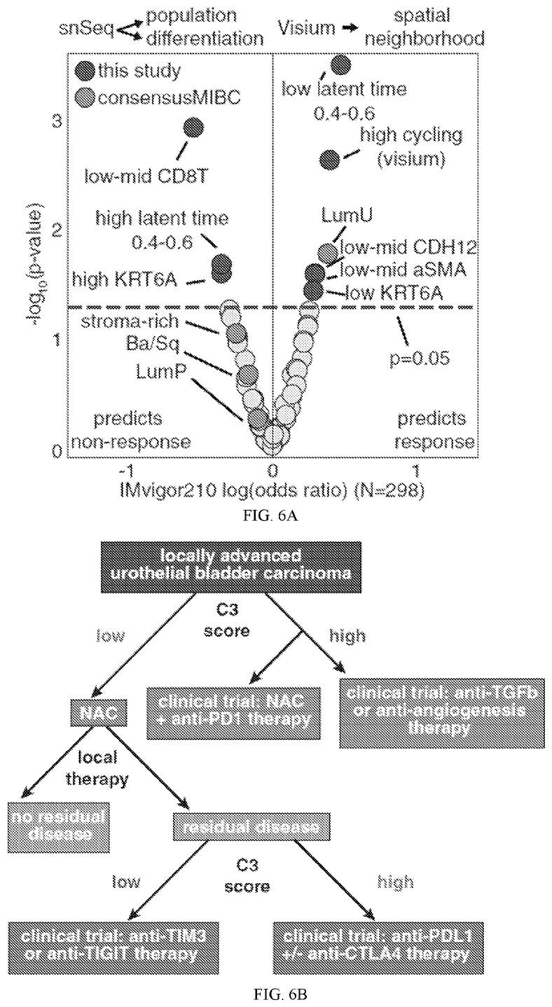

Altogether, we derived gene signatures pertaining to specific cell populations, uroepithelial differentiation, and intra-tumoral spatial neighborhoods that provide superior therapeutic relevance than previous bulk-based subtypes (FIG. 6A). This sub-population is remarkable for the degree it communicates with other cellular types and by virtue of this communication to establish distinct intratumor neighborhoods. Therefore, we can call this the Cell-Cell Communication (C3) subpopulation and use its gene signature score (C3 score), or the relative gene expression profile of the subpopulation, in further studies. Through these findings we speculate that gene expression profiling can serve to triage patients who would benefit from NAC (low CDH12, or low C3 score) (FIG. 6B). Furthermore, these data indicate that anti-TGFβ/anti-angiogenesis strategies could be beneficial in CDH12-high or high C3 score tumors. Residual tumors following NAC with low CDH12 (or low C3 score) might benefit from targeting alternative immune checkpoint pathways such as TIM3 or TIGIT (FIG. 6B) while those with high expression might benefit from single agent or combination ICT. This study paves the way for further analyses of the molecular mechanism used by CDH12 cells (or C3 cells) that lead to such unique predictive characteristics, and potentially for the development of inhibitors to enhance chemotherapy efficacy for tumors with high CDH12 expression (or high C3 scores). It also provides compelling rationale for a number of possible clinical trials based on tumors with high CDH12 expression (or high C3 scores) prior to NAC as well as in patients with residual disease following NAC (FIG. 6B). While the IMvigor 210 trial results indicate that high CDH12 expression (or high C3 scores) post-NAC predicts superior response to atezolizumab, paired pre- and post-NAC samples were not available. Thus, a prospective analysis which profiles how the evolutionary history of the tumor in response to NAC impacts response to atezolizumab would be insightful. We conceive that those tumors which start with low CDH12 and respond to NAC with increases in CDH12 scores would experience the most benefit with atezolizumab, as we showed that this is accompanied by an immune activation that might be prolonged with atezolizumab. Clinical assay development can also address the practical application of a “low” versus “high” C3 score, which would entail establishing absolute standard curves for RNA/protein levels, as RNA sequencing provides a relative quantification. The addition of an IHC-based assay for enumerating C3/CD8T cellular niches similar to the ones we defined with CODEX may also prove useful to investigate the value of our findings in patient stratification for either NAC or checkpoint inhibitor therapy.

Materials and Techniques

Research Ethics.

Urothelial tissue from twenty-five patients with high-grade muscle invasive bladder cancer (MIBC) and 4 patients without bladder cancer were obtained from patients who underwent surgery. All patients provided written informed consent, and no one receive neoadjuvant chemotherapy. All samples were immediately snap-frozen in liquid nitrogen and stored at −80° C. until used. The Research Ethics Committee of Cedars-Sinai Medical Center approved the study (Study00000542).

Tumor and Normal Sample Preparation.

Nuclei were isolated from fresh frozen MIBC tumors using a method modified from a recent single-nuclei RNA-sequencing (snSeq) study (Gaublomme, J. T. et al., Nat. Commun. (2019) 10, 2907). The ST-SB buffer from that study was modified by removing Tween-20 and supplementing with 0.04 U/μL Protector RNase Inhibitor (Roche). Unless otherwise specified, all sample manipulation was performed on wet ice with wide-bore pipet tips (Rainin) and all centrifugations were performed with a swinging bucket rotor maintained at 4° C. for 5 minutes at 850×g. In brief, the frozen tissue was transferred onto a plate on dry ice and crushed into ≤1 mm³ pieces. This was then transferred to a 2 mL dounce homogenizer (Kimble, cat: 885300-0002) on wet ice containing 1 mL of Nuclei EZ lysis buffer (Sigma, cat: NUC101). The tissue was then dounced approximately 20× with Pestle A followed by 20× with Pestle B. The lysis was then quenched by adding 1 mL of ST-SB. The sample was filtered through a pre-wetted 30 μm filter (Miltenyi Biotec, cat: 130-041-407) into a 15 mL conical tube. The homogenizer was rinsed 3× with 1 mL of ST-SB and this was transferred through the same 30 μm filter into the 15 mL conical tube. The sample was then centrifuged, the resulting supernatant removed, and the pellet resuspended with 500 μL of ST-SB. The sample was then passed through a pre-wetted 20 μm filter (Miltenyi Biotec, cat: 130-101-812) into a 1.5 mL protein lo-bind microcentrifuge tube (Eppendorf, cat: 022431081) and centrifuged. At this point, Totalseq hashing antibodies (Biolegend, clone Mab414) were also centrifuged at 14,000×g for 10 minutes at 4° C. The sample pellet was then resuspended in 100 μL of ST-SB and 10 μL of Human TruStain FcX block (Biolegend, cat: 422301) was added. The sample was pipet mixed and incubated at 4° C. for 5 minutes. Then 1.5 μg of the appropriate hashing antibody was added to the appropriate samples, pipet mixed, and incubated at 4° C. for 15 minutes. The samples were pipet mixed once halfway through this incubation. The samples were then washed 2× with 1 mL of ST-SB, pooled appropriately, and filtered through another 30 μm and 20 μm filter. Nuclei concentration was quantified by mixing an aliquot of the sample with DAPI at a final concentration of 0.025 mg/mL in H₂O. Samples were finally processed according to 10× Genomics protocol for the 3′ v3.1 assay and were super-loaded to target of 20,000 nuclei recovery. We observed that nuclei yield less total cDNA than cells, therefore we increased the first cDNA amplification cycle number by 2. Hashing libraries were generated according to the Biolegend Totalseq protocol for the 3′ v3.1 assay. In total, 57 samples from 25 patients were processed.

Nuclei were isolated from histologically normal bladder tissue using the same protocol as above, but without hashing antibodies. Therefore, each sample was run in its own 10× Genomics reaction. In total, 4 samples from 3 patients were processed, with 3 samples originating from patients with urothelial carcinoma or leiomyosarcoma (taken distant from the involved site and verified by a trained pathologist to be uninvolved), and 1 sample originating from a healthy bladder. All samples were sequenced by the Cedars-Sinai Applied Genomics, Computation & Translational Core on a Novaseq to a sequencing saturation of approximately 60%. Samples were processed with CellRanger (10× genomics, v3.0.2) using a pre-mrna reference based on the GRCh38-3.0.0 reference. Hashing libraries were aligned using the Cite-seq-count program (v1.4.3) with the cell barcodes from the CellRanger output as the barcode whitelist. The UMI counts from Cite-seq-count were then used for demultiplexing the MIBC samples using a combination of the Seurat HTOdemux function and a secondary custom script in MATLAB. The secondary script was used to recover nuclei that were identified as negative for all hashtags by the HTOdemux function, but actually passed the minimum number of counts identified by the HTOdemux function for one and only one hashtag. All nuclei that were determined to be doublets or that remained negative after the recovery step were then removed from subsequent analyses. Since the histologically-normal samples were not hashed, putative doublet nuclei were identified using Scrublet (v0.2.1) from the filtered feature barcode matrices produced by CellRanger. Scrublet was run using the 10% highest variable genes, identified using the Scanpy (scanpy.pp.highly_variable_genes function; scanpy v1.5.1), with an expected doublet rate of 10%. Nuclei were scored as candidate doublets by Scrublet and removed if their doublet score exceeded 0.25. Finally, for all samples, nuclei with more than 10% of their UMIs mapped to mitochondrial genes were removed, and the top and bottom 5% of nuclei based on number of unique genes and number of UMI were removed.

Visium Sample Preparation.

Tissue optimization was performed on one representative MIBC sample from the cohort used in this study, and the optimal permeabilization time was determined to be 24 minutes. Then 4 samples were cryosectioned at 0 μm and processed according to the 10× Visium protocol. Samples were sequenced by Illumina to a sequencing saturation of approximately 90%. Samples were processed with SpaceRanger (10× genomics, v1.1.0) using the same pre-mrna reference as for the snSeq data analysis to improve consistency between the two datasets. Visium spots were filtered to have at least 1,250 total UMI and less than 10% of their UMIs mapped to mitochondrial genes. Genes that were not detected in at least 4 spots were removed.

Public Bulk RNA-Seq Datasets: TCGA, IMvigor 210, Neoadjuvant Chemotherapy (NAC).

Bladder urothelial carcinoma Illumina Hi-Seq counts from The Cancer Genome Atlas (TCGA) were downloaded from the Genomic Data Commons (GDC) data portal, and corresponding clinical annotation including survival information was accessed via the TCGA Clinical Data Resource (Liu, J. et al., Cell (2018) 173, 400-416 e411). Consensus MIBC classifications of TCGA cases were obtained from the consensus MIBC study (Kamoun, A. et al.; Eur. Urol. (2020) 77, 420-433). Only untreated high-grade muscle invasive cases with outcomes were analyzed (N=259). RNA-seq and sample annotations including overall survival from the IMvigor 210 trial were accessed as described in Mariathasan, S. et al., Nature (2018) 554, 544-548. For survival analysis of IMvigor 210, only samples from Cohort 2 which were annotated as originating from bladder in the pre-chemotherapy (N=100) or the post-chemotherapy (N=53) setting were used. For pathological response analysis of IMvigor 210, only samples from Cohort 2 which had pathological response information and were annotated as originating from bladder in the post-chemotherapy setting (N=51) were used. For the comparison of response prediction shown in FIG. 5C, all samples from Cohort 2 of IMvigor 210 with pathological response information (N=298) were used to facilitate comparison with the consensus MIBC results which were previously published using those samples. After Illumina Hi-Seq counts were obtained from the respective repositories, the raw counts were counts-per-million normalized and log-transformed. Affymetrix array data corresponding to a trial of neoadjuvant cisplatin-based chemotherapy in MIBC was downloaded from GEO (GSE124305 and GSE87304). Array data were normalized using the RMA method from the oligo R package (v1.52.1).

Single Cell Dimensionality Reduction, Clustering, and Subtyping.

Dimensionality reduction and cell type assignment were carried out in a two-step process. Tumor and normal cohorts were clustered and subtyped separately. First, all cohort cells were used to fit a single cell Variational Inference model (scVI v0.6.8) (Lopez, R., Nat. Methods (2018) 15, 1053-1058), resulting in a 128-dimensional representation of cell phenotypes. The scVI latent space was further projected into a 2-dimensional space for visualization by Uniform Manifold Approximation and Projection (UMAP, Rapids.ai cuml v0.12.0) (McInnes, L., Healy, J. & Melville, J. UMAP: uniform manifold approximation and projection for dimension reduction. (2018); Nolet, C. J. et al., arXiv.org, arXiv:2008.00325 (2020)). Unsupervised clustering was performed on the scVI latent space via the leiden community detection algorithm (cugraph v0.17, resolution=0.6) and clusters were labelled as broadly epithelial, fibroblast, immune, or endothelial using a panel of marker genes gleaned from the literature. Clusters that could not be clearly annotated as a specific cell type or clusters that expressed combinations of lineage-defining markers that are not known to be co-expressed were removed from further analysis. Each broad cell type was then sub-clustered by again applying scVI and the Leiden algorithm. To identify marker genes for detailed subtyping, differential gene expression analysis was applied between sub-clusters in a 1-vs-all fashion (scanpy, Wilcoxon method). Cell types were assigned based on alignment of top differentially expressed genes with marker gene sets gathered from the literature. Gene set scores from published MIBC subtyping and tumor stem cell studies were evaluated for each epithelial cell by comparing the average expression to that of similar-expression genes (Satija, R., Nat. Biotechnol. (2015) 33, 495-502).

To derive gene sets specific to each cell subtype identified in snSeq we applied differential expression analysis separately within the 3 broad cell compartments (epithelial, fibroblast, and immune). For each compartment, a differential expression test was performed genome-wide for each specific subtype against all others subtypes in that compartment (e.g. KRT epithelial vs CDH12 epithelial, cycling epithelial, etc.). The top 200 up-regulated genes for each subtype according to the scanpy “rank_genes_groups” tool's “score” column were taken as putative markers for that subtype. To break ties in cases when one gene was assigned as a marker to multiple subtypes, the gene was ultimately assigned to the subtype with the higher “score”. These gene signatures are shown in Gene Sets 12-34.

SCENIC Regulon Analysis and Gene Co-Expression Modules.

To interrogate active transcriptional networks within each epithelial cell subtype we performed gene co-expression module analysis and single-cell regulatory network inference and clustering (pySCENIC v0.10.0) (Aibar, S. et al., Nat. Methods (2017) 14, 1083-1086). SCENIC analysis was performed with 6,979 highly variable genes using a curated list of human transcription factors, and cisTarget database scoring motif enrichment up to 10 kilobases up and downstream of transcription start sites. To complete the SCENIC workflow, AUCell scores were calculated for each identified regulon.

Gene co-expression modules for tumor epithelial nuclei were derived from the genome-wide pairwise gene Pearson correlations calculated from library size-normalized, log-transformed counts. Genes were filtered first based on being differentially expressed across clusters (FDR≤0.05 and absolute log fold change≥0.3) and then based on a minimum number (N=5) of correlations above an absolute correlation threshold (Corr=0.4). Genes were clustered according to Pearson correlation and modules were partitioned by hierarchical clustering (scipy, metric=Euclidean). Module genes were queried for Gene Ontology (GO) term enrichment using gprofiler via scanpy (Raudvere, U. et al., Nucleic Acids Res. (2019) 47, W191-w198). For visualization, individual genes were associated to the epithelial cell subtype with maximum expression of that gene.

RNA Velocity and Tumor Nearest Normal Neighbor Identification.

Alignment for RNA velocity analysis was performed using the velocyto package (La Manno, G. et al., Nature (2018) 560, 494-498), and downstream velocity analysis was performed using scVelo (v0.17.15) (Bergen, V., Nat. Biotechnol. (2020) 38, pages1408-1414). The same genome annotation files used for CellRanger were used for alignment, and the GRCh38 repeat mask files were downloaded from the UCSC genome browser. Cells that had previously passed QC and were subtyped in the previous gene expression analyses were extracted from the velocyto output.

Normal epithelial nuclei were analyzed individually with scVelo. Gene expression moments were calculated on the top 5,000 highly variable genes with at least 20 combined counts using the UMAP method. RNA velocity was run using scVelo's dynamical model. Next we sought to find the cell from the normal samples that was nearest to each tumor epithelial cell in gene expression space. The top 500 genes correlating gene expression with the latent time (minimum correlation 0.3) were identified from each normal sample and aggregated (total 1,118 unique genes). Using the library size-normalized, log-transformed counts of these latent time genes we proceeded by comparing each tumor epithelial cell with each normal epithelial cell by calculating the L1 norm of the difference of normalized gene expression. Each tumor epithelial cell inherited the latent time of its nearest neighbor normal cell defined as the normal cell with the minimum L1 norm. Latent time gene signatures were derived by first binning tumor epithelial cells into 5 evenly spaced time intervals according to their predicted latent time. Differential expression was performed to recover the top 200 differentially expressed genes for cells within each time interval versus all other time intervals in a 1-vs-all fashion (scanpy, Wilcoxon method). In the event that a gene appeared in the top 200 for more than one-time interval, the gene was assigned to the signature of the interval with the highest differential expression score.

Ligand-Receptor Interaction Analysis.

Receptor activity scores were based on expression of signaling proteins and gene regulation targets downstream of receptor activation. A curated table of ligand-receptor pairs was obtained from SingleCellSignalR (Cabello-Aguilar, S. et al., Nucleic Acids Res. (2020) 48, e55). We first assembled gene signatures describing receptor activity by collecting protein-protein signaling connections and gene regulatory associations included in the NicheNet graphs. Ultimately, 75 receptors that failed to accumulate signatures of at least 5 genes were excluded from further analysis, leaving a total of 675 receptors, and 2,886 total ligand-receptor pairs to be interrogated. The receptor activity was defined as the average absolute deviation of receptor signature genes from the average expression of those genes in a background composed of the same broad cell type (epithelial, fibroblast, lymphoid, myeloid).

Ligand-receptor interactions were determined based on the expression of the ligand in a sender population of cells and the concurrent activation of the corresponding receptor in a receiving population of cells. To perform a general interaction analysis, we first pooled cells by subtype across all tumor samples. To determine available ligands that were enriched in individual subtypes, we performed differential expression analysis (scanpy, Wilcoxon method) of ligand genes for each subtype against cells within the same broad cell type. Available ligands for a sending population were those that met a minimum log fold change of0.5 and maximum adjusted p-value of 0.05. Similarly, receptor activities were tested for enrichment in each subtype relative to a background of the same broad cell type. Active receptors were called according to a minimum log fold change of 0.25 and maximum adjusted p-value of 0.05). All ligands and receptors were required to be expressed in at least 10% of sending or receiving cells respectively. Candidate ligand-receptor pairs were assessed from the available ligands and active receptor sets. Finally, candidate ligand-receptor pairs were subjected to a spatial co-expression filter. Spatially co-expressed ligand-receptor pairs were determined in the spatial transcriptomics dataset. A ligand-receptor pair was called spatially co-expressed if, within at least 1 tumor, 25% of “spots” exhibiting the ligand expression (UMI>0) also had receptor expression (UMI>0). Ligand-receptor pairs were visualized with Circos plots. Each plot included heatmap tracks of standardized ligand expression in one sending subtype and standardized receptor activity in several receiving subtypes. Interaction potential was defined as the product of average ligand expression with average receptor score and visualized as links connecting ligand to receptor. Ribbon transparency was determined by the scaled interaction potential according to transparency=min(0.9, 1−(potential/potentialmax)²) so that the highest potential interaction was the least transparent and a maximum transparency of 90% was imposed to ensure all ribbons were visible.

ssGSEA, Kaplan-Meier Analysis, and Differential Gene Expression for Bulk RNA-Seq.

Gene set enrichment of the tumor single cell subtype signatures and latent time signatures was assessed in each of the bulk RNA-seq samples from the TCGA and IMvigor 210 cohorts, and in the Affymetrix array data of the Black cohort. TCGA and IMvigor 210 samples were scored by single sample Gene Set Enrichment Analysis (ssGSEA, package GSEApy v0.10.1). The neoadjuvant chemotherapy cases were scored with Gene Set Variation Analysis (package GSVA v1.36.2). Samples within each cohort were grouped by score quartiles and Kaplan-Meier survival plots were fit using the right-censored overall survival or disease-free survival times (lifelines version 0.25.4). Significance was assessed between the survival curves of the first and fourth quartiles using a log-rank test. Differential gene expression analysis for the neoadjuvant chemotherapy dataset was performed using the limma R package (v3.44.3).

Spatial Gene Signatures and Association with T-Cell Exhaustion Markers.

Gene co-expression modules for the visium spots were obtained in a similar fashion as for the snSeq epithelial analysis, however in this case differential gene expression analysis was performed on each sample using the SpatialDE package (v1.1.3) (Svensson, V., Nat. Methods (2018) 15, 343-346) and genes with FDR<0.05 were combined across samples. Then the same cutoffs from the snSeq analysis were applied except the fold change cutoff was removed. The resulting gene co-expression modules were then annotated based on their relation to the snSeq dataset, e.g. the module whose gene signature was enriched in the CDH12 nuclei was labeled as CDH12-enriched.

Visium field expression profiles (FIG. 4G) were generated by taking the top 5th percentile of spots for a given module as the reference spots, and then averaging the expression of spots in rings around the reference spot. The coordinates for the ring are as follows: (x−(k+1)),(y+(k+1)); (x−(k+1)),(y−(k+1)); (x),(y+(k+2)); (x),(y−(k+2)); (x+(k+1)),(y+(k+1)); (x+(k+1)),(y−(k+1)); where (x,y) are the coordinates for the reference spot and k is the number of spots away from the reference. The figure shows the average of these profiles across all of the reference spots considered and standardized across the modules.

Visium spots were tested for concurrent enrichment of expression profile scores and gene expression by contrasting spots in the top 5^{th} and bottom 5^{th} percentile of module scores. A contingency table was constructed by counting the number of spots with gene expression in the top 5th and bottom 95th percentile and Fisher's exact test (scipy v1.4.1, fisher_exact, one-sided) was performed on the contingency table.

Immunohistochemistry.

Immunohistochemistry was performed on sections taken from FFPE blocks that were made from adjacent pieces of the same tumors from the snSeq cohort. Briefly, sections were deparaffinized and rehydrated, antigen retrieval was performed using a pressure cooker and 1× Universal HIER buffer (Abcam, cat: ab208572), then blocked in protein blocking buffer (Abcam, cat: ab64226) for 1 hour at room temperature. Sections were then washed and incubated with primary antibodies at 4° C. overnight. The primary antibodies used were as follows (all dilutions were performed with protein blocking buffer): KRT13 (Abcam, cat: ab239918, clone EPR3671, 1:100), KRT17 (Abcam, cat: ab212553, clone KRT17/778, 1:100), CDH12 (LSBio, cat: LS-B11408-100, rabbit polyclonal, 1:100), and CDH18 (Thermo-Fisher Scientific, cat: H00001016-M01, clone 6F7, 1:50). Sections were then washed and incubated with the appropriate fluorophore-conjugated secondary antibodies at room temperature for 1 hour. Secondary antibodies used were as follows (all dilutions were performed with protein blocking buffer): Donkey anti-mouse IgG AF568 (Thermo Fisher Scientific, cat: A10037, 1:500) and goat anti-rabbit IgG AF488 (Thermo Fisher Scientific, cat: A11008, 1:500). Sections were finally washed, mounted with Vectashield containing DAPI (Vector Laboratories, cat: H-1200), and imaged using a Leica DMi8 equipped with a Lumencor SOLA SE U-nIR LED and Hamamatsu Orca Flash 4.0 v3.

Co-Detection by Indexing (CODEX) of MIBC Tumor Microarrays.

Tumor microarrays (TMAs) were prepared from 1 mm punches taken from FFPE blocks that were made from adjacent pieces of the same tumors from the snSeq cohort. If possible, 3 punches were taken from each tumor with 1 punch per tumor, per TMA, resulting in 3 final TMAs. Punches were taken from areas of the tumor that were annotated on H&E to contain both tumor and stroma as annotated by a trained pathologist. Sections from each of these 3 TMAs were then collected onto poly-L-lysine-coated coverslips, which were prepared according to the Akoya Biosciences CODEX protocol. Sections were then deparaffinized and rehydrated, and antigen retrieval was performed in a similar manner to the IHC protocol. Sections were then quenched for autofluorescence using a protocol adapted from Du et al. Subsequently, sections were stained and imaged according to the Akoya Biosciences CODEX protocol. Imaging was performed using a Leica DMi8 equipped with a 20× objective, Lumencor SOLA SE U-nIR LED, and Hamamatsu Orca Flash 4.0 v3.

Primary antibodies were initially screened by performing standard IHC, as above, on MIBC tumor sections to verify positive staining. Primary antibodies were then conjugated to their corresponding barcodes according to the Akoya Biosciences CODEX antibody conjugation protocol. Conjugated antibodies were then titrated by performing CODEX staining on a TMA section using the full panel diluted at either 50×, 100×, 200×, or 400×. The dilution that resulted in the optimal signal-to-noise ratio was determined for each antibody individually.

CODEX Data Pre-Processing.

Images were processed with custom software. To process raw CODEX images, 5 preprocessing operations were applied in this order: extended depth of field (EDOF), shading correction, cycle alignment, background subtraction and tile stitching, described briefly here.

1). An EDOF image was produced from the z-stack for each tile where each position is taken from the z-plane most in focus.
2). The CIDRE method (Smith, K. et al., Nat. Methods (2015) 12, 404-406) of optical shading correction was applied to each channel of each imaging cycle.
3). An image registration transformation was estimated between the first cycle DAPI channel and the DAPI of each subsequent cycle. For each cycle, the registration parameters were saved and applied to all other channels from the same cycle.
4. Blank cycles were used to subtract background from each channel.
5. Finally, neighboring tiles were stitched by applying a registration between the overlapping areas between two tiles. First the two tiles with the best naïve overlap were stitched by applying the appropriate registration shift to one of the tiles. Stitching then proceeded with the next two most nearly aligned tiles, until all tiles were merged. Since each cycle was previously aligned to the first cycle's DAPI channel, the registrations used for tile stitching were estimated once on the first DAPI and reused for subsequent channels and cycles.

To obtain nuclear segmentations we applied a pre-trained StarDist model (Fazeli, E. et al., F1000Res (2020) 9, 1279) to the first cycle DAPI image. The model weights of the 2D 2018 Data Science Bowl model released by the original StarDist authors were fine-tuned using a training set of nuclei imaged on our CODEX platform. A “ring percentage” metric was also developed for relevant markers to differentiate cells expressing the marker from adjacent cells whose masks may contain a portion of the signal from the positive neighbor. For surface markers the assumption was, truly positive cells would display signals in a ring-like morphology, while neighboring cells with overlapping masks would not. To quantify cells exhibiting a ring-like pattern, we defined the “ring percentage” by examining the pixels in a ring around the nuclear segmentation contour, and tallying the percentage of these pixels that were positive for the markers CD45, CD3e, CD8, CD4, CD45RA, CD45RO, CDH12, KRT13, KRT17, CD20, ERBB2, and PanCytoK, defined as intensity greater than 20. Lastly, a whole-cell or “membrane” segmentation was obtained expanding the nuclear segmentation area by morphological dilation, without introducing overlaps in adjacent nuclei. The average intensities under each nuclear mask and membrane mask were extracted for each cell to be used for cell type assignment. A Hematoxylin and Eosin stained slide accompanying each of the 3 TMA's was examined by a pathologist and spots identified as necrotic, or with extensive tearing or cautery artifacts were excluded from further analysis.

CODEX Cell Type Identification.

A multi-step strategy was used to assign specific subtypes to single cells by first gating average marker intensity, then applying a k-Nearest Neighbor (kNN) classifier. First, the initial set of 615,171 segmented cells was filtered for low-quality cells indicating errant segmentations or non-specific staining artifacts with three separate gates: low DAPI intensity (filtered 2,501 cells), low total marker expression (filtered 17,597 cells), and high multiple marker expression (filtered 12,547 cells). Cells were manually gated based on intensity of PanCytoK, CD45, αSMA, CD31, CD20, CDH12, CDH18, CD68, CD3e, CD8 and CD4 into a training set consisting of the broad cell types: Epithelial, Epithelial KRT, Epithelial CDH, Stromal, Endothelial, general CD45+ immune, Bcell, CD8T, CD4T and Macrophage. Further selection based on the “ring percentage” feature described above was applied to filter the gated populations using the applicable markers. For this initial classification, the special “blank” and “saturated” classes were retained. The cells that fell into these categories during this initial classification were dealt with in a later step. To account for imbalance in the training set collected, each category was uniformly subsampled to 2,500 training cells, unless fewer than 2,500 training cells were collected in which case all cells were used for that category. In all, a training set of 32,500 cells was used for initial cell typing. 50 features per cell were used for kNN classification: αSMA, CD45, PDGFRb, CD68, CD31, HLA-DR, UPK3, GATA3, CD3e, CDH18, CDH12, KRT13, KRT17, CK5-6, KRT20, CD20, CD8, CD4 and PanCytoK “membrane” and “nuclei” mean intensity features (38), and all “ring percentage” features (12). Features were scaled with the robust scaling method in scikit-learn to normalize the inter-quartile ranges of each feature. A kNN classifier (cuML, version 0.17) was trained on the whole training set using 200 neighbors and uniform weighting. Cells initially classified as CD8T or CD4T were next used in a second phase of T-cell specific gating to identify activated CD8T (CD45RA^{hi}, CD69^{hi}/CD45RO^{lo}, PD-1^{lo}), terminally differentiated CD8T (PD-1^{hi}/CD45RO^{lo}, CD69^{lo}), resident memory CD8T (CD49a^{hi}, CD103^{hi}/FOXP3^{lo}), and regulatory CD4T (FOXP3^{hi}/CD49a^{lo}, CD103^{lo}). In keeping with the aforementioned class balancing procedure, up to 500 cells from each Tcell subset were randomly selected for training, and up to 500 CD8T and CD4T cells not included in the specific subtyping were also included. Thus, a total of 2,445 cells were used for training a second T-cell specific kNN classifier with 100 neighbors.

The final phase of subtype classification was to assign subtypes to those cells still labelled “blank”, “saturated”, or non-descript “Immune”. All cells with a final subtype were used as potential training cells for 10 rounds of classification. Each round, 500 of each subtype were randomly selected as training cells for a kNN classifier with 20 neighbors. The rescued cells were assigned the most frequently predicted subtype across the 10 rounds. Rescued cells assigned to non-immune subtypes were accepted, however rescued immune cells were rejected and filtered from the dataset. Finally, Epithelial KRT13+ and KRT17+ cells were selected by manually gating KRT13 and KRT17 intensity from all classified Epithelial cells. Ultimately, 598,327 cells were assigned a celltype and subtype annotation and included for further analysis. Marker intensity was visualized using a dot plot where the hue of the dots represented the log fold change of that marker in a particular subtype versus all other cells, and the size of the dot represents a Wilcoxon test p-value (scipy, version 1.6.0).

CODEX Niche Detection and Spatial Analysis

Niches were identified according to the subtype distribution of the k=10 nearest cells, with a maximum distance of 200 in image coordinates. Each cell's neighborhood profile was tallied as the percentage of each broad cell type (Epithelial, Epithelial CDH, Stromal, Endothelial, Macrophage, Bcell, CD8T and CD4T) within each cell's 10 nearest neighbors by Euclidean distance, and including the reference cell's celltype. A cellular niche (CN) represents groups of cells with similar neighborhood profiles. Using an iterative classifer-based approach we identified an optimal number of CN's. A k-means clustering (cuML, version 0.17) was performed with several values of k. For each k value, all cell niches were clustered, then divided into 2 training and ½ hold out partitions, then a logistic regression classifier (cuML, version 0.17) was fit on each CN in a 1-versus-all fashion. The area under the reciver operating characteristic curve (AUC) for each of these classifiers was evaluated using the held out partition. The average AUC for each k was plotted. The value k=20 was chosen as a value providing a reasonable number of niches with good individual predictability. The 1-vs-all logistic regression model coefficients were used to assign labels based on predictive cell types for each niche. Two niches with similar composition were merged, yielding 19 final CN's for further analysis. Subsequently, the specific subtype membership within each CN was examined using a Fisher's exact test.

The cellular niche diversity was defined as the Shannon entropy (Eq. 1) of the cells composing a CN, i.e. the cells assigned to the CN, and all of the cells included in computing those neighbor profiles. Only unique cells were considered. For a set of CN cells consisting of n subtypes, P(xᵢ) represents the frequency of the ith subtype amongst the set, and the Shannon entropy is given by Eq. 1). A large value of Shannon entropy indicates diversity in the cell subtypes, whereas a low value indicates a lack of diversity, or that the CN is dominated by a few subtypes.

S=−Σᵢ₌₁ⁿP(xᵢ)log P(xᵢ) (Eq. 1)

Relative marker enrichment between CN's was evaluated with a Wilcoxon test of marker intensity on a specific subtype of cells residing within a particular CN compared with intensity on a subtype of cells residing in another CN. Lastly, direct spatial proximity between two cell types was evaluated per spot as the median distance between each instance of a query cell type to the nearest instance of a target cell type. A Mann-Whitney test was used to assess a difference in these distances across all spots in all TMA's. In all analyses, only spots with at least 25 examples of all cell types, subtypes, or CNs being examined were evaluated.

Code Availability.

Software packages, notebooks and scripts used for analysis are available at github.com/KnottLab/bladder-snSeq. Custom MATLAB code for CODEX preprocessing is available at github./com/KnottLab/codex. The corresponding DOIs are as follows, analysis scripts: doi.org/10.5281/zenodo.5115212, and CODEX preprocessing: doi.org/10.5281/zenodo.5115210.

Data availability. Single-nuclei RNA-seq and HTO data have been deposited in the GEO database under accession code GSE169379. Visium data have been deposited in the GEO database under accession code GSE171351. CODEX processed data are available through figshare: figshare.com/s/4610a15363c8306dfa36, figshare.com/s/2005255a8b65de23109f, figshare.com/s/1d8c7ed76d4b3222ada4). The following datasets are publicly available. Bladder urothelial carcinoma Illumina Hi-Seq counts from The Cancer Genome Atlas (TCGA) were downloaded from the Genomic Data Commons (GDC) data portal, and corresponding clinical annotation including survival information was accessed via the TCGA Clinical Data Resource. Data from the IMvigor210 trial were obtained from the IMvigor210CoreBiologies R package, made freely available by the authors of the trial manuscript. Affymetrix array data corresponding to a trial of neoadjuvant cisplatin-based chemotherapy in MIBC was downloaded from GEO (GSE124305 and GSE87304). The remaining data are available within the Article, Supplementary Information, or Source Data file.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention. While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

TABLE 1
Patient Clinical Characteristics

| Cohort | | | | | | | Tobacco | | | Recurrence | |
|---|---|---|---|---|---|---|---|---|---|---|---|
| ID | Age | Race | Gender | Grade | Invasive | Recurrence | Use | Stage | Dead | Days | Outcome |
| 36 | 84 | B | Male | High | Yes | No | Yes | T3 | 0 | | |
| 54 | 39 | W | Male | High | Yes | | Yes | T2 | 0 | | |
| 72 | 68 | W | Female | High | Yes | No | Yes | T2 | 0 | 270 | |
| 593 | 81 | W | Male | High | Yes | No | Yes | T2 | 1 | 76 | Died |
| 674 | 73 | W | Male | High | Yes | Yes | No | T2 | 0 | 322 | Mets to brain |
| 702 | 46 | U | Male | High | Yes | No | Yes | T4 | 0 | 40 | |
| 739 | 49 | U | Male | High | Yes | No | Yes | T2 | 0 | 521 | |
| 752 | 63 | W | Male | High | Yes | No | Yes | T2 | 0 | 9 | |
| 763 | 69 | W | Male | High | Yes | Yes | Yes | T2 | 0 | 538 | Mets to brain |
| 824 | 67 | W | Male | High | No | No | Yes | T1 | 0 | | |
| 896 | 45 | B | Female | High | Yes | Yes | Yes | T2 | 0 | 510 | |
| 912 | 67 | U | Female | High | Yes | No | No | T2 | 0 | 47 | Pt refused continued |
| | | | | | | | | | | | chemo after |
| | | | | | | | | | | | initial diagnosis |
| 913 | 78 | W | Male | High | Yes | No | Yes | T2 | 0 | | |
| 1246 | 77 | U | Male | High | Yes | Yes | Quit | T2 | 1 | | Declined Adj |
| 1126 | 50 | W | Male | High | Yes | No | No | T2 | 0 | 852 | |
| 1204 | 65 | W | Male | High | Yes | No | Yes | T2 | 0 | 729 | |
| 371 | 63 | B | Female | High | Yes | No | Yes | T3 | 0 | 158 | |
| 419 | 55 | W | Female | High | Yes | No | Yes | T2 | 0 | 54 | |
| 446 | 51 | W | Male | High | Yes | No | Yes | T2 | 0 | 61 | |
| 485 | 78 | W | Male | High | Yes | No | Yes | T2 | 0 | 112 | |
| 489 | 55 | W | Male | High | Yes | No | Yes | T2 | 0 | 1236 | |
| 518 | 71 | W | Male | High | Yes | No | Yes | T2 | 0 | 294 | |
| 59 | 71 | B | Male | High | Yes | No | Yes | T2 | 1 | 178 | Dead |
| 590 | 51 | W | Male | High | Yes | No | Yes | T2 | 0 | 41 | |
| 8 | 82 | W | Male | High | Yes | No | No | T2 | 1 | 150 | Dead |

### Gene Set 1

RBFOX1, CNTNAP2, CSMD1, DLG2, PTPRD, EYS, DPP10, PCDH15, CTNNA3, DMD, MT-CO1, LINC00486, CTNNA2, MT-CO3, FP700111.1, MT-CO2, TMEMI32D, CDH12, GRID2, CSMD3, MT-ND4, CCDC26, CADM2, NRG1, MAG12, CDH18, LRRC4C, ROBO2, CNTN5, AC007402.1, GPC5, LRP1B, ZFPM2, DCC, CALN1, GALNTL6, ANKS1B, KCNIP4, CNTN74, CDH13, MT-ND1, TENM2, CTNND2, TRPM3, NRXN1, C8orf37-AS, MT-ATP6, CNTNAP5, RYR2, SORCS1, ZNF385D, AL589740.1, PRKG1, PTPRT, DLGAP1, CNBD1, PHACTR1, GPC6, AL138720.1, ILIRAPL1, OPCML, RALYL, PRKN, SOX5, ASIC2, AC034114.2, AC011287.1, USH2A, MT-ND3, CACNA1A, EPHA6, ADAMTSL1, MT-ND2, ERVMER61-1, AGBL1, MT-CYB, AC109466.1, MALRD1, DPP6, TBC1D19, NEGR1, NLGN1, DAB1, PCDH9, SUGCT, HPSE2, LINC02240, RGS7, HYDIN, GALNT17, PKN2-AS), SNTG1, AFF3, LSAMP, DSCAM, MT-ND5, CPNE4, FRMD4A, ADGRL2, SGCZ, AC113414.1, DGKB, GRM7, SYN3, AGBL4, NTM, AC090568.2, LRRTM4, MIR4300HG, LINC01090, LINC00276, MDGA2, AL022068.1, ALK, AC058822.1, LINC01317, GRM8, FSTLS, CASC15, AL445250.1, LINC00535, THSD7B, ERC2, NRG3, SGCD, LUZP2, AC016766.1, AQP4-AS1, NKAIN3, AC068633.1, CACNB2, FMN2, AL391117.1, DLGAP2, NCAM2, IL12A-AS1, AL035401.1, ERBB4, HDAC9, KCNQ5, MYO16, PDZD2, RNF219-AS1, SEMA3A, C8orf34, AL603840.1, NAV3, LINC02237, ZNF804B, DNAH9, NPAS3, ESRRG, LINC01830, LINC00534, ADGRL3, AC026316.5, HECW1, RYR1, NUP210L, MIR99AHG, RIPOR2, ATP8A2, MYO3B, MGAT4C, KCNAB1, RELN, GREBIL, DNAH3, NCKAPS, CACNA1C, PLDS, ARHGAP15, RYR3, PCSK5, HCN1, UNC5D, AC009975.1, NELL1, FAM19A1, ASTN2, GRM5, COL14A1, AC008691.1, KHDRBS2, CREB5, LINC00536, ADCY2, XKR4, AC092100.1, UNC79, FAM155A, ATRNL1, GRIN2B, CA10, LHFPL3, LRRC7, TMEM132C, CFAP299, ADGRB3, AF279873.3, CADPS, MYT1L, CN1H3, NOS1, ATP6V0D2, AC068599.1, GSG1L, PIEZO2, CUX2, MIR3681HG, DCDC1, LINC01435, ANK2, PLXNA4, RIMS1, AC010343.3, CELF2, CEP112, PTPRN2, SEL1L2, DOK6, FGF14, ADAMTSL3, PDE1A, AC008415.1, SORCS3, EMCN, SLC14A2, SPTBN4, PLCL1, AC092422.1, MYRIP, NALCN, CUBN, MTUS2, STXBP5L, PPFIA2, AC018742.1, CSMD2, CPA6, FAT3, AC008050.1, COL25A1, COL24A1, LINC02267, KIF6, KLHL1, P7UHD1-AS, AC092650.1, ELMO1, EPHB1, SOX6, AL158198.1, AC068631.1, LAMA2, SLC4A10, LINC00624, GRID1, TARID, PRKCB, AL445584.2, AL357507.1, LINGO2, LINC02511, AC022639.1, LINC00907, LINC02055, CDH4, CCDC178, MEF2C-AS1, ZNF385B, EGFLAM, ST6GALNAC3, DGK1, AC008892.1, SDK2, SPATA16, CLVS1, TNR, U91319.1, LINC01358, LINC01524, AC012409.2, UNC13C, GRIK2, LINC01122, PIK3C2G, GABRB1, AC103409.1, VEPH1, PDE4B, UNC5C, GNG7, RPH3A, DNAH8, AP003181.1, SF3A3, PRUNE2, AK5, AC104461.1, LINC02008, BRINP3, FAM135B, AL512380.2, ZNF536, KCNH5, AC012593.1, AC090833.1, Z96074.1, OTX2-AS1, C15orf53, PTPRO, KCNH7, MCTP1, LINC01322, GRIA4, HMCN1, AKAP6, PPM1E, GPM6A, RXFP1, GRIK1, AC114689.3, RIT2, ITGA8, GABRB3, SAAMSON, AC015522.1, LINC01934, LINC00578, MYO18B, EDIL3, DEPTOR, LINC01609, PAK5, SHISA9, LINC01483, PXDNL, AC084816.1, PDZRN4, LINC00871, BNC2, MMP16, AF241726.2, AC120193.1, EPHA3, TMEM132B, DNAH12, GNAL, KCNB1, DCLK1, ZPLD1, AC138627.1, CACNA1E, CFAP54, DNAH11, NALCN-AS1, SNAP25-AS1, AC096719.1, DNAH6, KIRREL3, AC006148.1, ROR1, ROR2, AC010127.1, AL691420.1, ST18, LINC02343, SYNPR, CLSTN2, DENND2A, AC046195.1, PAPPA2, AC105411.1, XIRP2, AP000311.1, SHISA6, CNGB3, AC099673.1, FOXP2, ANKRD55, KCN16, LINC01470, NWD1, RGS22, CFAP61, ABCB1, CACNA1B, COL26A1, DGKG, CATSPERD, RP1, LINC01344, AC103876.1, AC073050.1, ENOX1, AC079385.1, AC091078.1, TRDN, ANO3, ANO2, ANO4, AC087564.1, FAM178B, AL031599.1, AC068051.1, GRIN2A, OPRM1, EYA1, PCSK2, RNF212B, NMNAT2, CCDC3, COL21A1, DNAH7, SLC7A13, CYP46A1, RUNDC3B, LINC00603, PEBP4, FAMN1, CHODL, AC007100.1, DKK2, SLC24A2, LINC00882, SLC24A3, AK7, TRPC6, WDR64, ZMAT4, ZFHX4-AS1, ADAMTS6, FAM19A2, AL355838.1, DOK5, AC092378.1, CNTN6, LDB2, GRM1, SPOCK3, OTOA, SLIT2, MIR548A1HG, STK32B, ANGPT1, LINC01192, PRICKLE1, GALNT3, ABCB5, IFNG-AS1, LINC01663, AC093459.1, AC117473.1, ADAMTS9-AS2, AC002429.2, CACNG2, AC125613.1, PCDH11X, EPHA5, AF121898.1, PDE1C, SV2C, AC096577.1, NPSR1, CNTNAP3B, PID1, LINC00393, LINC01692, RARB, GAS7, CHST9, AC008591.1, DOCK2, APBB11P, AC078845.1, LINC02147, FER1L6, AC002070.1, CLMP, 7SPAN8, DPF3, SCN1A, SLC35F4, ATP2B2, AC092957.1, VAT1L, DIO2-AS1, MYH11, CRB1, TBXAS1, ADAMTS18, LINC02107, LINC01060, MYOM1, AC099753.1, LINC01630, FRMPD4, AC004784.1, AC020637.1, NTN1, MLIP, AC104574.2, AL138733.1, GUCY1A2, AC093655.1, AC113137.1, FYB1, NECAB1, DCDC2, AC090403.1, KCNIP1, ATP6V0A4, TSHR, THSD7A, DRAIC, DNER, MEGF11, AL035078.4, AC011247.1, LMNTD1, AL356108.1, ACSS3, PKD1L3, SNRPN, AC021351.1, ZAN, C2orf88, TPTE, AOAH, ZNF804A, DSCAML1, COBL, FLT3, AADACL2-AS1, AC074327.1, GRAMD1B, PPP1R42, NTNG1, AC009632.2, FBN2, CLNK, SVOP, RBFOX3, AC138123.1, BACH2, UPP2, DNAH2, RERGL, PGM5, AC025159.1, ADCY8, WDR63, XACT, AGMO, CPED1, HECW2, ANK1, BMPR1B, HDAC2-AS2, NTRK3, SAMD3, LINC01411, SLC2A14, CAP2, FAM227B, LINC01170, ESRRB, PECAM1, CFAP70, AC022387.1, EBF1, AC019330.1, AC020687.1, CFAP77, RTN1, DIRC3, FLT1, SVOPL, GRIA1, TMIGD3, NOL4, SLC5A11, LINC00862, LINC01482, PCDH11Y, PDE3A, PIP5K1B, LINC00504, LAMA1, AC007563.2, CAV2, LINC01643, GPC3, PCA3, IQGAP2, AL162718.1, ASTN1, AC098829.1, AC010997.3, STXBP5-AS1, AC092691.1, RFX4, N DNAH17, MRPS22, SNTG2, AC024901.1, PLCXD3, BASPI-AS1, MROH8, RCAN2, AC016687.3, AXDND1, LINC00607, AC098864.1, LINC01588,e SLC25A21, ALDH1A1, KCNN3, FILIP1, ANKRD7, LINC02223, RASGRF2, ST8SIA1, PREX2, RBM20, ADAMTS12, DYSF, CCDC192, SLC22A10, CNTNAP4, CSGALNACT1, SPON1, PRKCQ, AC025887.2, CDH20, SPEF2, IQCM, LINC01091, EFR3B, AC073320.1, COLEC12, LINC01924, ITGAL, ARMH4, NR3C2, MYLK, TEX14, NEB, AC108866.1, GLT1D1, LINC01476, SVEP1, CCDC73, FAM171A1, NR2F2-AS1, MAPT, EML1, CPXM2, CPVL, AK8, ADAMS19, AC109927.1, PLPPR1, CPAMD8, MAP2, ITGA9, AL355076.2, SLC35F1, TMEM232, SULF1, TMEM64, GCNT2, PPP5D1, KCNK13, C12orf40, RORB, CELF4, LINC02307, OSBPL6, CCBE1, SGSM1, RNF103-CHMP3, SRRM3, GNG4, CHRM5, SLC35F2, DPYD-AS1, MMP26, UNC13A, LRFN5, PRTG, NIM1K, AC037486.1, MSRB3, AL591368.1, CHN1, GXYLT2, FBXL13, NEK10, ZFHX4, SFMA3E, CAMK4, OLFM2, TMEM192, AL117339.5, C12orf56, TEX11, ABLIM2, LINC01301, TMEM266, IL12RB2, SLC26A5, DMGDH, COL22A1, ANKRD30BL, RFLNA, CCDC102B, ITGAE, SEZ6L, ACACB, ACSM3, PCBP3, SOBP, MYO1F, MME, PKNOX2, LHFPL6, CCDC60, CPM, FBXO16, RIPOR3, LINC02438, ITGBL1, GULP1, IGHGP, PAPPA, AC106869.1, RMI2, MEF2C, OLMALINC, DCLK2, GLIDR, ZNF365, FAM81A, AL033530.1, VWF, OSMR-AS1, GRK4, C12orf42, Cl6orf95, CPPED1, PDGFD, DPH6-DT, ZNF658, RASGRP3, C12orf75, PIK3CD, CNTN3, FAM184B, HNF4G, FAM184A, EFCAB5, AC011092.2, AC027018.1.

### Gene Set 2

TCIRG1, UNC93B1, HNRNPL, ORAOV1, PTP4A2, SLC2A1, SYNCRIP, NPIPB5, OFD1, SREBF1, EIF5, BCL6, AKAPI7A, CSAD, FOSB, TCIM, WEE1, CYP4F12, KDM3A, ANXA1, PPP1R10, HIP1R, CCN72, BTBD3, IF144, MAP3K8, SH3YL1, CLK1, ULK1, STARD3, SYT1, CSNK1D, GRHL3, CYP3A5, MAOA, OSBPL2, EPHA2, TMEM259, ZFP36, AC106798.1, TRABD, UVSSA, MRPS6, PPP1CB, CEP95, UBE21, LTN1, TIAL1, RHOT2, C1orf159, FAM118A, NECTIN4, USP9Y, TMEM184A, CDK5RAP3, WASHC4, SFMA6A, APPL2, ZXDC, NECTIN1, YTHDC2, C3orf52, MTMR1, ZNF440, DAZAP1, TRIM38, DGKA, SRSF6, DMTF1, SUPT20H, COL7A1, CSNKIG2, SF1, MTX2, D2HGDH, GABPB1-AS1, ZNF326, PCF11, RAPGEFL1, ZDHHC3, MAP3K7, RBBP6, SHROOM1, KRT16, GOLGA3, PDCD6, RAB12, AC006978.2, CHMP4B, ENGASE, GBP2, PARD6B, WASL, RFC1, SIN3B, KIAA1522, HNRNPH3, LBR, SLCO9A2, MGAT1, FBXL4, PLSCR1, SELENOO, CAPN1, GLUD1, CAPN7, RAB5A, ACADVL, NPTN, GPAT4, SH2D4A, RCC2, ARHGAP27, EPS8L2, DCUN1D4, CBX3, AC009271.1, ANKMY1, TOR1AIP2, NPM1, ELOVL5, CTPS1, SPTSSB, ASPM.

### Gene Set 3

FP671120.1, FP236383.1, COL7A, SFN, AC092683.1, AHNAK, CD44, SORCS2, PGGHG, PMEPA1, ANXA1, S100A2, JAG1, MET, DSG3, OSMR, ANKRD36, KRT6A, AHNAK2, FLNA, XDH, AKR1C2, TNNI2, MTRNR2L8, CLIP4, SULF2, AC245060.5, PYGB, SSFA2, TYMP, DSC2, H1F0, ABCA7, KRT15, HMGA2, MYEOV, TFP1, CD109, S100A8, KRT5, CDC25B, SAMD9, FXYD5, SAMD9, CTSC, CNTNAP3.

### Gene Set 4

AC104041.1, KCNMB2-AS1, SMC4, ARID1B, SCMH1, WWOX, AC009271.1, CEP192, CCDC14, MIR4713HG, AC106798.1, LINC01748, SLCO3A1, TRA2B, GNGT1, WAC, LINC01572, FUS, BCL2, LINC02428, AC016205.1, NAP1L1, CENPF, EZH2, ASPM, PTBP2, FANCA, SSBP3, KAT6A, REV3L, HELLS, DANT2, ALCAM, SMAP2, TOP2A, ECT2, KCNB2, AKT3, FANC1, SCLT1, CTPS1, NFIB, TARBP1, C1QTNF3-AMACR, AC116049.2, LBR, CENPK, NEDD1, AC091057.6, L3MBTL4, TMPO, IGSF1, NFYC, RLF, SYT1, RAB12, ELOVL5, LINC01876, AP3M2, CD47, FOX13, RFC3, MKI67, MMS22L, NEO1, TRIT1, SMC6, Z94721.1, AL117329.1, GABPB1-AS1, CENPE, STK33, TCF4, KIF20B, DDX11, PAM, PRKD3, GEN1, RORA, AC092683.1, ANKRD6, NUF2, DPYSL3, ZEB1, CIP2A, IGSF9, POLQ, NCAPG2, CCDC18, SLF1, LYPLAL1, LINC00491, AC022031.2, CMC2, TTF2, NCAPG, C21orf58, ANKRD36, CIT, AC073529.1, TRMT11, AC006206.2, OTULIN, YBX1, NMNAT3, CCNF, SLFNLI-AS1, SMC2, ERO1B, CADM1, VRK1, PP1H, CACNA2D1, AC009262.1, TBCID4, LMNB1, GRBI0, BCL11A, MYB, KIF11, MYEF2, LDLRAD3, SFMA6D, CA8, LINC01456, PEX5L, NUSAP1, AC021504.1, PDE5A, NRCAM, C22orf34, CENP1, KCNMA1, TPX2, NCAPD2, LEF1, GOLIM4, VDAC3, NCAPD3, ADARB1, ANLN, KIF15, GTSE1, KIF18B, NEMP1, SGCE, TIK, TOX, TSPEAR, BUB1B, VASH2, PSIP1, CDC7, MAP1B, DLX6-AS1, PARPBP, ETV5, DEPDC1B, PLEKHG4B, NT5DC3, MYO3A, SLFNL1, USP1, ZMPSTE24, KIF23, ZNF519, SLCO6A1, SSTR2, KIF14, TGFA, ENC1, E2F7, AC106799.2, FANCB, WDR90, B4GALNT4, KIF22, RGS16, TNFAIP3, CHTF18, ORC6, ANKRD36B, SASS6, AC098850.3, RADM1AP1, ARHGAPIIA, CPE, NDC80, AURKB, NPL, CDK1, TMEM108, PROX1, TACC3, BUB1, LRIG1, LINC00958, AC019183.1, CHST15, KHDRBS3, IQGAP3, ZEB2, PGP, RAD54L, XIST, RECQL4, SLITRK6, CENP1, SYNPO2, KIF18A, JAM3, PARM1, SPDL1, SIM1, CENPO, COL18A1, OCA2, XRCC2, NBPF15, LINC02384, SLC1A4, CDON, CENPU, DEPDC1, PAK3, RFC4, CKAP2, AL603839.3, AP1S2, AC008109.1, PAPSS2, SLFN11, SGO1, KATNAL1, AL023755.1, PPT1, CCND2, NEURL1B, PHF21B, KIFC1, PRRX1, GPR173, AP001021.2, DACH1, ANKRD33B, TMEM176B, CDCA8, NPNT, STOX1, HFS6, MICU3, PTP4A3,w MEGF10, SEC11C, MIR548XHG, P3H1, MNS1, FAM161A, VWDE, CD83, DLGAP5, ZWINT, SNAP25, ZNF684, ZBTB18, ANOSL, AC106795.3, SLC38A11, DTYMK, CEP41, NKX2-2, UCHL1, SOX2, AL354994.1, THY1, FAM83D, MS4A8, HEPACAM2, NCAM1, RIMKLA, KIF1A, KIF19, ADARB2, ASCL1, NR2F1, RBM38, AC093151.3, SCN3A, S78SIA5, CERKL, PTT1G, CHGA, BCHE, LINC01811, INSM1, TFF3, CASC17, TMEM176A.

### Gene Set 5

CCSER1, PPARG, MECOM, ACER2, HPGD, DAPK1, CD96, NEAT1, AC087857.1, SNX31, RALGAPA2, BCAS1, PABPC1, LIMCH1, IKZF2, RBM47, AC009478.1, SCHLAP1, POF1B, CNGA1, SIDT1, THRB, SAMD12, PSCA, CMYA5, GATA3, CHKA, TNFRSF21, ABCD3, BICDL2, ELF3, MAML2, AC026167.1, RBPMS, ACOXL, SPTSSB, ICA1, PLPP1, ACOX1, MLPH, EPB41L1, GCLC, TBCID1, SLC20A1, ACSF2, EZR, ZNF254, NIPAL1, AC044810.3, GRAMD2B, SYTL2, SHROOM1, CD55, SPAG1, PPFIBP2, DAP, EHF, TMPRSS2, KCN115, ADGRF1, GPR39, C4orf19, SLC44A3, ST3GAL5, SLC37A1, DOCK8, ZNF440, ALOX5, TBX2, SCCPDH, PKHD1, ENGASE, FU79, LIPH, TMEM45B, ACSL5, WWC1, SWAP70, RALBP1, VGLL3, SPTLC3, ABLIM3, RHEX, SNCG, TMEM184A, GNA14, RARRES1, SLC19A2, ALAS1, NECTIN4, ZNF737, MAP3K8, PUN5, SPINK1, NTN4, GPR160, BHMT, MAN1A1, GATA2-AS1, CYP4F8, VSIG2, SCUBE2, ASS1, ZNF69, UPK1A, PTGR1, IDH1, POU5F1, RHOU, CGN, BNIPL, ADGRG6, ZNF439, DENND2D, SLC44A4, GPR78, ARRB1, CYP4Z1, GSDMB, CAPN13, POGK, ZNF761, ENTPD3, STEAP2, UPK3A, UPK1B, SCIN, MAN1C1, SLC22A5, SCNN1G, TRIM11, HMGCS2, HNMT, GATA2, ZNF486, ZNF350-AS1, CAPN5, UCA1, SLC16A5, JRK, NR1H4, PNPLA2, AC010487.3, PTCHD1, LINC01764, RAB11FIP2, FREM2, GSTM3, LINC01768, OVCH2, ALDH3B2, STS, SH3TC2, AC011503.1, CRH, MOCS1, HDHD3, CRACR2B, HLA-DRB1, PABPC3, AC027117.2, TRIM17, ZNF726, RNF207, VGLL1, CCDC198, BMP3, SH37C2-DT, LEAP2, BMP2, QPRT, HNF1B, HOPX, PIGR, ARHGAP40, SGK2, GSTM4, MUC4, ZSCAN31, ANXA9, PM20D1, LINC01833, CLIC6, CYP24A1, V7CN1, IQCJ-SCHIP1, AL589669.1.

### Gene Set 6

LINC00511, NEAT1, MAST4, RNF19A, VEGFA, VMP1, ZFAND3, CCNL1, TNFAIP2, KLF5, CSNK1A1, PTK2, ELF3, YWHAZ, THOC2, GRB7, RBM39, M7MR3, CMIP, SEMA4B, SMAD3, ATRX, NPEPPS, GRHL2, TOP2B, MECOM, VPS37B, CHD2, NCOA3, KTN1, ETS2, UTY, ETV6, PTPN13, PPP2R2A, SMURF1, GOLGA4, SON, TNFRSF21, KANSL1, NKTR, LINC00278, CD46, ERRFI1, RALGAPA2, ZFC3H1, SNX31, WSB1, TBX3, SLC14A1, ANKRD11, EZR, TCIRG1, TMEM51, TMPRSS4, KMT2E, NDRG1, SLC38A2, ZBTB7C, SLK, MID1, PPARG, ERBB2, ACTN4, SCHLAP1, SRSF11, KRT7, BRD4, ZMYM2, SRRM2, SERINC5, KDM6A, SEMA3C, PUM1, TMEM165, CCNL2, GATA3, LYPD6B, WDR45B, UBE3A, MARK3, ZSWIM6, TMEM117, UNC93B1, RNF149, EWSR1, CDH1, DYRK1A, USP3, HS6ST2, PTPRF, ADNP, TCF25, ZMYND8, KLF3, FOS, GOLGA8A, ATP8B1, ID1, OGT, PNISR, RBM5, CLIP1, PSME4, EHF, ANKS1A, ADAM10, SLC2A1, MAP4K3, RAB10, NT5C2, AC013394.1, CCND1, PPP4R1, UBE2H, TTC3, GOLGB1, RAB11FIP1, CDYL, PARP14, STK24, SETD5, GRHL1, BLCAP, FAT1, LCOR, OXSR1, NHS, KRT19, BHLHE40, KRT13, FOSL2, YBX3, DHRS3, PAXBP1, EEA1, SLTM, LINC01876, SREBF2, NAA25, USP9X, BCLAF1, KRT17, DDX3X, SERINC2, DHRS2, GNL3, LARP4B, EP300, GSP T1, SRPK1, CYP3A5, XRN2, TOP1, TCERG1, DICER1, SEMA3F, EXOC1, NAMPT, RREB1, SYTL1, CASZ1, KDM5B, ZNF207, ZRANB2, ANKRD46, ABHD17C, THRB, FBXW11, STARD3, USP9Y, RAI1, MORF4L2, FOSB, SLC23A2, DNAJC5, UBA6, BICDL2, ATP2B1, TIMM23B, HIVEP2, PRPF4B, PLEC, AP001207.3, PITPNB, SDC1, ARPC2, ORAOV1, ITGA6, RASEF, CYP4F12, PPARD, HNRNPR, PLXNB2, LMNA, FOXO1, YTHDC1, SREBF1, WASF2, ANKLE2, RCOR1, TCIM, MBD2, WEE1, PDXK, ITGB4, NLGN4Y, SRSF5, GAK, ODF2L, JUP, TSPAN14, RSRC2, IGFBP3, MAP3K8, TRAF4, DDX3Y, SUN1, CD9, RNMT, EIF5, TPM4, SECISBP2, LAMA5, ULK1, ZNF706, KDM1A, AHR, CTTN, PTP4A2, RXRA, NRIP1, NECTIN1, MAOA, AKAP17A, PDLIM1, HIP1R, TNK2, ZFP36, GRHL3, TRIM31, PRPF38B, MYO5B, KDM3A, ACAP3, TUFT1, OSBPL2, TRABD, PPP2R2D, RAPGEFL1, APOL1, ACSL1, ANX41, CLK1, HNRNPL, POF1B, BCL6, PTK6, TMC7, EPHA2, ADGRF1, RRBP1, SYNCRIP, UGCG, ASCC2, LINC01285, LPCAT4, SEMA6A, LDLR, ALDH1A3, GPRC5A, NCALD, AP003469.2, DGKA, PADI3, PPP6R1, TMEM154, KDM7A, MX2, PPP1R10, HES1, FAM129B, BTBD16, CA12, TNFRSF1A, HK2, TMEM40, PLEKHN1, NECTIN4, FAM84A, DUSP1, AQP3, SPOCD1, FAM213A, NDUFS8, ITPKC, CIS, TMEM184A, LAMB3, NADSYN1, MRPS6, RPS4Y1, HOTAIRM1, LTBR, KRT16, EPHB6, FASN, FA2H, GPR78, TNS4, CDH26, PLAT, SSH3, AC020916.1, EMP1, CYP4B1, IFFO2, MAFK, TIPARP, HGS, SERPINE1, NSFL1C, GP1, PL42G2F, C3orf52, NIPA2, OAS1, SERPINB5, EDEM1, DUOXA1, PLEKHG6, TMEM45A, DNAJA4, CEP170B, PHLDB3, MTSS1L, PIM1, AC007952.4, AC009803.1, TNKS1BP1, ATF3, HAS3, CXor137, FBXO7, FGFR3, DDIT4, DUSP5, ELF4, SRPX2, ADNP2, RARG, KDM5D, MFSD2A, KRT20, PLEKHH3, DUOX2, TRIB1, LYPD3, CTDSP1, GADD45A, PITPNM1, TAF1C, SDCBP2, FBRS, BAIAP2, MPZL2, KDM6B, AP002807.1, FAT2, MXD1, TRIM31-AS1, CYP4F3, BZW1, HS3ST1, AIM2, PER2, AL163636.1, ESRP2, ABHD11, BHLHE41, AL354836.1, PLA2G2A, MNT, TMEM86B, SEA 7A, MBD1, PPP1R15A, DUSP6, TTTY15, ADAM8, AL929601.1, RAP2B, KRT80, HILPDA, PLCD3, SMCR5, CLCA4, CYP4X1, EPHA1, OSER1, RIPK4, TMBIM1, GALE, UCKL1, MKNK2, POFUT2, ZFY, AC231533.1, GATA3-AS1, ARMCX6, AL354733.3, LINC01889, OVOL1, FAM110C, DALRD3, ZNF598, EFNB1, KLHDC7B, SERPINB1.

### Gene Set 7

CNTNAP2, DLGAP1, RBFOX1, GPC6, ROBO1, LSAMP, CACNA2D1, PRKG1, TENM3, PTPRD, KCNMA1, CALN1, NRXN1, ANKS1B, GPC5, L3MBTL4, ZFPM2, AFF3, ERC2, SFMA6D, KCNB2, ADGRL3, NPAS3, NAV3, CELF2, PEX5L, CLSTN2, SGCZ, CACNA1A, DSCAM, ADARB2, GALNT17, PPM1E, BNC2, MIR3681HG, SLC35F3, FBXL7, HS3ST4, LINGO2, TOX, XKR4, NRCAM, PDE4B, DACH1, CSMD2, GABRG3, NELL1, TMEM108, MYO3A, KCNT2, GRIK2, LDLRAD3, RELN, LINC01122, MEGF11, DOCK10, AC120193.1, EYA1, CDH4, FGF13, ERG, STK33, MAP1B, CNTN1, GHR, COL23A1, TUF7L1, AC106799.2, SYNPO2, PAK3, CPE, NOL4, GRIN2A, NTRK2, OCA2, LINC00923, LINC02384, BMPER, SNTG2, LEF1, ADAMTS12, TSPEAR, ZNF521, VIW, ANKRD33B, CA8, S78SIA6, PAPSS2, AL662796.1, AC018697.1, M72A, PLEKHG4B, IGKC, SYN2, MEGF10, LINC02438, PRRX1, TCERG1L, WDR49, IGFBP2, ADCY8, AC124254.1, ANOS1, RUNX1T1, CHST15, SSTR2, XIST, CCND2, TUBA1A, CDH11, LINC01456, LINC00470, SIM1, KANK4, PROX1, MYEF2, CXCL13, LINC01252, LINC02456, PPP4R4, SLC1A2, VSNL1, AL139042.1, MYB, SLC38A11, UCHL1, IGHG1, CD70, THY1, ENPP6, LINC02211, TMEM176B, EMILIN1, KIF1A, AJAP1, TFF3, SPSB4, HEPACAM2, HS3ST3A1, TPO, TUBB2B, PRAME, AC090825.1, CD200, RAMP1, SOX2, 7SIX, KREMEN2, HES6, C1QA, AL138767.3, RBP1, EBF3, MAP1LC3B2, IGLC3, CXCL10, C1QB, SPOCK2, CXCR4, ASCL1, TNFRSF4, ELOVL2, GBP5, SCG3, BCL2A1, INSM1, FGF2, PCSK1N, SFRP1, TMEM176A, BGN, CHGA, NKX2-2, VSTM2L, KIF19, GLYATL2, ZIC2, SELE.

### Gene Set 8

CNTN7AP2, RBFOX1, PTPRD, DLGAP1, CTNNA3, NRXN1, GPC6, ANKS1B, ROBO2, GPC5, EYS, DPP10, DMD, CACNA1A, PCDH15, PHACTR1, NRG1, CDH12, CSMD3, CTNNA2, PTPRT, CELF2, LRRC4C, CDH18, CCDC26, GRID2, AC007402.1, ADAMTSL1, TRPM3, TMEM132D, DGKB, DCC, OPCML, AL589740.1, NELL1, RALYL, CADPS, AC109466.1, AC034114.2, KCNH7, NCAM1, ATP6V0D2, AC015522.1, LINC00581, AC073941.1, PKHD1L1, RGS13.

### Gene Set 9

NEAT1, VEGFA, RNF19A, VMP1, GRHL2, GRB7, ELF3, TCIRG1, LINC00511, SFMA4B, UNC93B1, SCHLAP1, ETS2, FOS, YWHAZ, KLF5, CCNL1, UTY, MTMR3, FOSB, PTPN13, EZR, CHD2, SRRM2, SNX31, ERBB2, TMEM17, SLC2A1, DHRS3, KRT13, NDRG1, KRT7, DHRS2, SLC14A1, ERRF11, MID1, TNFAIP2, AP001207.3, NCALD, VPS37B, BHLHE40, ZFC3H1, WDR45B, CASZ1, KRT17, LINC00278, ID1, LINC01876, AHR, LINC01285, CCND1, TMPRSS4, LYPD6B, TNFRSF21, CDH1, ANX41, ZFP36, ITGA6, FAT1, RAB11FIP1, SDC1, USP9Y, EHF, STARD3, WEE1, MAP3K8, SREBF1, ORAOV1, LDLR SFMA3F, ANKRD46, NLGN4Y, AP003469.2, PTPRQ, NDUFS8, ACSL1, KRT16, SYMT1, AQP3, CYP3A5, CD9, GRHL1, EMP1, APOL1, PPP6R1, DDX3Y, C5orf17, GRHL3, ITPKC, AKAPI7A, CYP4F12, TCIM, PTK6, DUSP1, LPCAT4, GPRC5A, EPHA2, PLAT, SERPINB5, ALDH1A3, CA12, EPHB6, IGFBP3, TMEM45A, FGFR3, AC007952.4, AC020916.1, IFFO2, TRIM31, KDM5D, FAM84A, ATF3, CEP170B, AL929601.1, DUOXA1, KRT20, PER2, BAIAP2, MFSD2A, TSC22D3, AP002807.1, DUOX2, ABHD11, LYPD3, CLCA4, TMEM86B, CYP4F3, AC231533.1, HAS3, ERN2, TBL1Y, AL163636.1, AL354836.1, CYP24A1, LINC01889, EPHA1, LINC01297, PLA2G2A, TTTY15, KLHDC7B, HILPDA, ADAMTS1, OVOL1, EDN2, ANKRD37, CCDC9B, CLEC2A, HBEGF, LINC02432, AP001574.1, LINC01087, AC019349.1, PITX2, AP000527.1, SIK1, AP003469.4, TUBA3E, ADSSL1, DUOXA2, RNF39.

### Gene Set 10

MECOM, PPARG, NEAT1, PTK2, SNX31, RALGAPA2, LINC00511, GRHL2, GATA3, KLF5, SCHLAP1, TBX3, COP1, NCOA3, CD96, MAST4, ATP8B1, LINC00278, TNFRSF21, NHS, TNFAIP2, SFMA5A, SMAD3, DGKH, LCOR, ZBTB7C, 9-Sep, THRB, CD46, M7MR3, EHF, LYPD6B, TOP2B, ELF3, SEMA3C, NKTR, TMPRSS4, UTY, ANKS1A, KTN1, HS6ST2, DAPK1, RNF128, NT5C2, PTPN13, SORL1, BLCAP, SERINC5, THOC2, IKZF2, SIDT1, ACER2, RBPMS, VEGFA, TMEM5M, KRT7, POF1B, MAOA, SLC44A3, NPAS2, CCNL1, SLC14A1, TMC7, ID1, ARHGEF10L, BCAS1, SLC23A2, RAI1, GALNT1, EPB41L1, CROT, PLPP1, VPS37B, ABCD3, AP001207.3, AHR, ERBB2, AC087857.1, KLF3, DHRS2, BICDL2, PPFIBP2, RASEF, NRIP1, CAMK2G, ST3GAL1, GRB7, C16orf74, MIR29B2CHG, RNF19A, RXRA, SFMA4B, CYP4F12, ABCC3, ITGB4, CHKA, SEMA3F, PADI3, BTBD3, SYTL1, ADGRF1, AC009478.1, TMEM154, HPGD, NLGN4Y, ANKRD46, ORAOV1, RAPGEFL1, CDH26, BTBD16, KCN115, NADSYN1, CYP4B1, UNC93B1, USP9Y, PTP4A2, TCIRG1, NECTIN4, AC019117.1, SSH3, AC009803.1, PLA2G2F, GATA2-AS1, TNS4, CXorf57, ZNF552, GPR78, MTSS1L, DUOXA1, HMGCS2, DDX3Y, ABO, MPZL2, SMAD6, UGT1A8, TRIM31-AS1, AL163636.1, SMCR5, KDM5D, AC114812.2, AP001628.1, GATA3-AS1, ZFY, PLA2G2A, TTTY15, TRNP1, LINC01764, AC231533.1, LINC01768, AL131280.1, AC026369.1, CYP4F26P, EPS8L3, RAI1-AS1, AC108134.1, AC091544.5, NNAT, CHAD, U2AF1, AL033384.2.

### Gene Set 11

PPARG, RALGAPA2, ACER2, RBM47, MECOM, NEAT1, CD96, TANC2, DAPK1, TNFRSF21, NT5C2, ELF3, GATA3, SNX31, THRB, POF1B, IKZF2, AC009478.1, EPB41L1, PDE10A, CNGA1, EZR, INPP4B, RALGPS2, HPGD, ABCD3, AC087857.1, SCHLAP1, RBPMS, PPFIBP2, TMPRSS2, RASEF, ACOXL, ADGRF1, PLCE1, TBCID1, CDKL5, BICDL2, GRHL3, ACOX1, NIPAL1, ZNF254, ICA1, EHF, BCAS1, TMEM45B, ACSF2, RALBP1, ACSL5, SCAP, SPTSSB, FHL2, CMYA5, NECTIN4, VEGFA, NTN4, SPTLC3, DOCK8, SHROOM1, TBX2, TMEM1184A, RARRES1, MLPH, SYTL2, AC019117.1, RAB11FIP1, CD55, CYP4F8, CGN, KCN115, BHMT, ASS1, B4GALT1, TMEM163, VGLL3, PARD6B, ZNF737, MAN1C1, AC044810.3, PLIN5, GNA14, ALAS1, FUT9, SNCG, SLC19A2, LIPH, ABLIM3, PKHD1, PLSCR1, GPR160, SPINK1, ZNF486, CYP4Z1, OAS1, GSDMB, UPK1A, SCNN1G, ZNF69, ZNF761, LINC01768, SLC44A4, SCUBE2, ZNF66, VSIG2, UPK1B, CAPN13, NR1H4, ALDH3B2, ZNF439, CAPN5, FREM2, UPK3A, GSTM3, UGT1A8, SH3TC2, IQCJ-SCHIP1, CLIC6, CRH, HLA-DRB1, ZNF726, CCDC198, QPRT, LINC01764, OVCH2, AC011503.1, AC14812.2, PM20D1, AL031280.1, HDHD3, RNF207, UCA1, LEAP2, ZNF350-AS1, MOCS1, SH37C2-DT, VGLL1, TRIM17, BMP3, PTCHD1, AC025048.4, SEMA7A, PIGR, PALM3, ANXA9, ARHGAP40, SLC9A4, C1orf116, ZSCAN31, EPS8L3, GCKR, TERT, VTCN1, AC022034.2, TMEM238L, MUM1L1, NPIPB13, PLEKHF1, CYB5R2, PEX11A, AC099482.1, ERP27, FLRT3, AC010329.1, COLCA1, EMX2, AC108134.1, AC128688.2, AC104825.1, CHAD, LINC00840, AC005324.3, SULT1E1, CWH43, AL590999.1, LINC01336, GRM6, IL9R, CDKN2B, AC092042.3, ARL14, AL161669.3, AL354793.1, UPK1A-AS1, AP005432.2, RRS1-AS1, AC111000.4, PDZD3, AC108941.2, AL713999.1, AC000032.1.

### Gene Set 12

DPP10, MARCH1, DSCAM, MALRD1, TENM2, MAGI2, TENM3, LUZP2, LSAMP, MCTP1, CACNA1A, LRRC4C, CACNA2D1, LRP1B, CADM2, CADPS, FP236383.1, CALN1, LINC02240, LRRTM4, MDGA2, CSMD1, MGAT4C, GRID2, NELL1, UNC5D, ASIC2, MYO16, CTNND2, CTNNA3, CTNNA2, ATP8A2, XKR4, MTRNR2L8, AUTS2, MTRNR2L12, B2M, FMN2, CSMD3, CSMD2, MIR4300HG, MIR3681HG, LINC02055, LINC01317, CASC15, LINC01090, IL1RAPL1, HYDIN, GALNTL6, GAPDH, HPSE2, HECW1, HDAC9, GPC5, CLMP, CLSTN2, HBA2, GRM8, CNBD1, GRM7, GRIN2B, CNTN4, CNTN5, CNTNAP2, CNTNAP5, TRPM3, NPAS3, TMSB4X, CFAP299, THSD7B, LINC00486, LINC00276, CREB5, CCDC26, LHFPL3, FP700111.1, LDB2, CPNE4, TMEM132C, TMEM132D, FSTL5, KCNMA1, CDH12, CDH13, CDH18, KCNIP4, FTH1, FTL, GABRG3, NRG1, GPC6, PPM1E, RGS7, ELMO1, RBFOX1, RALYL, PTPRT, STEAP1B, EMCN, PTPRD, EPHA6, PTCHD1-AS, ERC2, ADAM7SL1, DHFR, NRG3, STXBP5L, ADGRL2, ADGRL3, ROBO2, RYR2, RYR3, AC113414.1, AC011287.1, DLGAP1, AC016766.1, SGCZ, SGCD, AC008415.1, AC024230.1, AC007402.1, AF279873.3, AC026316.5, AC034114.2, SLC30A10, AC068633.1, S100A4, AC099520.1, S100A11, AC105402.3, AC109466.1, DLG2, DLGAP2, ZNF385D, AFF3, PDZD2, ANKS1B, DCC, FAU, DNAH3, PCDH9, PCDH15, APOO, SNIG1, OPCML, OOEP, Z96074.1, DNAH9, ARHGAP15, NTM, NRXN3, NRXN1, SORCS1, TXNRD1, PHACTR1, EYS, AGBL1, AGBL4, SUGCT, SYN3, ERVMER61-1, UBA52, AL138720.1, AL357507.1, SOX5, AL390957.1, AL445250.1, AL589740.1, AL603840.1, DGKB, PKN2-AS1, SYT1, PIEZO2, AL391117.1, AC008691.1.

### Gene Set 13

DTNA, FHL1, GALNT17, UBE2E2, FOXP2, FN1, FAM129A, FLNC, FBXL7, FLNA, EBF1, FILIP1, TPM2, HBB, PALLD, PDLIM3, PDLIM7, PDZRN4, PGM5, PID1, SYNPO2, PLCL1, SYNM, PRKG1, TAGLN, PRUNE2, PTPRG, RBFOX3, SELENOM, SEMA3A, SETBP1, SPARCL1, SOX6, SLC8A1, SORBS1, SUTM, P1GS1, TPM1, NLGN1, NCAM1, TNS1, HSPB6, IGFBP7, TMTC1, IGA1, ITGA5, ITIH5, KCNQ5, LAMA2, LGALS1, NEGR1, LIMS2, LRFN5, MAP1B, MEF2C-AS1, MIR99AHG, MSR1, MSRB3, MYH11, MYL6, MYL9, MYLK, LMOD1, MAP3K20, SMOC2, CAVIN1, CACNA1C, ACTC1, DCN, CACNB2, ACTB, CALD1, ACTA2, CARMN, CAV1, UNC5C, CST3, CSRP1, CSGALNACT1, ZFPM2, CPED1, COL6A2, COL6A1, COL4A2, COL4A1, COL3A1, COL1A2, COL19A1, CNN1, USH2A, ANK2, CLIC4, CELF2, ARHGAP24, COL6A3, ACTG2, CHRM3, ADAMTS9-AS2, DMD, BNC2, ADGRB3.

### Gene Set 14

KIAA1671, KIF3A, ZNF654, AVL9, BAIAP2, N4BP2L2, RASSF6, KLF3, CD55, ARID5B, RAPH1, RAPGEF2, RAP1B, NAP1L4, MPPED2, MNATI1 TMC7, ZNF552, BBOX1, PIR, ZNF440, MAOA, ACOTI1, RNF38, ILIRAPL2, YWHAZ, RIOK3, RICTOR, PIM3, CFH, CEP85L, ARHGAP6, RGS12, RFX3-AS1, KCND3, MVB12B, TC2N, CDC4B, KCNQJOT1, PEL12, KCTD1, ARHGEF28, MTX2, ARHGEF3, MIR4713HG, 7M9SF2, TGFBR3, TFDP2, TBC1D4, WASF2, MCTP2, LRP4, CACNA1D, LRRC8A, MCC, MARCH6, PTPN14, TENT2, AL109930.1, LYPLA1, C1orf21, ALDH7A1, BTBD16, PTEN, NECTIN1, MEIS1, LINC02256, ZNF586, VAV3, ARIH1, LCOR, RAB6A, RAB3GAP1, USP53, LGR4, ZNF846, RAB1A, TANK, SSBP2, PWRN1, BBOX1-AS1, WSB1, PPP1R13B, AC138305.1, TBL1XR1, PPP2R2A, CASC9, PPP3CA, ARL15, NFAT5, REL, AC073332.1, CHST9, EPS8, AC009478.1, ERBB4, ERBIN, AC013394.1, ERCC1, AP003390.2, DGLUCY, ESYT2, ETS2, AP005230.1, EVAIC, UMAD1, ZFAND6, DES, DENND4C, PHC3, SH3PXD2A, FAM13B, SGPP2, SGMS1, FAM49B, AC121154.1, DHRS3, EPHA2, EPB4IL4A, ENSA, ABCC4, UBE4B, DSTN, DTNB, AP001011.1, DYRK1A, DLG5, PDLIM1, PDE7A, EFNA5, ABI1, AC019117.1, UBE2H, PEX14, ABI2, PGAP1, ABO, SLC2A1, UBE2E1, ELL2, DLEU2, DIP2B, UBE2B, ENOSF1, UBE2E3, AC023421.2, FAR1, FBXL20, SMIM35, GRAMD2B, SEC24B, CNOT2, ANKRD10, TP63, ZBTB7C, PIK3R1, CLINT1, SCAF8, HECTD2, DCAF6, TOP1, HIBCH, HIST1H2AC, HK1, HMBOX1, NUMB, CLEC2D, ATF71P2, AC104123.1, AC114812.2, CLASP1, TNFAIP8L3, SRGAP3, PICALM, TPM4, AC044810.3, AQP4-AS1, SQSTM1, FGFR2, CYSTM1, CYP19A1, AC023590.1, SESTD1, TSPAN14, FOSL2, SESN3, PAK1, CPNE8, PAFAH1B1, FRMD6, FRMD6-AS2, FRY, SEPT10, PHF21A, PHTF2, AC027097.2, TRIM31-AS1, GLI3, TAF15, YAP1. CL Gene Set 15
AKR1C2, PKHD1, ZMYM2, PLEKHA7, PLPP1, DOCK1, ZNF292, SCFD2, SREK1, SCMH1, SCUBE2, SECISBP2, SEMA5A, SENP6, SEPT9, SERINC2, SRPK2, SERINC5, SH3YL1, SIDT1, SKAP1, SPEN, SLC44A3, SLCO3A1, ABHD12, SMC5, ABCD3, SFPQ, PLXNA2, RNF19A, ACACA, POUSF1, PPARG, STMBP5, PPPIR9A, PRKCE, PRKDC, PRPF4B, STK39, ADAM10, RHEX, P7GR1, PUM2, PVT1, ST3GAL5, RABL6, RAI1, SRSF4, RBM25, RBM47, SRSF11, PTK2, ANKRD17, MECOM, ZDHHC21, COBLL1, GRIP1, HACD3, CMIP, USP25, HECTD1, TOMIL2, HNRNPC, USP3, IGF1R, IKZF2, USP31, CHD7, CHD6, INPP5A, ITGB6, CFMIP2, TMPRSS11E, CD96, KDM1A, KDM6A, KIF16B, ZFAND3, KLHL24, KMT2C, CD46, TMEM117, GNL3, TMBIM1, TRIO, COMMD10, DNM2, UBE3A, ECPAS, EDARADD, EGFR, EHF, EHMT1, EIF4B, EIF4EBP2, DLG1, UGGT2, ERICH1, UBAC2, EXOC6B, FABP5, FAM120A, FAM160A1, FAM174B, TULP4, DAPK2, FBXW11, FNBP4, TSPAN5, CRTAC1, TSIX, FRMD3, COP1, GALNT1, 7MTSF3, LHFPL2, USP47, METAP1, MFSD14C, USP34, TCERG1, MS12, XMST, MISS1, ATR, ATP13A3, TBX3, ASH1L, TBC1D1, DOPEY1, TAX1BP1, ASCC3, TANC2, ARID1B, ARHGAP32, ZDHHC20, OXR1, PABPC1, PABPC4, PACS1, PARD3, PATJ, PCF11, NEDD4L, BIRC6, MGST1, CADM1, LINC01748, TESC, CAB39L, VAPA, BRAF, MAN1A2, LYPD6B, CAPN8, BMSIP14.

### Gene Set 16

TMEM184A, SYTL2, SPIDR, TMC4, TIMM23B, TAF1D, USP9Y, ZFC3H1, TIPARP, ZNF83, SPIRE2, UTY, SRSF5, ST3GAL1, SON, ZNF638, UBC, TOB1, THOC2, WEE1, STOX2, TNNI2, VPS13C, WNT5A, TRIM31, SRPX2, WWC1, VMP1, SRRM2, VEGFA, UPK)A, TMPRSS2, TBX2, SYNE2, TTC3, TCF25, ZKSCAN1, TMEM163, ABCC3, SMCHD1, ELF3, EIF5B, EGR1, EGLN3, DUSP5, DST, EMP1, DHRS2, CYP3A5, CYP24A1, CRYBG1, CMYA5, CLMN, CLCA4, DGKH, EPS8L2, ETV6, EXOC1, HERC2, GTF2I, GPRC5A, GOLGB1, GOLGA8A, GOLGA4, GCC2, GATA3, GAK, FP671120.1, FOSB, FOS, FAT1, FARP1, EZR, CHD9, CD2AP, CCSER1, CCNL2, ANXA1, ANKRD36C, ANKRD12, ANKRD11, ALS2CL, ALOX5, AKAP9, AKAP13, AFDN, ADGRG6, ADGRF1, ACOXL, ACER2, ACADVL, ABLIM3, ARAP2, HES1, ARFGEF1, ASPH, CCNL1, CCAR1, CBLC, CARD11, C5orf17, C1orf159, BPTF, BICDL2, BHLHE40, BDP1, BCAS1, BAIAP2L1, ATRX, ATP8B1, ATF3, ARGLU1, HSP90AA1, HSPA1A, HSPA1B, RAB11FIP1, RAB10, PTPRM, PRRC2C, PRKCA, PPFIBP2, POF1B, PNISR, PLIN5, PLEKHN1, PLEC, PLCE1, PKN2, PDE10A, PADI3, RALGPS2, OFD1, RASEF, RBM6, SMAD3, SLTM, SLK, SLC38A2, SLC27A6, SLC26A3, SLC20A1, SLC14A1, SHANK2, SETX, SCHLAP1, SAMD12, S100P, RNF213, RIMS2, RASGEF1B, SMG1, NR4A1, NKTR, KTN1, KRT7, KRT19, KRT18, KRT17, KRT13, KMT2E, KLF5, KIFC2, KIAA1109, JMJD1C, INSR, INPP4B, INO80D, HSPH1, LENG8, NPAS2, LINC00278, LINGO1, NEAT1, NDRG1, NCOR1, NBEAL2, MYOF, MYO6, MUC20-OT1, MLPH, MAST4, MALAT1, MAGI1, LUC7L3, LRRFIP1, IMO7, LMNA, LINC00511, ZNF91.

### Gene Set 17

GALNT17, CCDC26, LUZP2, TENM2, LSAMP, LRRTM4, LRRC7, LRRC4C, LRP1B, GALNTL6, TRPM3, MAGI2, LINC02511, CDH12, CDH13, CDH18, LINC02240, LINC02237, LINC01830, SEL1L2, LINC01435, LINC01358, LINC01317, LINC02267, LINC01090, EPHA6, SLC4A10, MIR3681HG, SNTG1, RNF219-AS1, MGAT4C, ERVMER61-1, VEPH1, DSCAM, RPH3A, MDGA2, HPSE2, MALRD1, RYR1, C8orf37-AS1, CA10, CACNA1A, USH2A, CACNB2, CADM2, CALN1, RYR2, UNC5D, RYR3, C8orf34, MIR4300HG, HYDIN, TNR, SGCZ, CUBN, CUX2, KIF6, KHDRBS2, DCC, KCNQ5, TMEM132D, DGKB, TMEM132C, CTNND2, KCNIP4, KCNAB1, DLGAP2, DMD, DNAH3, DNAH8, DNAH9, SLC14A2, DOK6, DPP10, DPP6, DLG2, CFAP299, CTNNA3, SGCD, LINC00536, LINC00535, LINC00534, SEMA3A, LINC00276, CNBD1, CNTN4, CNTN5, CNTNAP2, IL12A-AS1, CTNNA2, CNTNAP5, COL24A1, COL25A1, ILIRAPL1, EGFLAM, THSD7B, CPNE4, CREB5, CSMD1, CSMD3, KLHL1, COL14A1, SORCS1, ROBO2, ADCY2, ZNF385D, SYN3, GRID2, ZNF804B, FMN2, AC008892.1, AC008691.1, AC008415.1, AC008050.1, PDE1A, ADAMISL1, ADAMTSL3, ATP6V0D2, PCDH9, ZFPM2, PCDH15, AFF3, AGBL1, GRIN2B, GRM5, GRM7, GREB1L, PHACTR1, PTPRT, PKN2-AS1, AC018742.1, PTPRD, AC026316.5, AC034114.2, AC068631.1, AC068633.1, AC012409.2, AC090568.2, AC092100.1, AC092422.1, GRM8, AC092650.1, FP700111.1, AC010343.3, PLDS, AC109466.1, AC009975.1, AC113414.1, PPFIA2, SUGCT, GPC5, SPTBN4, PLXNA4, AC016766.1, OPCML, RALYL, NEGR1, AP003181.1, RGS7, AQP4-AS1, RIMS1, RIPOR2, NCAM2, RIT2, NALCN, NUP210L, MYT1L, KR4, FAM19A1, HCN1, MYO16, ASIC2, HECW1, PRKG1, TARID, EYS, MYO3B, SOX5, SORCS3, ANKS1B, AL138720.1, AL158198.1, AL391117.1, AL445250.1, AL445584.2, AL589740.1, AL603840.1, AC007402.1, GSG1L, NKAIN3, ALK, RBFOX1, NRXN1, NRG3, NRG1, SPATA16, NOS1, NLGN1, NTM, FSTL5.

### Gene Set 18

ITGA2, GLIS3, GIGYF1, ISG15, INTS1, ST5, SLC25A37, HIF1A, SSFA2, SORCS2, HLA-DRA, HMGA2, SLC9A7, H1F0, HSP90AA1, HSPA1B, INO80D, HSPB1, SLC27A6, IFI27, SLC26A3, IGF2BP2, IL4R, INF2, INHBA, SLC30A10, ITGA3, ABCA1, ITGB6, MTRNR2L12, MTRNR2L8, MISS1, MYEOV, MYO18A, MYO1B, RGS20, NET1, NOL8, RBM25, RASGEF1B, OSMR, PADI1, RAB31, PARP10, PYGL, PYGB, PVT1, PGGHG, PKP3, PLA2R1, PLAU, PLEC, PMEPA1, PTPN6, PTPN18, PPP2R3A, PTGR1, PRSS3, MKI67, ITGB4, MIR4435-2HG, MIB1, ITGB8, JAG1, JAK2, SLC16A3, SLC12A7, KIFC2, SFN, KNOP1, KRT3, KRT15, KRT16, KRT5, KRT6A, KRT6B, SERPINE1, KYNU, LAAMB3, LAMC2, LINC00854, SAT1, SAMD9L, SAMD9, MACC1, S100A8, S100A2, RRBP1, MBOAT2, RPL35, MET, MIR222HG, LINGO1, PRR16, FYB1, DDX58, AP002495.2, ANXA2, ANXA1, DNAJB1, ANO9, DPYD, ANKRD36C, DSC2, DSC3, DSG3, DSP, UBC, TKT, ANKRD36, TIMM23B, ANKRD18A, EEF1D, ZBTB7A, EGFR, EHBP1, AIS2CL, EHBP1L1, THSD4, AKR1C2, ARFGAP1, CTSC, ARHGAP29, TMPRSS11E, CCDC88B, CD109, UBASH3B, CD44, TYMP, GABRE, CDC25B, UHRF2, CDK12, CDK6, BRD9, CES1, ZFP36L1, TN7N12, CHST11, CLDN1, BARX2, WDR34, CLIP4, ASPH, XAF1, CNTN7AP3, COL4A6, XDH, COL7A1, ARHGEF4, TNFSF10, EML3, ANKRD36B, SYNE2, FAM50A, FAM111A, FP236383.1, AC121154.1, FMN1, AC245060.5, FAM83A, TBCID2, AC103718.1, SULF2, FAM3C, TAF1D, FRMD6, ACTN1, ABCC3, FXYD5, STAT2, SYT8, AC092683.1, ABCA7, EPS8L2, TFP1, AC245041.2, SVIL, AHNAK, AHNAK2.

### Gene Set 19

AC026167.1, WDFY3, AC087857.1, MLPH, ASH1L, BCAS3, WWC1, ATP11B, BCAS1, RABGAP1L, BICDL2, PRKCE, ZNF91, PRKCA, PSCA, MAGI1, UGGT2, MAML2, ABCD3, POF1B, PPP1R12B, MAP3K5, MAP7, MAPK10, C4orf19, BTBD9, ABHD12, AC009478.1, ZNF644, CAMKMT, PPFIBP2, N4BP2L2, PLEKHA5, ACOXL, ACSF2, ZNF254, QK1, ADGRF1, AFF1, ANKRD12, ACOX1, PBX1, PAN3, ZDHHC20, AHR, RALGPS2, PACS1, OXR1, RAD51B, PATJ, PTPRM, PKN2, ACER2, RNF128, MYOF, XPR1, NAALADL2, ARGLU1, PLCE1, YAP1, NCOA1, NCOA2, ARAP2, NEDD4L, RC3H1, NFE2L2, RBPMS, NIPAL1, NIPBL, ZBTB20, PLPP1, SAMD12, LRBA, CCSER1, ELF1, EHMT1, IKZF2, EFNA5, IMMP2L, INPP4B, DST, ITSN2, ICA1, SLC20A1, DOCK1, DNM2, KAZN, DLG1, KCN115, DENND1B, SIDT1, DAPK1, DOCK8, HUWE1, SLC44A3, EPB41L1, GCLC, SSH2, STK38, STOX2, GMDS-DT, GNAQ, SPTSSB, GPR39, GRAMD2B, SYTL2, FBXL17, FARP1, SPAG1, SORL1, FAF1, TANC2, TBC1D1, SMCHD1, HPGD, KIAA1217, DAP, TMCC1, CDKL5, SENP6, TOMIL2, TRERF1, LIMCH1, CEMOP2, CMYA5, CNGA1, TRAK1, TTC2, CD2AP, CRYBG1, TMPRSS2, CHKA. CD55.

### Gene Set 20

ZMYM2, TOP2B, SMARCC1, USP3, USP34, SON, PUM1, TBCID22A, TBL1XR1, SMURF1, ZMYND8, UTY, SNX31, TBX3, TMEM51, SPIDR, STK24, UBE3A, PSME4, UBR5, TTC3, PTK2, PTPN13, SCHLAP1, ZNF638, SRSF11, SRPK2, SREBF2, TRIO, SDK1, SPPL3, UNC93B1, PTPRF, SMAD3, SRRM2, SLTM, VEGFA, RAB10, RNF19A, RNF149, SEPT9, RBM47, RBM5, RBM6, WSB1, TNFAIP2, RBM39, SERINC5, TMEM117, SLC14A), YWHAZ, SETD2, TMPRSS4, SEM5, SH3PXD2A, TMEM165, RERE, SLC23A2, UBE2H, TNFRSF21, TCF25, RAB11FIP1, TCIRG1, VMP1, SLC38A2, SFMA3C, THOC2, ZFC3H1, RALGAPA2, SEMA4B, THRB, ZFAND3, VPS37B, SEMA5A, ZBTB7C, RNF213, TIAM1, RBFOX2, WDR45B, SLK, STAG1, ZSWIM6, GMDS, CHD2, KANSL1, GATA3, ERBIN, ITCH, IGF1R, CDYL, CDH1, ID1, CD96, ERRFI1, ETS2, HS6ST2, CD46, PRRC2C, ETV6, CCNL2, CCNL1, EWSR1, EXOC6B, MECOM, EZR, FATI, BRD4, KRT7, BPTF, BLCAP, CLIP1, CMIP, COP1, ERBB2, KTN1, EHF, KMT2E, LARGE1, KMT2C, LARP4B, LCOR, LINC00278, LINC00511, EEA1, DYRK1A, EIF4G3, KLF5, BIRC6, DHRS2, LRRFIP1, LUC7L3, LYPD6B, MALAT1, MAML3, KLF3, MAP4K3, MARK3, MAST4, ELF3, CTNNA1, KDM6A, CSNK1A1, LPP, FLNB, BRAF, FNDC3B, NPAS2, NT5C2, ANKS1A, ANKRD11, OGT, PABPC1, GTF2I, GRHL2, AKAP9, PARD3, PARP14, PDE4D, PDE8A, NKTR, AKAP13, ADAM10, GRB7, ACTN4, GOLGB1, PLEKHA7, PNISR, PPARG, GOLGA8A, GOLGA4, FTX, PPP2R2A, PPP4R1, PPP6R3, ADNP, NHSL1, NPEPPS, NCOA3, FOS, ARX, MED13, ATP8B1, MED13L, ATAD2B, MID1, MSI2, MTMR3, MYO6, HERC2, ARIH1, NDRG1, NEAT1, KRT19, ARID1B, ARFGEF1, FP671120.1, ARHGAP32, NFAT5, FOXP1, NF1.

### Gene Set 21

TBCID4, FT1, GAPDH, SYT1, FUS, TCF12, FLNA, ENSA, EZH2, FOX13, TENM3, ENO1, FTL, STMN1, STK33, TAGLN2, TCF4, PRDX1, EEFIA1, CALR, CACNA2D1, BCL2, B2M, WAC, AUTS2, ASPM, WWOX, ARID2, XPOS, YBX1, APOO, AMBRA1, ALCAM, ZEB, AKT3, ZMYM4, ACIG1, ACTB, AC104041.1, AC016205.1, AC009271.1, ZNF90, CAMK1D, EEF2, CANX, CASC15, DPYSL3, DLGAP1, DEK, CTPS1, TMPO, COX4I1, COPA, TMSB10, TMSB4X, CHCHD3, CFL1, TOP2A, CENPF, TOX, CDKAL1, TRIT1, CD74, TUBA1B, TUBB, TXNRD1, UBA52, CD24, CBFA2T2, CAP1, CEP192, HNRNPU, SSBP3, RPL3, RPL3A, RPL14, RPL15, RPL18, RPL19, RPL23A, RPL27A, RPL28, RPL29, RPL3, RPL30, RPL32, RPL35A, RPL36, RPL37, RPL37A, RPL38, RPL4, RPL41, RPL5, RPL7, RPL8, RPLP0, RPLP1, RPLP2, RPSI1, RPL11, RPL10, RORA, ROBO1, PPP3CA, PTMA, PTMS, PPP1R9A, PPIA, PTPRK, PKM, PHIP, PHF14, PFN1, PEX5L, PAM, PABPC4, RPS12, RACK1, OOEP, NUF2, NUCKS1, NFYC, NFIB, NFIA, REV3L, RFC3, RFX3, NEW, NAP1L1, RLF, MIR4713HG, RAD21, GDI2, RPS14, RPS16, ITPR2, IFI16, SLC38A, HSP90B1, HSP90AB1, SLCO3A1, HNRNPK, HNRNPA2B1, HNRNPA1, SMAP2, HMGN2, SMC4, HMGN1, HMGB2, SMYD3, HLA-B, HLA-A, HIVEP3, HELLS, H3F3B, H2AFZ, H2AFY, H1FX, GSTP1, GNGT1, GNAS, SRSF3, IVNS1ABP, KAT6A, KCNB2, KCNMB2-AS1, RPS18, RPS19, RPS2, RPS20, RPS23, RPS24, RPS27, RPS27A, RPS3, RPS4X, RPS5, RPS6, RPS7, RPS15, RPS8, RPSA, MAP1B, SCAF11, LINC02428, SCMH1, LINC01748, LINC01572, SEMA6D, L3MBTL4, SERF2, SFPQ, KLF12, SIK3, RPS9, MACF1.

### Gene Set 22

CERS6, PACSIN2, PAK1, ARMH3, PAN3, ARNTL2, ARPC2, CFLAR, NUB1, ASAP1, ATAD2B, TBCID4, ATP11C, SLCO3A1, CCSER1, SLCO5A1, PFKFB3, TBC1D8, TAOK3, NFKB1, GLS, GPR157, NAA25, RAB10, GPBP1, GNPTAB, SLC41A2, DENND5A, NFAT5, TMEM131, GNB1, REL, NCOA7, NEU3, ARHGAP31, TGFBR1, NF1, NAV1, AMTP2C, TAOK1, FNBP1, PLXNC1, POGLUT1, ST3GAL6, ST3GAL5, ETV6, CCDC26, ERICH), FAM129A, PTBP3, SPRED2, PTPN2, ENOX1, RAB8B, SPPL3, SPECC1, ELMO1, PTPN1, ST8SIA4, FAM135A, FAM3A, TAB2, FLT3, PHKB, PICALM, DOCK10, SUZ12, CCN1, SMC5, PIK3CB, BASP1, PKN2, FAR1, FAM49B, FAM222B, STK4, RALA, BCL6, MYO1G, RELB, NABP1, HCK, LRRK1, SINHCAF, ITGA4, MAP2K1, MAP3Kl, MAP3K13, WNK1, MAP3K14, MAP4, COP1, DENND1B, MAPK8, ACTR3, RTN4, MBD2, ITSN2, MBOAT2, ZBTB46, JAK2, KMT2C, LAMP3, ABCC4, ZNF516, ZNF366, LCP1, KIF2A, CEP350, KDM2B, KDM2A, CELF2, ZFAND3, LPAR1, JARID2, LPP, LRRFIP2, VAC14, RUFY3, IL13RA1, ID2, SLC22A23, IL15, DAPP1, ALCAM, HPS5, HOOK3, IDO1, MIS18BP1, HLA-DRB1, HLA-DRA, HLA-DPB1, HLA-DPA1, MKL1, TRIO, MOB1B, HLA-DRB5, IFNGR1, RAB12, RNF144B, CTTNBP2NL, RNF145, CD74, AKAP3, AFDN, USP53, CSNK1A1, AFTPH, CD80, CD83, USP12.

### Gene Set 23

CHD7, CR1, SFMA6D, DNAJC10, RRAS2, RCSD1, RABEP2, SCIMP, CLSTN2, RAI14, CNR2, CXCL13, COL19A1, KRT15, RALGPS2, DRAM2, COL4A3, EBF1, DSP, MACROD2, ELOVL5, IGHD, MGAT5, IGHM, MEGF10, MEF2C-AS1, MEF2C, IGSF1, MARF1, MARCH1, MAP4K4, INPP5D, LY86-AS1, LY86, JAM3, KCNH8, LINC02422, KCNAB2-AS1, LINC02397, KHDRBS2, LINC00926, LARGE1, MICAL3, MIR3681HG, HVCN1, MIR548XHG, PRKCE, PRDM2, POU2F2, FAM117B, FAM177B, PLEKHG7, PLEKHG1, PKIG, FCMR, FCRL1, EHBP1, FCRL2, FP671120.1, PAWR, PARP15, OSBPL10, GGA2, NCF1, GNG7, GNGT1, GRAPL, MS4A1, PAX5, FSTL4, KRT16, TEX9, ST6GALNAC3, BACH2, AL662796.1, BANK1, STRBP, AP001636.3, STPG2, WASHC4, BOD1L1, BCAS4, BCL11A, ADAM28, CD37, AL355076.2, ARHGAP24, CD22, TLR10, TMEM108, TMEM131L, TMEM156, SNX25, USP6NL, ADK, AFF3, SMAP2, TNFRSF13B, AIM2, BLK, UGT8, AC119396.1, STX7, AC120193.1, CD79B, AC027097.2, ANKRD33B, C12orf42, AC022182.1, TP63, SSBP2, SFSN3, ZHX2, ATP2B1, AC008878.3, ANO9, ZNF107, CAMK1D, SETBP1, AC009271.1, SP140, AL117329.1, ZDHHC14, ZCCHC7, SHISAL2A, AC104041.1, AC106798.1, ANKRD13A, SIPA1L3, AUTS2, SYNPO2, SWAP70, AC106799.2, SSH2.

### Gene Set 24

GPR183, MX1, MX2, MTMR1, MYB, GZMB, GRAMD1B, CERS4, APP, TARBP1, TASP1, TBCID32, PARP14, TCF4, PALD1, ARID4B, P2RY6, GABRG3, P2RY14, P2RX1, ARID3A, OFD1, ARID2, NR3C1, ARID1B, HDAC9, GLT1D1, TGFBR2, TNRC6B, MPEG1, HSP90B1, MNAT1, WDFY2, INTS6, WDFY4, XAF1, IRF4, IRF7, IRF8, ITCH, MALT1, MALAT1, ZNRF2, XIST, LTB, ITPR1, ISAMP, ZDHHC17, JAML, JAZF1, AC023590.1, ZFAT, LINC01684, KDM5A, LINC01478, LINC00996, LAMPS, INPP4A, VRK2, IL3RA, ADAM19, HIVEP1, HIVEP3, HLA-A, HLA-B, TSPAN13, ANKRD12, ANKRD11, TUT4, HS3ST4, MIR4432HG, FP236383.1, UGCG, TRIM22, IFI44, IGF2BP2, IGF2R, USP24, AHI1, UTRN, MED13L, MDM4, MDFIC, ADARB2, UVRAG, MCOLN2, VASH2, IFI44L, ATP2A3, GALNT2, RABGAP1L, RBM33, DNASE1L3, DOCK2, CCND2, SMC6, DPYSL2, SMPD3, CCDC88B, PDE4B, CCDC50, SLC7A6, CCDC186, EIF2AK2, SOX4, SP110, CARD11, PTPRS, ENPP2, C22orf34, EPDR1, EPHB1, ERN1, SNX9, PRKCB, CD2AP, RFTN1, CHD9, SDK2, SCN9A, CLCN5, SCAMP5, CLEC4C, CDYL, SAMD9L, RUNX2, RUFY4, RERE, COL24A1, RUBCN, SLC12A3, c SLC15A4, SLC20A1, CUX2, CXCR4, CYFIP2, RHEX, RGS7, SLC35F3, RUBCNL, ST3GAL2, DHTKD1, PLAC8, PHC3, PI4KA, STAP1, BID, PIK3API, FMNL3, SULF2, FAM129C, STAT2, PHEX, BLNK, FCHSD2, FAM160A1, EXOC6, PDE7A, FBXW11, PMEPA1.

### Gene Set 25

SIGLEC1, SIPA1L1, NRP1, RIN2, SLC11A1, WDFY3, MAP3K8, SLC11A2, RNF13, RREB1, RNF149, MANBA, NT5C2, WSB1, SLC16A10, RNF130, PIK3R5, SLC2A3, NUMB, LYN, PMP22, SH3PXD2B, ZNF804A, ZNF710, ZNF438, ZNF331, LGMN, LHFPL2, SFMBT2, LIMS1, ZMIZ1, PDGFC, PEAK1, SGPL1, PHACTR1, TBXAS1, TANC2, SBF2, ZFYVE16, ZFHX3, SAT1, SASH1, LITAF, SAMSN1, TET2, SAMD4A, ZEB2, LPAR6, PLA2G7, SYK, LRMDA, MB21D2, VMP1, RAB20, TTYH3, RAB1A, MITF, NAIP, TMEM51, TTC7B, TNFAIP2, QKI, TRPM2, PTPRE, SPRED1, MKNK1, MYO9B, PTPN12, MOB3B, SRGN, TNS3, MYO1F, MRC1, PSAP, MS4A6A, MSR1, MYO1E, ST3GAL1, PPARD, MXD1, NAMPT, RGL1, RAB31, TYMP, NPL, PLAAUR, NFE2L2, SLC43A2, MCTP1, TFEC, RBP1, RBMS1, SLC8A1, RBM47, MEF2A, TFRC, SLCO2B1, RASSF4, TGFBI, MERTK, MFSD1, RASAL2, RAPGEF1, STARD13, NEAT1, PLSCR1, MIR181A1HG, TLR2, PLXDC2, SNX29, UBE2E2, STAB1, PDE4DIP, ZSWIM6, BACH1, CCDC88A, GAB2, GAS7, C20orf194, BMP2K, GK, BAZ2B, GLUL, AXL, GNA13, ATP8B4, GNAQ, ATP6V1B2, GPR137B, ATP6V0A1, ATP1B3, ATP13A3, IL18, ARHGAP10, ARHGAP18, ARHGAP22, ARHGAP26, ARHGEF10L, FRMD4B, HIF1A, HBEGF, AWG7, GSN, GSAP, GRK3, GRB2, ASAH1, AP2A2, FRMD4A, CD163L1, DOCK5, DMXL2, DPYD, DENND1A, EEPD1, DAPK1, ELL2, EMILIN2, KYNU, CUX1, CTSB, CTNNB1, EPB41L3, CSF2RA, EV15, CMIP, F13A1, FNIP2, CD86, FNDC3B, FMNL2, FMN1, FHIT, FPR3, FGD4, FCGR2A, CEP170, FAM49A, FAM20A, CIITA, CLEC7A, FCHO2, AP003086.2, DOCK4, ALOX5, ACSL1, ACER3, ISN1, ITPR2, ADAP2, KCNMA1, ITGAX, ADGRE2, ITGA9, IRAK3, AC074327.1, KIF1B, ABR, ADAM9, ABCA1.

### Gene Set 26

CD2, CD226, PCNX2, SLC38A1, CD3E, CD3G, PDE7B, ABHD17A, RAB27A, CD8A, SLAMF6, DAPK2, SLA2, LINC01934, LINC01871, AC243829.1, AC116366.3, SLF1, KLRC4-KLRK1, RASA2, SRGAP3, JAKMIP1, CBLB, JAKMIP2, RASGRP1, SNTB1, SLA, PAG1, KLRD1, CCL5, SLFN5, SLFN12L, DTHD1, AC243829.2, PARP8, LAG3, SKAP1, SIRPG, KCNQ5, PPPIR16B, PPP2R2B, SAMD3, RUNX3, PRF1, PRKACB, PRKCH, LCK, ZFYVE28, PRR5L, PSTPIP1, PYHIN1, PTPN22, PTPN4, PTPN7, KLRC1, RPS6KA3, RIN3, EVL, CLNK, ZBTB20, CD96, KIAA0825, PIP4K2A, SIDT1, BTN3A1, CDK6, LINC00299, SGMS1, ABCB1, CYTOR, KIAA1671, PLPP1, AC022126.1, CLEC2D, AC022075.1, LINC01358, TRAF5, PAM, HLA-C, SYTL2, ITGAL, SYTL3, GNG2, NAP1L4, GNLY, ATP8A1, IKZF3, LYST, GZMA, GTDC1, ITGA1, ITGAE, MT-CO1, APBB11P, MYO7A, GPR174, AKNA, TBC1D10C, GRAP2, TMSB4X, ARAP2, AL645568.1, MT-CO3, MT-ND4, MT-ND3, MIAT, TBCD, TRERF1, NLRC5, TUFBR3, IL18RAP, THEMIS, TIGIT, NLRC3, TRGC2, SYNE1, ADAMTS17, NFATC3, STK39, AOAH, ARHGEF1, MPHOSPH9, TNIP3, TRG-AS1, NELL2, B2M, IL2RB, ATXN1, MCTP2, NFATC2, TOX

### Gene Set 27

CSGALNACT1, TMC8, CMTM8, RORA, CRYBG1, ARHGAP15, ANKRD44, CNOT6L, S18SIA1, AC006369.1, TRAF3IP3, RBL2, ZNF831, DGKA, TRAT1, DENND2D, TNRC6C, RETREG1, TRBC1, DDX60, TNIK, TNFSF8, RHOH, CYTIP, TNFRSF25, APBA2, TSHZ2, RNF125, ABLIM1, TNFAIP3, RNF144A, AC010609.1, TNFAIP8, CEP85L, SARAF, CD44, CD247, CCSER2, CCR7, SYNE2, ADD3, ACAP1, TRANK1, CCND3, RASGRF2, SLC16A7, SMCHD1, SORL1, CATSPERB, SPOCK2, SPON1, CAMK4, STK17A, STAT5B, BTG1, BTBDI1, STAT4, BCL11B, ZFP36L2, CD6, CD69, ARHGEF3, TESPA1, TCF7, TTC39C, TC2N, SCML4, SELL, ANK3, SEMA4D, SENP7, AC139720.1, SEPT6, BICDL1, SIGIRR, CDC42SE2, CDC14A, ZC3HAV1, TXK, ATP 10A, AKT3, ATP2B4, ZAP70, SERINC5, RIPOR2, AAK1, RASA3, FAAH2, ITK, ITPKB, MLLT3, JAK3, HIVEP2, MAN1C1, IQGAP2, ETS1, IPCEF1, FOXP1, PIK3CD, KLF12, PDE3B, PPP2R5C, FAM19A1, HELB, MPP7, PITPNC1, KIAA1551, FKBP5, LINC00861, GIMAP7, NCK2, RASAL3, LINC00623, GPR155, GPR171, GPRIN3, KAT6A, GREM2, LINC01550, NR3C2, PDCD4, PRKCQ, PIK3IP1, PRKX, IL79 FYN, DOCK9, FYB1, MGAT4A, IKZF1, ODF2L, MBP, EMB, INPP4B, EML4, PTPRC, LEF1, MCUB, LEPROTL1, OXNAD1, MBNL1, EPB41, P2RY8, PCAT1, PSMA1.

### Gene Set 28

GMDS, ITGA8, OSBPL3, CASP10, GIGYF2, SPCS2, GLCCI1, ACOXL, SPA7S2, AKAP9, BCL2L11, GAS6, SSR3, SSPN, ACSS1, C11orf80, NUGGC, VOPP1, USP48, GBF1, SRP54, NXPE3, CADM1, OGT, BICD1, CARMIL1, ST6GAL1, BTG2, MYO1D, GMDS-DT, IGHG1, TMEM117, IGHG3, ARFGEF2, IGHGP, TMEM39A, TSHR, IGKC, IGLC2, IGLL5, TSC22D3, ANKRD36B, ANKRD36C, INSR, MCEE, MBTPS1, TOP1, MBNL2, TP53INP1, TPD52, TPST2, TMC3-AS1, NEDD9, IGHA1, ANKRD36, NDUFAF6, USO1, NCOA3, MAN1A1, UGGT2, SYTL1, UBE2H, UBE2G1, MZB1, MSI2, AL591518.1, TBC1D9, HERPUD1, TXNDC11, HIPK2, TENT5C, ITGA6, HM13, ANKRD28, HSH2D, THEMIS2, IFNG-AS1, SOX5, SNRNP70, LMAN1, CPEB4, COBLL1, ESR2, SAMD12, PRDM1, POU2AF1, CLPTM1L, EXOC4, PMM2, CLIC4, SCFD1, LIN52, CHST15, AC016831.7, SEC14L1, SEC24A, SEC24D, SEC31A, SEC63, SEL1L, SEL1L3, PLPP5, FAM214A, CPNE5, CHODL, CREB3L2, ESR1, RAPGEF2, DNAJC3, RASSF6, RBICC1, DNAJC1, RBM6, LARP1B, DERL3, DENND5B, LAX1, EAF2, EDEM1, RAB30, EHMT1, EIF2AK3, RHBDD1, EIF2AK4, LCORL, RIC1, ZNF215, AC008014.1, ERC1, RRBP1, KLHL6, PLCG2, SEC61A1, AC092683.1, SLC15A2, CCDC88C, PECAM1, PDK1, WWOX, LINC02384, CCPG1, SLC17A9, FUT8, PAPSS1, FOXO3, CD38, ZBP1, ABP1, LINC02362, SLC44A1, PCMTD1, KCNN3, PGM3, XRN1, FNDC3A, FAM69A, CFAP54, FBH1, FBXW7, CEP128, CDK17, PIP5K1B, PIM2, CDK14, SLAMF7, WNT5B, FCRL5, AC078883.1, AC087280.2, AC092546.1, TRAM2, SND1, GAB1.

### Gene 29

INPP5F, MARCH3, MAST4, LRBA, KMT2A, ACSL4, IL2RA, ZNRF, UBR5, LDLRAD4, KAT2B, ZEB1, MAN1A2, USP15, AC013652.1, TULP4, ZC3H7A, AF165147.1, ZC3H12D, TN, MAF, TSPAN5, UXS1, MAP3K5, JAK1, AL357793.1, LINC01572, AC008105.3, ZNF292, LINC02099, IL21R, NOP58, TRAF3, SLAMF1, PELI1, CD7, SLC12A6, FOXN3, FOXO1, CD28, PCED1B, PBXIP1, PBX4, FOXP3, SMC4, PAK2, SMYD3, CCDC7, CCDC141, GALM, GALNT10, SPATS2L, PHACTR2, CASK, PHF2JA, SH3KBP1, RAP1GDS1, RAP1A, DGKH, DUSP16, R3HDM1, PVT1, PTPRJ, ENTPD1, ENTPD1-AS1, CTLA4, RNF213, RPS6KA5, RTKN2, CRADD, F5, FAM172A, CHST11, PLCL1, FARS2, PHTF2, ILIR1, SRPK2, GATA3, ARHGEF6, ARHGEF12, HNRNPLL, HS3ST3B1, MIR4435-2HG, THADA, ICOS, TLK1, APOLD1, IKZF2, TNFRSF18, TNFRSF1B, IL12RB2, TNFRSF4, TNFRSF9, IL18R1, MCF2L2, TOX2, TPTEP2-CSNK1E, ARID5B, C15orf53, ARNTL, TBL1XR1, GBP2, NSF, BIRC3, GBP5, STAG1, STAM, BCL2, GCNT1, STIM1, STK17B, BATF, ATRX, NCOA2, NCOA1, NCALD, GOLGA8B, GPHN, TANK, ASXL1, TCAF2, L3MBTL3.

### Gene Set 30

SORBS2, NLGN1, NNMT, FBN2, DGKB, SPEG, DF, RBFOX3, RBPMS, FERMT2, PRUNE2, SORBS1, CASC15, FGF7, OSMR, PARM1, SMTN, SMOC2, DLG2, PARVA, BOC, NDE1, MYH11, ASB5, HLA-DRA, RNF217, MYOCD, HIF3A, MYOM1, SYNPO2, SYNM, SYNE1, DMD, BCL2, HDAC9, STK38L, NAV2, NBEA, BMPR1A, NEXN, LDB3, CCND2, SLMAP, CSRP1, RFHOB, SHROOM3, FOXP2, PDZRN4, PEX5L, FNBP1, SBSPON, PHLDB2, PGM5, RYR3, RYR2, RUBCNL, CPXM2, FLNC, FLNB, CPEB4, SLC22A3, FHL1, CNTN1, CNN1, GAS6, RGS16, CYR61, GALNT17, SLFNL1-AS1, GADD45B, CELF2, FHOD3, CTGF, SLC35F4, CHRM3, CKB, CLIC4, SLC35F3, PCP4, FSTL3, CLU, CSRP2, TGFBR1, MYLK, HPSE2, EGR1, PTGS2, LPP, ADAMTS9-AS2, ADAMTSL3, ADCY2, JPH2, ADAM19, ADCY5, PRDM6, MBNL1, AF165147.1, ARID5A, TSPAN2, AFF3, RAMP1, ADGRB3, KCNQ5, ACTN1, ACTG2, ENAH, LGALS9, EMILIN1, PTGIS, XIST, PRKD1, LAMA3, LINC00578, ELN, LMCD1, AC027288.3, LMOD1, WFDC1, AC131025.2, ACTA2, EOGT, ACTC1, ITGA5, TPM2, ESYT2, AKAP6, TNS1, FAM129A, AKAP12, TNIK, MEIS2, MFGE8, MINDY2, MIR99AHG, TMEM108, MSRB3, IFITM3, ID4, FAM83D, THSD4, MYB, HS3ST4, THBS1, ANKS1B, TOX, RNF150, ALKAL1, IQCJ-SCHIP1, TPD52L1, ALCAM, DTNA.

### Gene Set 31

ESM1, PLPP3, PLEKHG1, EXOC3L2, PRKCH, FLI1, ETS1, PLCB1, PLEKHA1, FGD5, PICALM, PLCB4, PITPNC1, PLD1, PLVAP, ENTPD1, ERG, PLXNA2, PODXL, FCHO2, PLXND1, PREX1, FBXL7, PKP4, EPB41L4A, PLCL2, PREX2, EPAS1, A2M, FLT1, MT-ND1, MKL2, MGAT4A, INPP5D, INSR, MEF2C, MEF2A, MECOM, MCTP1, ITGA6, ITGA9, MCF2L, IVNS1ABP, MTSS1, MARCH3, MAGI1, KALRN, LRMDA, KCNMA1, KCNN3, KCNQ1, KDR, KIAA1217, ZNF385D, LAMA4, LAMB1, LIMCH1, LDB2, JAG2, HSPG2, HOXD3, HMCN1, FLT4, FMNL3, FOLH1, PECAM1, PDGFD, FRMD4B, PDE10A, PCDH17, FYN, GAB1, GALNT18, PCAT19, PARVB, PALMD, NRP1, NR5A2, GNA14, NOX4, NOVA2, GRAMD1B, GRAPL, GRB10, NOTCH4, NOTCH1, NOSTRIN, NLK, NFIB, MYRIP, HECW2, PHACTR1, PRSS23, RASGRF2, LDLRAD3, ENG, CACHD1, SPARCL1, CADPS2, CALCRL, CARD8, CBLB, CC2D2B, ADAMTSL2, SNTB1, SNRK, ADARB1, SMAD1, CD93, ADAMTS9, CDH3, CDH5, SLCO2A1, CDK17, CEP112, UTRN, VEGFC, VWF, SHROOM4, CD34, AC074286.1, SPRY1, ST6GAL1, ARAP3, APP, TMCC3, ARHGAP29, ANO2, ARHGAP31, THSD7A, ANGPT2, ARHGEF15, TGFBR2, ARL15, SPTBN1, AL365273.2, TBCD, ATP11A, AFAP1L1, SWAP70, ADGRL4, ADGRL2, ADGRF5, ST8SIA4, BMPR2, ADCY4, ST6GALNAC3, TCF4, SHANK3, TMEM255B, SH2D3C, RASIP1, RASGRP3, DLEU2, DNM3, RASAL2, DOCK4, ABCB1, DOCK6, DOCK9, RAPGEF5, RAPGEF4, RAPGEF1, RAMP3, DYSF, PTPRM, PTPRG, PTPRB, PTPN14, EFNB2, EGFL7, ZNF366, EHD4, ELK3, ELMO1, EMCN, DGKH, RCAN2, DLL4, RIN2, RP1, ROBO4, CPNE5, RIN3, CSGALNACT1, ABCG1, RHO1, WWTR1, RGS3, CXorf36, SEC14L1, RGL1, AC010737.1, CYYR1, RGCC, AC002070.1, RFX3, SASH1, RDX.

### Gene Set 32

VDR, LINC01429, LRP1, TNFAIP6, LGALS1, MEG3, TMSB10, WISP1, LOXL1, TMEM45A, ZFHX4, UBE2M, TPI1, TPM1, TPM4, TRIT1, TUBA1A, XYLT1, LRIG1, IMO7, TUBA1B, UBC, TOX3, UGDH, PRRX1, MMP11, SLC6A6, PCDH7, SLC30A10, SLC24A3, SLC24A2, PDE4DIP, SIPA1L1, PDLIM7, SH3PXD2B, SLIT2, SGCD, PFN1, SALL4, RPL18, ROR2, RARRFS2, PLPP4, POSTN, PTMS, PTK7, SERPINE1, P4HA1, P3H1, SOX4, MMP14, TIMP3, M72A, TIMP1, MRA5, THBS2, MYH10, TENM4, MYL6, MYL9, TENM3, TAGLN2, TAGLN, SYNDIG1, SULF1, STEAP2, NAV3, ST6GALNAC5, SPP1, SPOCK1, SPARC, TMEM176A, SPHK1, LINC02257, FAP, IGHG1, BNC2, BMP1, GFPT2, BGN, IGHG3, IGHGP, IGKC, GBP1, IGLC2, DCBLD2, IGLC3, COL11A1, ATF3, ARL4C, INO80D, FTL, IGFBP5, IGFBP2, BX322234.1, C11orf96, GPC6, GREM1, GRIK2, CFL1, GSTP1, COL1A2, CDK14, CCDC80, F7T11, GLI2, CALU, HECW1, CTHRC1, C1QB, HMGA2, HMGB2, C1QA, HS3ST3A1, COL6A2, DPYSL3, DDR2, KCND2, FMN1, JUND, JUN, EVC, KIAA1755, ALPK2, AMIGO2, ACTB, FLNA, ANXA1, FN1, ENO2, ARFGAP1, ENC1, ADAM1S2, FKBP10, EFFAMP2, LAMB2, ADAMTS6, ITGBL1, KIAA0930, APOE, FOSB.

### Gene Set 33

COL5A3, CPM, CRTC3, SEP79, SEMA5A, SEPT11, FAM13C, PLXDC1, COL18A1, FAM182B, SH3KBP1, SH3RF1, PLSCR4, FAM20A, COL14A1, SDC2, PPM1L, EBF1, RGS5, PTPR1, DOK6, DOCK1, DOCK10, EDNRA, DLC1, EGFL4AM, RERG, ENOX1, PRKG1, RFTN1, ENPEP, EPB41L1, EPHA3, EPS8, DAAM2, ECE1, PPP1R12B, PPP1R12A, CYTOR, CYTH3, CSPG4, COBLL1, LARGE1, CNTN4, ARHGAP26, TLE1, ARHGAP24, ARHGAP17, TMEM165, ARHGAP15, ARHGAP10, TNC, ANO1, TNS3, AL603840.1, AHR, TRAK1, THY1, AGAP1, TRPC6, AFAP1L2, ADCY3, UACA, UBA2, ADAP2, UBE2E2, UST, ADAM12, WDFY2, ABCC9, ZEB2, ZFHX3, TRPC4, ARHGAP32, ARHGAP42, ARHGEF17, CNNM2, CLMN, SLC35F1, SLC6A1, CERS6, SLC8A1, SLCO3A1, CDH6, SLIT3, SMC4, CCDC3, SMURF1, CCDC102B, PLEKHA2, CALD1, CACNB2, CACNA1H, SPECC1, CACNA1C, SPRY4-AS1, STK39, SYTL2, TACC1, ATP2B4, ATP10A, TBX2, TEX41, TFP1, ARHGEF7, SLC12A2, CARMN, DPY19L2, NCK2, MAP1B, LZTS1, MOCS1, PAWR, NOTCH3, GALNT2, GRK3, GJC1, LURAP1L, GRM8, NHSL2, NHSL1, NRXN3, PDE1A, NFASC, PDE3A, GRK5, PALM2-AKAP2, MAP2, PAK1, MEG8, INPP4B, ITGA1, OAR, MIR4435-2HG, MCU, ITGA4, MCAM, ITPR1, MARK1, JAG1, MKL1, NR2F2-AS1, MAP4, MMP16, NR2F2, MAP3K20, NEURL1B, PDE5A, GTDC1, PDGFA, HEYL, HIP1, FILIP1, GUCY1B1, FILIP1L, LHFPL6, LAMC3, MYO1B, PEAK1, LINC01060, GUCY1A2, LINC01091, NBEAL1, FOXP1, KLHL29, KCNK6, MRVI1, GUCY1A1, FRMD4A, KIRREL1, FRMD3, KLF12, FAT1, PDGFRB, PLCE1, PLCL1, LIN7A.

### Gene Set 34

TBL1X, HOMER1, MICAL2, ATP2B1, MITF, HSD17B2, MGAT5, TEAD1, TBX3, ATP9A, IER3, MRC2, STXBP5, BMP5, SUGCT, MSC-AS1, BACH1, AXL, AUTS2, BTBD9, ATRNL1, BICC1, IGF1R, ST5, SSBP2, SRPX2, SPSB1, ARMC9, MAPKAPK2, INHBA, T7C3, AEBP1, KAZN, LRFN5, LPAR1, KIF26B, UBR4, UNC5C, ADAMTS12, VCAN, VEGFA, LSAMP, VMP1, LAMA2, WDR27, AC087564.1, AC016831.7, WNT5A, WNT5B, ABCC4, ABCA6, YAP1, ABCA10, ABCA1, ACSL4, IL1R1, TSHZ2, LTBP1, MEIS1, IGFB1, ARID5B, ARHGAP6, IRS1, ITGA11, ARHGAP28, MCTP2, TLE4, HIPK2, MAPK10, TRPS1, MAP3K5, TOP1, ANK3, MANBA, MAGI2, AL139383.1, AHI1, TPST1, TRABD2B, LUM, TRPA1, LTBP2, ANTXR1, SPON2, FARP1, SPIDR, ECHDC2, FRMD6, PDE4D, DST, LDLRAD4, DOCK5, FRY, PCNX1, FTX, DIP2C, RASSF8, EFCC1, DENND2A, DCN, GAS7, PARP14, DANT2, PARD3, PAPPA, PAMR1, PALLD, RHOBTB3, CSGALNACT2, CRISPLD2, RBMS3-AS3, PTPN13, PDGFC, EGFR, PLEKHA5, FAM20C, PLEKHH2, FENDR, FAM19A2, FGFR1, PLAU, PLAT, PLXDC2, FAM155A, FHL2, F3, PID1, PHLDA1, PRDM1, FOXO3, EPB41L2, PRICKLE2, PDZRN3, PDPN, PRR16, PDLIM3, PSD3, PTEN, EMD1, RIPOR3, RND3, PAG1, RNF152, SH3RF3, SHANK2, COL12A1, NR2F1-AS1, NR2F1, SLC14A1, NPAS2, GOLGA4, CLMP, CIP1, GTF2IRD1, CDH11, CD44, NEGR1, CCDC186, CCDC146, SNX9, SOBP, SON, GULP1, NAMPT, SOX5, SOX6, NAALADL2, C9orf3, GLIS3, C1S, COL6A1, GLIS1, CPED1, ROBO2, COL8A1, COL7A, RORA, COL6A3, GK, COL6A1, RUNX1, RUNX2, COL5A2, COL5A1, SAMD4A, COL4A5, SBF2, COL3A1, GLI3, SDK1, COL27A1, OSBPL10, OBSCN, NTM, SETBP1, COL1A1, SGIP1, NRG1, ZSWIM6.

1. A method for detecting a phenotype of a cancer or a gene expression pattern in the cancer in a subject, comprising:
(i) detecting the presence of a cadherin 12 (CDH12)-high phenotype in a cancer sample obtained from the subject;
(ii) detecting the presence of a cadherin 12 (CDH12)-low phenotype in the cancer sample;
(iii) detecting the presence of a keratin 6A (KRT6A)-high phenotype in the cancer sample;
(iv) detecting the presence of a cell-cycle-related (cycling)-high phenotype in the cancer sample;
(v) detecting the presence of a uroplakins (UPK)-high phenotype in the cancer sample;
(vi) detecting the presence of a keratin 13-and-keratin 17 (KRT)-high phenotype in the cancer sample;
(vii) detecting the presence of a gene expression pattern of latent time 0 in the cancer sample;
(viii) detecting the presence of a gene expression pattern of latent time 1 in the cancer sample;
(ix) detecting the presence of a gene expression pattern of latent time 2 in the cancer sample;
(x) detecting the presence of a gene expression pattern of latent time 3 in the cancer sample; and/or
(xi) detecting the presence of a gene expression pattern of latent time 4 in the cancer sample;
wherein detecting the presence of the CDH12-high phenotype comprises detecting a gene expression pattern comprising:
(a) an increased gene expression in at least 20, at least 50, at least 100, at least 200, at least 300, at least 400, at least 500, at least 600, at least 700, all 765, or at least one of the genes listed in Gene Set 1; and/or
(b) a gene mutation in at least one, at least two, at least three, at least four, at least five, at least six, or all seven of EIF4G3, ALAS1, NINL, NSD1, DFNA5, PABPC3, and TXNDC11;
wherein detecting the CDH12-low phenotype comprises detecting a gene expression pattern comprising:
(c) an increased gene expression in at least 20, at least 50, at least 100, all 124, or at least one of genes in Gene Set 2; and/or
(d) a gene mutation in at least one, at least three, at least five, at least ten, or all 12 of ERBB2, FGFR3, PAPPA2, ASAP1, OCA2, NDC80, AP3D1, BAP1, KIFAP3, NOC3L, PAX7, and TNRC18;
wherein detecting the KRT6A-high phenotype comprises detecting a gene expression pattern comprising an increased gene expression in at least 20, at least 25, at least 30, at least 40, or all 46 of the genes listed in Gene Set 3;
wherein detecting the cycling-high phenotype comprises detecting a gene expression pattern comprising an increased gene expression in at least 20, at least 50, at least 100, at least 200, or all 298 of the genes listed in Gene Set 4;
wherein detecting the UPK-high phenotype comprises detecting a gene expression pattern comprising an increased gene expression in at least 20, at least 50, at least 100, or all 187 of the genes listed in Gene Set 5;
wherein detecting the KRT-high phenotype comprises detecting a gene expression pattern comprising an increased gene expression in at least 20, at least 50, at least 100, at least 200, at least 300, at least 400, or all 419 of the genes listed in Gene Set 6;
wherein the gene expression pattern of latent time 0 comprises an increased gene expression in at least 20, at least 25, at least 30, at least 40, at least 50, at least 100 of, or all 178 of the genes listed in Gene Set 7;
wherein the gene expression pattern of latent time 1 comprises an increased gene expression in at least 20, at least 25, at least 30, at least 40, or all 47 of the genes listed in Gene Set 8;
wherein the gene expression pattern of latent time 2 comprises an increased gene expression in at least 20, at least 25, at least 30, at least 40, at least 50, at least 100, or all 160 of the genes listed in Gene Set 9;
wherein the gene expression pattern of latent time 3 comprises an increased gene expression in at least 20, at least 25, at least 30, at least 40, at least 50, at least 100, or all 160 of the genes listed in Gene Set 10; and
wherein the gene expression pattern of latent time 4 comprises an increased gene expression in at least 20, at least 25, at least 30, at least 40, at least 50, at least 100, or all 190 of the genes listed in Gene Set 11; and
wherein the increase or the decrease in gene expression levels are relative to a reference for each gene, and the increase in gene mutation is relative to a referenced mutation frequency for each gene.

2. The method of claim 1, detecting the presence of the CDH12-high phenotype in the cancer sample, wherein the detection detects:
an increased gene expression in at least the first 20, at least the first 25, at least the first 30, at least the first 40, at least the first 50, or at least the first 100 genes listed in the Gene Set 1; and/or
a gene mutation in one or more of at least EIF4G3, ALAS1, NINL, NSD1, DFNA5, PABPC3, and TXNDC11;
said Gene Set 1 listing the first 100 genes as follows: RBFOX1, CNTNAP2, CSMD1, DLG2, PTPRD, EYS, DPP10, PCDH15, CTNNA3, DMD, MT-CO1, LINC00486, CTNNA2, MT-CO3, FP700111.1, MT-CO2, TMEM132D, CDH12, GRID2, CSMD3, MT-ND4, CCDC26, CADM2, NRG1, MAGI2, CDH18, LRRC4C, ROBO2, CNTN5, AC007402.1, GPC5, LRP1B, ZFPM2, DCC, CALN1, GALNTL6, ANKS1B, KCNIP4, CNTN4, CDH13, MT-ND1, TENM2, CTNND2, TRPM3, NRXN1, C8orf37-AS1, MT-ATP6, CNTNAP5, RYR2, SORCS1, ZNF385D, AL589740.1, PRKG1, PTPRT, DLGAP1, CNBD1, PHACTR1, GPC6, AL138720.1, IL1RAPL1, OPCML, RALYL, PRKN, SOX5, ASIC2, AC034114.2, AC011287.1, USH2A, MT-ND3, CACNA1A, EPHA6, ADAMTSL1, MT-ND2, ERVMER61-1, AGBL1, MT-CYB, AC109466.1, MALRD1, DPP6, TBC1D19, NEGR1, NLGN1, DAB1, PCDH9, SUGCT, HPSE2, LINC02240, RGS7, HYDIN, GALNT17, PKN2-AS1, SNTG1, AFF3, LSAMP, DSCAM, MT-ND5, CPNE4, FRMD4A, ADGRL2, and SGCZ.

3. The method of claim 1, detecting the presence of the CDH12-low phenotype in the cancer sample, wherein the detection detects
a decreased gene expression in at least the first 20, at least the first 25, at least the first 30, at least the first 40, at least the first 50, or at least the first 100 genes listed in the Gene Set 2; and/or
a gene mutation in at least one or more of ERBB2, FGFR3, PAPPA2, ASAP1, OCA2, NDC80, AP3D1, BAP1, KIFAP3, NOC3L, PAX7, and TN7RC18;
said Gene Set 2 listing the first 100 genes as follows: TCIRG1, UNC93B1, HNRNPL, ORAOV1, PTP4A2, SLC2A1, SYNCRIP, NPIPB5, OFD1, SREBF1, EIF5, BCL6, AKAPI7A, CSAD, FOSB, TCIM, WEE1, CYP4F12, KDM3A, ANXA1, PPP1R10, HIP1R, CCNT2, BTBD3, IFI44, MAP3K8, SH3YL1, CLK1, ULK1, STARD3, SYTL1, CSNK1D, GRHL3, CYP3A5, MAOA, OSBPL2, EPHA2, TMEM259, ZFP36, AC106798.1, TRABD, UVSSA, MRPS6, PPP1CB, CEP95, UBE21, LTN1, TIAL1, RHOT2, C1orf159, FAM118A, NECTIN4, USP9Y, TMEM184A, CDK5RAP3, WASHC4, SFMA6A, APPL2, ZXDC, NECTIN1, YTHDC2, C3orf52, MTMR1, ZNF440, DAZAP1, TRIM38, DGKA, SRSF6, DMTF1, SUPT20H, COL7A1, CSNKIG2, SF1, MTX2, D2HGDH, GABPB1-AS1, ZNF326, PCF11, RAPGEFL1, ZDHHC3, MAP3K7, RBBP6, SHROOM1, KRT16, GOLGA3, PDCD6, RAB12, AC006978.2, CHMP4B, ENGASE, GBP2, PARD6B, WASL, RFC1, SIN3B, KIAA1522, HNRNPH3, LBR, SLC19A2, and MGAT1.

4. The method of claim 1, detecting the presence of the KRT6A-high phenotype in the cancer sample, wherein the detection detects an increased gene expression in at least the first 20, at least the first 25, at least the 30, or at least the first 40 genes listed in the Gene Set 3,
said Gene Set 3 listing the first 40 genes as follows: FP671120.1, FP236383.1, COL7A1, SFN, AC092683.1, AHNAK, CD44, SORCS2, PGGHG, PMEPA1, ANX41, S100A2, JAG1, MET, DSG3, OSMR, ANKRD36, KRT6A, AHNAK2, FLNA, XDH, AKR1C2, TNNI2, MTRNR2L8, CLIP4, SULF2, AC245060.5, PYGB, SSFA2, TYMP, DSC2, H1F0, ABCA7, KRT15, HMGA2, MYEOV, TFP1, CD109, S100A8, and KRT5.

5. The method of claim 1, detecting the presence of the cycling phenotype in the cancer sample, wherein the detection detects an increased gene expression in at least the first 20, at least the first 25, at least the first 30, at least the first 40, at least the first 50, or at least the 100 of the genes listed in the Gene Set 4,
said Gene Set 4 listing the first 100 genes as follows: AC104041.1, KCNMB2-AS1, SMC4, ARID1B, SCMH1, WWOX, AC009271.1, CEP192, CCDCl4, MIR4713HG, AC106798.1, LINC01748, SLCO3A1, TRA2B, GNGT1, WAC, LINC01572, FUS, BCL2, LINC02428, AC016205.1, NAP1L1, CENPF, EZH2, ASPM, PTBP2, FANCA, SSBP3, KAT6A, REV3L, HELLS, DANT2, ALCAM, SMAP2, TOP2A, ECT2, KCNB2, AKT3, FANC1, SCLT1, CTPS1, NFIB, TARBP1, C1QTNF3-AMACR, AC116049.2, LBR, CENPK, NEDD1, AC091057.6, L3MBTL4, TMPO, IGSF1, NFYC, RLF, SYT1, RAB12, ELOVL5, LINC01876, AP3M2, CD47, FOX13, RFC3, MKI67, MMS22L, NEO1, TRIT1, SMC6, Z94721.1, AL117329.1, GABPB1-AS1, CENPE, STK33, TCF4, KIF20B, DDX11, PAM, PRKD3, GEN1, RORA, AC092683.1, ANKRD6, NUF2, DPYSL3, ZEB1, CIP2A, IGSF9, POLQ, NCAPG2, CCDC18, SLF1, LYPLAL1, LINC00491, AC022031.2, CMC2, TTF2, NCAPG, C2Jorf58, ANKRD36, CIT, and AC073529.1.

6. The method of claim 1, detecting the presence of the UPK-high phenotype in the cancer sample, wherein the detection detects an increased gene expression in at least the first 20, at least the first 25, at least the first 30, at least the first 40, at least the first 50, or at least the 100 of the genes listed in the Gene Set 5,
said Gene Set 5 listing the first 100 genes as follows: CCSER1, PPARG, MECOM, ACER2, HPGD, DAPK1, CD96, NEAT1, AC087857.1, SNX31, RALGAPA2, BCAS1, PABPC1, LIMCH1, IKZF2, RBM47, AC009478.1, SCHLAP1, POF1B, CNGA1, SIDT1, THRB, SAMD12, PSCA, CMYA5, GATA3, CHKA, TNFRSF21, ABCD3, BICDL2, ELF3, MAML2, AC026167.1, RBPMS, ACOXL, SPTSSB, ICA1, PLPP1, ACOX1, MLPH, EPB41L1, GCLC, TBCID1, SLC20A1, ACSF2, EZR, ZNF254, NIPAL1, AC044810.3, GRAMD2B, SYTL2, SHROOM1, CD55, SPAG1, PPFIBP2, DAP, EHF, TMPRSS2, KCNJ15, ADGRF1, GPR39, C4orfl9, SLC44A3, ST3GAL5, SLC37A1, DOCK8, ZNF440, ALOX5, TBX2, SCCPDH, PKHD1, ENGASE, FU79, LIPH, TMEM45B, ACSL5, WWC1, SWAP70, RALBP1, VGLL3, SPTLC3, ABLIM3, RHE, SNCG, TMEM184A, GNA14, RARRES1, SLC19A2, ALAS1, NECTIN4, ZNF737, MAP3K8, PLIN5, SPINK1, NTN4, GPR160, BHMT, MAN1A1, GATA2-AS1, and CYP4F8.

7. The method of claim 1, detecting the presence of the KRT phenotype in the cancer sample, wherein the detection detects an increased gene expression in at least the first 20, at least the first 25, at least the first 30, at least the first 40, at least the first 50, or at least the 100 of the genes listed in Gene Set 6,
said Gene Set 6 listing the first 100 genes as follows: LINC00511, NEAT1, MAST4, RNF19A, VEGFA, VMP1, ZFAND3, CCNL1, TNFAIP2, KLF5, CSNK1A1, PTK2, ELF3, YWHAZ, THOC2, GRB7, RBM39, M7MR3, CMIP, SFMA4B, SMAD3, ATRX, NPEPPS, GRHL2, TOP2B, MECOM, VPS37B, CHD2, NCOA3, KTN1, ETS2, UTY, ETV6, PTPN13, PPP2R2A, SMURF1, GOLGA4, SON, TNFRSF21, KANSL1, NKTR, LINC00278, CD46, ERRFI1, RALGAPA2, ZFC3H1, SNX31, WSB1, TBX3, SLC14A1, ANKRD11, EZR, TCIRG1, TMEM51, TMPRSS4, KMT2E, NDRG1, SLC38A2, ZBTB7C, SLK, MID1, PPARG, ERBB2, ACTN4, SCHLAP1, SRSF11, KRT7, BRD4, ZMYM2, SRRM2, SERINC5, KDM6A, SFMA3C, PUM1, TMEM165, CCNL2, GATA3, LYPD6B, WDR45B, UBE3A, MARK3, ZSWIM6, TMEM117, UNC93B1, RNF149, EWSR1, CDH1, DYRK1A, USP3, HS6ST2, PTPRF, ADNP, TCF25, ZMYND8, KLF3, FOS, GOLGA8A, ATP8B1, ID1, and OGT.

8. The method of claim 1, wherein the method detects the presence of the CDH12-high phenotype and detecting an absence of one or more of the KRT6A-high phenotype, the cycling-high phenotype, the UPK-high phenotype, and the KRT-high phenotype, wherein detecting the absence of a phenotype is detecting the presence of an expression pattern other than that for the phenotype; or
wherein the method detects a higher percentage of the presence of the CDH12-high phenotype than that of the presence of each one of the KRT6A-high phenotype, the cycling-high phenotype, the UPK-high phenotype, and the KRT-high phenotype.

9. The method of claim 1, wherein the method detects an absence of CDH12-high phenotype and the presence of one or more of the KRT6A-high phenotype, the cycling-high phenotype, the UPK-high phenotype, and the KRT-high phenotype.

10. The method of claim 1, wherein the cancer comprises bladder cancer, muscle invasive bladder cancer (MIBC1, or urothelial carcinoma, and wherein at least 90%, 85%, 80%, or 75% of tumor cells in the cancer sample are epithelial cells or express a keratin.

11. (canceled)

12. The method of claim 1, wherein the cancer sample comprises a plurality of phenotypes, and the reference is two or more other phenotypes combined in the plurality or the reference is the plurality of the phenotypes combined.

13. (canceled)

14. The method of claim 1, wherein the reference is a non-cancerous sample from the subject or a sample from a subject without a cancer: or wherein the reference is another cancer sample obtained from the subject or from another subject.

15. (canceled)

16. A method for detecting a phenotype of a cancer or a gene expression pattern in the cancer in a subject, and treating, reducing the severity of and/or slowing the progression of the cancer in the subject, comprising:
detecting a phenotype of a cancer sample obtained from the subject or a gene expression pattern in the cancer sample according to claim 1, wherein the detection detects the presence of the CDH12-high phenotype and/or the presence of a gene expression pattern of latent time 0 or latent time 1 in the cancer sample; and
administering a therapeutically effective amount of an immune checkpoint inhibitor, a combination of the immune checkpoint inhibitor and a neoadjuvant chemotherapy, OR a transforming growth factor beta (TGFβ) inhibitor or an anti-angiogenic therapy, to the subject, thereby treating, reducing the severity of and/or slowing the progression of the cancer;
optionally wherein the subject's response to a chemotherapy in the absence of an immune checkpoint inhibitor therapy is ineffective.

17. (canceled)

18. A method for detecting a phenotype of a cancer or a gene expression pattern in the cancer in a subject, and treating, reducing the severity of and/or slowing the progression of the cancer, comprising:
detecting a phenotype of a cancer sample obtained from the subject or a gene expression pattern in the cancer sample according to claim 1, wherein the detection detects the presence of the CDH12-low phenotype, the absence of the CDH12-high phenotype, and/or the presence of a gene expression pattern of latent time 4 or latent time 3 in the cancer sample; and
administering a therapeutically effective amount of a chemotherapy to the subject and/or surgically removing the cancer from the subject, thereby treating, reducing the severity of and/or slowing the progression of the cancer.

19. The method of claim 18, followed by further detecting the presence of the CDH12-high phenotype in a remainder or relapsed cancer sample obtained from the subject, and administering a therapeutically effective amount of (1) an anti-PDL1 antibody or an anti-PD1 antibody, and/or (2) an anti-cytotoxic T-lymphocyte associated protein 4 (CTLA4) therapy, to the subject detected with the CDH12-high phenotype in the remainder or relapsed cancer sample; or
the method of claim 18 followed by further detecting the presence of the CDH12-low phenotype in the remainder or relapsed cancer sample from the subject, and administering a therapeutically effective amount of an anti-T cell immunoreceptor with Ig and ITIM domains (TIGIT) therapy or an anti-T-cell immunoglobulin and mucin domain 3 (TIM3) therapy to the subject detected with the CDH12-low phenotype in the remainder or relapsed cancer sample.

20. A method for treating, reducing the severity, of and/or slowing the progression of a cancer in a subject, comprising:
(i) administering a therapeutically effective amount of an immune checkpoint inhibitor to the subject, wherein the subject has been detected to have a CDH12-high expression pattern or a gene expression pattern of latent time 0 or latent time 1 in a cancer sample obtained from the subject according to the method of claim 1;
or
(ii) administering a therapeutically effective amount of a chemotherapy to the subject, wherein the subject has been detected to have a CDH12-low expression pattern or a gene expression pattern of latent time 4 or latent time 3 in the cancer sample from the subject according to the method of claim 1;
and wherein the increase in gene expression levels are relative to a reference for each gene.

21. The method of claim 20, wherein the immune checkpoint inhibitor comprises an anti-PD-L1 antibody or an anti-PD-1 antibody selected from atezolizumab, cemiplimab, nivolumab, pembrolizumab, avelumab, or duralumab, or a fragment thereof; and wherein the chemotherapy comprises cisplatin-based chemotherapy, optionally being one or more of (1) methotrexate, vinblastine, doxorubicin, and cisplatin (MVAC), (2) dose-dense, or accelerated, MVAC (ddMVAC), (3) gemcitabine and cisplatin (GC1, (4) paclitaxel, gemcitabine, and cisplatin (PGC1, and (5) cisplatin, methotrexate, and vinblastine (CMV).

22. (canceled)

23. A method for providing prognosis for a subject with a cancer, comprising:
detecting a CDH12-high phenotype or a CDH12-low phenotype in a cancer sample obtained from the subject, and/or detecting in the cancer sample a gene expression pattern of latent time 0, a gene expression pattern of latent time 1, a gene expression pattern of latent time 3, or a gene expression pattern of latent time 4, according to the method of claim 1;
and
providing a poorer survival prognosis, or a poorer responsiveness prognosis to a platinum-based chemotherapy optionally followed by a surgery, for the subject treated or to be treated with the platinum-based chemotherapy optionally followed by the surgery, relative to treatment with an immune checkpoint inhibitor or no treatment, based on a detected CDH12-high phenotype of the cancer sample from the subject,
providing a better survival prognosis, or a better responsiveness prognosis to the immune checkpoint inhibitor, for the subject treated or to be treated with the immune checkpoint inhibitor, relative to treatment with the platinum-based chemotherapy or no treatment, based on a detected CDH12-high phenotype and/or a detected gene expression pattern of latent time 0 or of latent time 1 in the cancer sample from the subject,
providing a better survival prognosis, or a better responsiveness prognosis to a neoadjuvant chemotherapy, for the subject treated or to be treated with the neoadjuvant chemotherapy, relative to no treatment for the subject, based on a detected CDH12-low phenotype of the cancer sample from the subject, or
providing a poorer survival prognosis, or a poorer responsiveness prognosis to the immune checkpoint inhibitor, for the subject treated or to be treated with the immune checkpoint inhibitor, relative to treatment with the platinum-based chemotherapy or no treatment, based on a detected gene expression pattern of latent time 4 or of latent time 3 in the cancer sample from the subject.

24. A method for treating, reducing the severity, and/or slowing the progression of a cancer in a subject, comprising performing a treatment based on a prognosis provided by a method of claim 23.

25. A method for classifying a cancer in a subject, comprising:
measuring a gene expression pattern in a cancer sample from the subject, and
classifying the cancer into a CDH12-high, a CDH12-low, a KRT6A-high, a cycling-high, a UPK-high, or a KRT-high phenotype, or classifying the cancer into a gene expression pattern of latent time 0, latent time 1, latent time 2, latent time 3, or latent time 4, based on the measured gene expression pattern in the cancer sample according to the method of claim 1,
wherein the gene expression pattern includes expression levels and/or mutation levels of a combination of genes in one or more of Gene Sets 1-6, or a combination of genes in one or more of Gene Sets 7-11.

26. The method of claim 25, wherein said measuring is performed by:
sequencing of mRNA, optionally unbiased sequencing, for measuring the expression levels;
sequencing of DNA, optionally unbiased sequencing, for measuring the mutation level;
or
contacting the cancer sample with one or more detection agents that specifically bind to each of the gene or a protein encoded by the gene; and
detecting the level of binding between the one or more detection agents and each of the gene or the protein encoded by the gene; wherein the one or more detection agents are oligonucleotide probes, nucleic acids, DNAs, RNAs, peptides, proteins, antibodies, aptamers, or small molecules, or a combination thereof.

27. A kit for detecting an expression pattern in a biological sample, classifying a cancer in a subject, and/or providing prognosis for the subject, comprising:
(i) one or more detection agents that specifically bind to each of a combination of at least the first 20, first 25, first 30, first 40, first 50, or first 100 genes of Gene Set 1 and/or proteins encoded thereby, wherein the first 100 genes of Gene Set 1 are RBFOX1, CNTNAP2, CSMD1, DLG2, PTPRD, EYS, DPP10, PCDH15, CTNNA3, DMD, MT-CO1, LINC00486, CTNNA2, MT-CO3, FP700111.1, MT-CO2, TMEM32D, CDH12, GRID2, CSMD3, MT-ND4, CCDC26, CADM2, NRG1, MAGI2, CDH18, LRRC4C, ROBO2, CNTN5, AC007402.1, GPC5, LRP1B, ZFPM2, DCC, CALN1, GALNTL6, ANKS1B, KCNIP4, CNTN4, CDH13, MT-ND1, TENM2, CTNND2, TRPM3, NRXN1, C8orf37-AS1, MT-ATP6, CNTNAP5, RYR2, SORCS1, ZNF385D, AL589740.1, PRKG1, PTPRT, DLGAP1, CNBD1, PHACTR1, GPC6, AL138720.1, ILIRAPL1, OPCML, RALYL, PRKN, SOX5, ASIC2, AC034114.2, AC011287.1, USH2A, MT-ND3, CACNA1A, EPHA6, ADAMTSL1, MT-ND2, ERVMER61-1, AGBL1, MT-CYB, AC109466.1, MALRD1, DPP6, TBC1D19, NEGR1, NLGN1, DAB1, PCDH9, SUGCT, HPSE2, LINC02240, RGS7, HYDIN, GALNT17, PKN2-AS1, SNTG1, AFF3, LSAMP, DSCAM, MT-ND5, CPNE4, FRMD4A, ADGRL2, and SGCZ;
one or more detection agents that specifically bind to each of a combination of at least the first 20, first 25, first 30, first 40, or all 46 genes of Gene Set 3 and/or proteins encoded thereby, wherein the 46 genes of Gene Set 3 are FP671120.1, FP236383.1, COL7A1, SFN, AC092683.1, AHNAK, CD44, SORCS2, PGGHG, PMEPA1, ANXA1, S100A2, JAG1, MET, DSG3, OSMR, ANKRD36, KRT6A, AHNAK2, FLNA, XDH, AKR1C2, TNNI2, MTRNR2L8, CLIP4, SULF2, AC245060.5, PYGB, SSFA2, TYMP, DSC2, H1F0, ABCA7, KRT15, HMGA2, MYEOV, TFP1, CD109, S100A8, KRT5, CDC25B, SAMD9L, FXYD5, SAMD9, CTSC, and CNTNAP3;
one or more detection agents that specifically bind to each of a combination of at least the first 20, first 25, first 30, first 40, first 50, or first 100 genes of Gene Set 4 and/or proteins encoded thereby, wherein the first 100 genes of Gene Set 4 are AC104041.1, KCNMB2-AS1, SMC4, ARID1B, SCMH1, WWOX, AC009271.1, CEP192, CCDC14, MIR4713HG, AC106798.1, LINC01748, SLCO3A1, TRA2B, GNGT1, WAC, LINC01572, FUS, BCL2, LINC02428, AC016205.1, NAP1L1, CENPF, EZH2, ASPM, PTBP2, FANCA, SSBP3, KAT6A, REV3L, HELLS, DANT2, ALCAM, SMAP2, TOP2A, ECT2, KCNB2, AKT3, FANCI, SCLT1, CTPS1, NFIB, TARBP1, C1QTNF3-AMACR, AC116049.2, LBR, CENPK, NEDD1, AC091057.6, L3MBTL4, TMPO, IGSF1, NFYC, RLF, SYT1, RAB12, ELOVL5, LINC01876, AP3M2, CD47, FOX13, RFC3, MKI67, MMS22L, NEO1, TRIT1, SMC6, Z94721.1, AL117329.1, GABPB1-AS1, CENPE, STK33, TCF4, KIF20B, DDX11, PAM, PRKD3, GEN1, RORA, AC092683.1, ANKRD6, NUF2, DPYSL3, ZEB1, CIP2A, IGSF9, POLQ, NCAPG2, CCDC18, SLF1, LYPLAL1, LINC00491, AC022031.2, CMC2, TTF2, NCAPG, C21orf58, ANKRD36, CIT, and AC073529.1;
one or more detection agents that specifically bind to each of a combination of at least the first 20, first 25, first 30, first 40, first 50, or first 100 genes of Gene Set 5 and/or proteins encoded thereby, wherein the first 100 genes of Gene Set 5 are CCSER1, PPARG, MECOM, ACER2, HPGD, DAPK1, CD96, NEAT1, AC087857.1, SNX31, RALGAPA2, BCAS1, PABPC1, LIMCH1, IKZF2, RBM47, AC009478.1, SCHLAP1, POF1B, CNGA1, SIDT1, THRB, SAMD12, PSCA, CMYA5, GATA3, CHKA, TNFRSF21, ABCD3, BICDL2, ELF3, MAML2, AC026167.1, RBPMS, ACOXL, SPTSSB, ICA1, PLPP1, ACOX1, MLPH, EPB41L1, GCLC, TBC1D1, SLC20A1, ACSF2, EZR, ZNF254, NIPAL1, AC044810.3, GRAMD2B, SYTL2, SHROOM1, CD55, SPAG1, PPFIBP2, DAP, EHF, TMPRSS2, KCN115, ADGRF1, GPR39, C4orf19, SLC44A3, ST3GAL5, SLC37A1, DOCK8, ZNF440, ALOX5, TBX2, SCCPDH, PKHD1, ENGASE, FUT9, LIPH, TMEM45B, ACSL5, WWC1, SWAP70, RALBP1, VGLL3, SPTLC3, ABLIM3, RHEX, SNCG, TMEM184A, GNA14, RARRES1, SLC19A2, ALAS1, NECTIN4, ZNF737, MAP3K8, PLIN5, SPINK1, NTN4, GPR160, BHMT, MAN1A1, GATA2-AS1, and CYP4F8;
one or more detection agents that specifically bind to each of a combination of at least the first 20, first 25, first 30, first 40, first 50, or first 100 genes of Gene Set 6 and/or proteins encoded thereby, wherein the first 100 genes of Gene Set 6 are LINC00511, NEAT1, MAST4, RNF19A, VEGFA, VMP1, ZFAND3, CCNL1, TNFAIP2, KLF5, CSNK1A1, PTK2, ELF3, YWHAZ, THOC2, GRB7, RBM39, MTMR3, CMIP, SFMA4B, SMAD3, ATRX, NPEPPS, GRHL2, TOP2B, MECOM, VPS37B, CHD2, NCOA3, KTN1, ETS2, UTY, ETV6, PTPN13, PPP2R2A, SMURF1, GOLGA4, SON, TNFRSF21, KANSL1, NKTR, LINC00278, CD46, ERRFI1, RALGAPA2, ZFC3H1, SNX31, WSB1, TBX3, SLC14A1, ANKRD11, EZR, TCIRG1, TMEM51, TMPRSS4, KMT2E, NDRG1, SLC38A2, ZBTB7C, SLK, MID1, PPARG, ERBB2, ACTN4, SCHLAP1, SRSF11, KRT7, BRD4, ZMYM2, SRRM2, SERINC5, KDM6A, SFMA3C, PUM1, TMEM165, CCNL2, GATA3, LYPD6B, WDR45B, UBE3A, MARK3, ZSWIM6, TMEM117, UNC93B1, RNF149, EWSR1, CDH1, DYRK1A, USP3, HS6ST2, PTPRF, ADNP, TCF25, ZMYND8, KLF3, FOS, GOLGA8A, ATP8B1, ID1, and OGT;
one or more detection agents that specifically bind to each of a combination of at least 20, at least 25, at least 30, at least 40, at least 50, at least 100 of, or all 178 of the genes listed in Gene Set 7 and/or proteins encoded thereby:
one or more detection agents that specifically bind to each of a combination of at least 20, at least 25, at least 30, at least 40, or all 47 of the genes listed in Gene Set 8 and/or proteins encoded thereby;
one or more detection agents that specifically bind to each of a combination of at least 20, at least 25, at least 30, at least 40, at least 50, at least 100, or all 160 of the genes listed in Gene Set 9 and/or proteins encoded thereby;
one or more detection agents that specifically bind to each of a combination of at least 20, at least 25, at least 30, at least 40, at least 50, at least 100, or all 160 of the genes listed in Gene Set 10 and/or proteins encoded thereby; and/or
one or more detection agents that specifically bind to each of a combination of at least 20, at least 25, at least 30, at least 40, at least 50, at least 100, or all 190 of the genes listed in Gene Set 11 and/or proteins encoded thereby; and
optionally (ii) instructions for using the one or more detection agents to detect the expression pattern in the biological sample, classify the cancer in the subject, and/or provide prognosis for the subject.

28. A system for treating, reducing the likelihood of having, reducing the severity of, and/or slowing the progression of a cancer in a subject, the system comprising:
(i) one or more detection agents in a kit according to claim 27 that specifically bind to each of a combination of at least the first 20, first 25, first 30, first 40, first 50, or first 100 genes of Gene Set 1 and/or proteins encoded thereby, wherein the first 100 genes of Gene Set 1 are RBFOX1, CNTNAP2, CSMD1, DLG2, PTPRD, EYS, DPP10, PCDH15, CTNNA3, DMD, MT-CO1, LINC00486, CTNNA2, MT-CO3, FP700111.1, MT-CO2, TMEM132D, CDH12, GRID2, CSMD3, MT-ND4, CCDC26, CADM2, NRG1, MAGI2, CDH18, LRRC4C, ROBO2, CNTN5, AC007402.1, GPC5, LRP1B, ZFPM2, DCC, CALN1, GALNTL6, ANKS1B, KCNIP4, CNTN4, CDH13, MT-ND1, TENM2, CTNND2, TRPM3, NRXN1, C8orf37-AS1, MT-ATP6, CNTNAP5, RYR2, SORCS1, ZNF385D, AL589740.1, PRKG1, PTPRT, DLGAP1, CNBD1, PHACTR1, GPC6, AL138720.1, ILIRAPL1, OPCML, RALYL, PRKN, SOX5, ASIC2, AC034114.2, AC011287.1, USH2A, MT-ND3, CACNA1A, EPHA6, ADAMISL1, MT-ND2, ERVMER61-1, AGBL1, MT-CYB, AC109466.1, MALRD1, DPP6, TBC1D19, NEGR1, NLGN1, DAB1, PCDH9, SUGCT, HPSE2, LINC02240, RGS7, HYDIN, GALNT17, PKN2-AS1, SNTG1, AFF3, LSAMP, DSCAM, MT-ND5, CPNE4, FRMD4A, ADGRL2, and SGCZ;
one or more detection agents in a kit according to claim 27 that specifically bind to each of a combination of at least 20, at least 25, at least 30, at least 40, at least 50, at least 100 of, or all 178 of the genes listed in Gene Set 7 and/or proteins encoded thereby; and/or
one or more detection agents in a kit according to claim 27 that specifically bind to each of a combination of at least 20, at least 25, at least 30, at least 40, or all 47 of the genes listed in Gene Set 8 and/or proteins encoded thereby; and
(ii) a quantity of a therapeutic comprising an immune checkpoint inhibitor;
and optionally (iii) instructions for using the one or more detection agents and the therapeutic to treat, reduce the likelihood of having, reduce the severity of, and/or slow the progression of the cancer in the subject.

29. A system for treating a subject having a cancer with a CDH12-high expression pattern, the system comprising:
(i) a quantity of a therapeutic comprising an immune checkpoint inhibitor, a TGFβ inhibitor, an anti-angiogenic therapy, or a combination thereof; and
(ii) one or more detection agents in a kit according to claim 27 that specifically bind to each of a combination of at least the first 20, first 25, first 30, first 40, first 50, or first 100 genes of Gene Set 1 and/or proteins encoded thereby, wherein the first 100 genes of Gene Set 1 are RBFOX1, CNTNAP2, CSMD1, DLG2, PTPRD, EYS, DPP10, PCDH15, CTNNA3, DMD, MT-CO1, LINC00486, CTNNA2, MT-CO3, FP700111.1, MT-CO2, TMEM132D, CDH12, GRID2, CSMD3, MT-ND4, CCDC26, CADM2, NRG1, MAGI2, CDH18, LRRC4C, ROBO2, CNTN5, AC007402.1, GPC5, LRP1B, ZFPM2, DCC, CALN1, GALNTL6, ANKS1B, KCNIP4, CNTN4, CDH13, MT-ND1, TENM2, CTNND2, TRPM3, NRXN1, C8orf37-AS1, MT-ATP6, CNTNAP5, RYR2, SORCS1, ZNF385D, AL589740.1, PRKG1, PTPRT, DLGAP1, CNBD1, PHACTR1, GPC6, AL138720.1, ILIRAPL1, OPCML, RALYL, PRKN, SOX5, ASIC2, AC034114.2, AC011287.1, USH2A, MT-ND3, CACNA1A, EPHA6, ADAMISL1, MT-ND2, ERVMER61-1, AGBL1, MT-CYB, AC109466.1, MALRD1, DPP6, TBC1D19, NEGR1, NLGN1, DAB1, PCDH9, SUGCT, HPSE2, LINC02240, RGS7, HYDIN, GALNT17, PKN2-AS1, SNTG1, AFF3, LSAMP, DSCAM, MT-ND5, CPNE4, FRMD4A, ADGRL2, and SGCZ; and
optionally (iii) instructions for using the therapeutic and the one or more detection agents to treat the subject having the cancer with the CDH12-high expression pattern;
wherein the CDH12-high expression pattern comprises an increased gene expression in the first 20, first 25, first 30, first 40, first 50, first 100, or at least one gene of Gene Set 1, relative to a reference level for each gene.

30. A gene selection method, comprising:
detecting expression levels for a combination of genes in each of a plurality of biological samples, wherein the combination of genes comprises those listed in two or more of Gene Sets 2-6, and wherein the plurality of biological samples are obtained from patients receiving a cancer therapy; and
identifying genes from the combination based on their detected expression levels or relative expression levels via a machine learning algorithm to correlate with each patient's response to the cancer therapy,
thereby selecting a set of genes associated with responsiveness to the cancer therapy;
optionally the gene selection method being for use in classifying a cancer patient and/or providing prognosis of responsiveness to the cancer therapy, wherein the cancer therapy comprises an immunotherapy and/or a chemotherapy.

31. The method of claim 30, wherein the machine learning algorithm comprises a Naïve Baees Classifier, a K-means Clustering, a Support Vector Machine, a Linear Regression, a Logistic Regression, an Artificial Neural Network, a Decision Trees, a Random Forrests, or a Nearest Neighbours algorithm.

32. (canceled)
